(19) United States
(12) Patent Application Publication
(10) Pub. No.: US 2026/0337246 A1
(43) Pub. Date: Oct. 29, 2026

(54) METHODS FOR TREATING OBESITY

(71) Applicant: Zealand Pharma A/S, Søborg (DK)

(72) Inventor: Jolanta SKARBALIENE, Søborg (DK)

(21) Appl. No.: 19/116,476

(22) PCT Filed: Sep. 28, 2023
(86) PCT No.: PCT/EP2023/076908

§ 371 Date: Mar. 28, 2025

Related U.S. Application Data
(60) Provisional application No. 63/410,847, filed on Sep. 28, 2022.

(51) Int. Cl. C07K 14/605; A61K 38/00; A61P 3/04

(52) U.S. Cl. CPC C07K 14/605; A61P 3/04; A61K 38/00

(57) ABSTRACT

The present invention features methods for treating obesity and promoting weight loss by administering to a subject a combination of a GLP-1 agonist and a GlP agonist.

### FIELD OF THE INVENTION

The invention features combination therapies for promoting weight loss.

### BACKGROUND OF THE INVENTION

Obesity is a chronic disease associated with many complications. Weight loss of 5-15% can improve many obesity-related complications. Despite the benefits of weight reduction, there are many challenges in losing weight and maintaining long-term weight loss. Achieving and maintaining weight loss is challenging for many individuals. There is therefore a need for some patients to take medications to help them lose weight and prevent weight regain.

GLP-1RA (GLP-1 receptor agonist) therapy was initially developed to treat type 2 diabetes. For example, owing to their effectiveness in reducing body weight, once-daily subcutaneous administration of liraglutide 3.0 mg and once weekly subcutaneous administration of semaglutide 2.4 mg have both been improved for weight management in people with obesity.

New approaches are needed for patients who fail to respond to GLP-1RA monotherapy, or who become refractory to GLP-1RA monotherapy during treatment, plateau in their weight loss, and/or are or become GLP-1 treatment resistant, while undergoing GLP-1RA treatment, or who wish to maintain weight loss following GLP-1RA monotherapy.

### SUMMARY OF THE INVENTION

The present invention features methods for treating obesity and promoting weight loss by administering to a subject a combination of a GLP-1 agonist and a GlP agonist. Applicants have observed that it is possible to continue weight loss in subjects undergoing GLP-1RA monotherapy reaching a weight loss plateau, and/or the subjects are or become GLP-1 treatment resistant, by adding a GlP agonist to their treatment regimen.

In a first aspect, the invention features a method of increasing weight loss in a subject in need thereof, the method including: (i) providing a subject undergoing treatment with a GLP-1 agonist for a period of time during which weight loss in the subject plateaus and/or the subject is or becomes GLP-1 treatment resistant, and (ii) following step (i), administering to the subject (a) the GLP-1 agonist and (b) a GlP agonist, each in an amount that together is sufficient to increase weight loss in the subject.

In a related aspect, the invention features a method of increasing weight loss in a subject in need thereof, the method including: (i) providing a subject who fails to respond to treatment with a GLP-1 agonist, and (ii) administering to the subject (a) the GLP-1 agonist and (b) a GlP agonist, each in an amount that together is sufficient to increase weight loss in the subject.

In a related aspect, the invention features a method of increasing weight loss in a subject in need thereof, the method including: (i) providing a subject undergoing treatment with a GLP-1 agonist for a period of at least 1 month (e.g., at least one month, two months, three months, four months, five months or six months), and (ii) following step (i), administering to the subject (a) the GLP-1 agonist and (b) a GlP agonist, each in an amount that together is sufficient to increase weight loss in the subject.

In a related aspect, the invention features a method of maintaining weight loss in a subject in need thereof, the method including: (i) providing a subject undergoing treatment with a GLP-1 agonist, wherein the subject has experience weight loss, and (ii) following step (i), administering to the subject (a) the GLP-1 agonist and (b) a GlP agonist, each in an amount that together is sufficient to maintain the weight loss in the subject. In particular embodiments, the subject's weight loss is maintained for a period of at least 1 month (e.g., at least one month, two months, three months, four months, five months or six months).

In an embodiment of any of the methods of the invention, the subject has a BMI of >30 kg/m2, >35 kg/m2, >40 kg/m2, >45 kg/m2, or >50 kg/m2.

In another embodiment of any of the methods of the invention, the GLP-1 agonist is selected from exenatide, liraglutide, semaglutide, dulaglutide, lixisenatide, albiglutide, and taspoglutide.

In another embodiment of any of the methods of the invention, the GlP agonist is any GlP agonist described herein. In particular embodiments, the GlP agonist is H-Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

In particular embodiments of any of the methods of the invention, during part (i) of the method the subject is not receiving any other weight loss drug other than the GLP-1 agonist.

### A. DEFINITIONS

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well known and commonly used in the art.

Unless specified otherwise, the following definitions are provided for specific terms, which are used in the above written description.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

The term “agonist” as employed in the context of the invention refers to a substance (ligand) that activates signaling by the receptor type in question.

Throughout the description and claims the conventional one-letter and three-letter codes for natural (or “proteinogenic”) amino acids are used, as well as generally accepted three letter codes for other (non-natural or “non-proteinogenic”) α-amino acids, such as Aib (α-aminoisobutyric acid), Orn (ornithine) and D-Ala (D-alanine). All amino acid residues in peptides of the invention are preferably of the L-configuration except where explicitly stated. Among sequences disclosed herein are sequences incorporating an “H-” moiety at the amino terminus (N-terminus) of the sequence, and either an “—OH” moiety or an “—NH₂” moiety at the carboxy terminus (C-terminus) of the sequence. In such cases, and unless otherwise indicated, an “H—” or “Hy-” moiety at the N-terminus of the sequence in question indicates a hydrogen atom (i.e. R¹=H), corresponding to the presence of a free primary or secondary amino group at the N-terminus, while an “—OH” or an “—NH₂” moiety at the C-terminus of the sequence (i.e. R²=OH or NH₂) indicates a carboxy (COOH) group or an amido (CONH₂) group at the C-terminus, respectively.

The term “pharmaceutically acceptable salt” refers to a salt of the compound. Salts include pharmaceutically acceptable salts, such as, e.g., acid addition salts and basic salts. Examples of an acid addition salt includes hydrochloride salts. Examples of basic salts include salts where the cation is selected from, e.g., an alkali metal. Other examples of pharmaceutically acceptable salts are described in “Remington's Pharmaceutical Sciences”, 17th edition. Ed. Alfonso R. Gennaro (Ed.), Mark Publishing Company, Easton, PA, U.S.A., 1985 and more recent editions, and in the Encyclopaedia of Pharmaceutical Technology.

“Treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” may also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures in certain embodiments. Those in need of treatment include those already with the disorder as well as those in which the disorder is to be prevented. By treatment is meant inhibiting or reducing an increase in pathology or symptoms (e.g. weight gain) when compared to the absence of treatment, and is not necessarily meant to imply complete cessation of the relevant condition.

As used herein, the term “plateau” refers to patients undergoing GLP-1 monotherapy for a period of time during which the subject's weight is unchanged, or there is weight gain in the subject. A subject experiencing a plateau can be identified when their weight loss is less than 1% of their body weight over a period of at least two weeks, three weeks, one month, or two months.

As used herein, the term “GLP-1 treatment resistant” refers to patients undergoing GLP-1 monotherapy for a period of time during which the subject's weight is unchanged, or there is weight gain in the subject. For some aspects of the invention, a subject experiencing such a resistance can be identified as any one or more of the following weight characteristics of the subject over a period of any of: at least about one week, or at least about two weeks, or at least about three weeks, or at least about one month, or at least about two months, about one week, or about two weeks, or about three weeks, or about one month, or about two months: when their weight loss is less than about 3%, or when their weight loss is less than about 2%, or when their weight loss is less than about 1% of their body weight, or when there is a substantially constant body weight, or when there is an increase in body weight of about at least 1%. In some instances, patients that experience a plateau may be a sub-group of patients who are GLP-1 treatment resistant.

As used herein, the term “fails to respond to treatment with a GLP-1 agonist” refers to patients undergoing GLP-1 monotherapy for a period of at least 1 month or at least 2 months without experiencing a loss of at least 1% of their body weight.

In either the treatment of an overweight or obese subject, the effect of treatment according to the methods of the present invention may be superior to that obtained GLP-1 agonist monotherapy or GlP agonist monotherapy in comparable subjects.

### B. BRIEF DESCRIPTION OF THE DRAWINGS

FIG. 1 is a graph showing that the combination therapy of semaglutide+compound 41, achieved a significant, sustained and greater body weight reduction in obese mice (DIO mice) compared to vehicle- and each monotherapy group (−24.5%±2.4 combination therapy from day 14; −24.1%±1.3 combination therapy from day 0; −0.8%±0.9 compound 41 from day 0; 2.0%±0.8 compound 41 from day 14 days; −13.7%±2.3 semaglutide; 3.1%±0.8, vehicle; relative to initial body weights). The effects on body weight loss in combination groups of semaglutide±compound 41 were similar at the end of the study irrespective of the pretreatment with semaglutide. The results were obtained as described in Example 1.

### DETAILED DESCRIPTION

The present invention features methods for treating obesity and promoting weight loss by administering to a subject a combination of an GLP-1 agonist and a GlP agonist. The methods of the invention can be useful for promoting weight loss in (i) subjects who are refractory to GLP-1 monotherapy, and (ii) subjects experiencing a plateau in weight loss in response to ongoing GLP-1 monotherapy and/or subjects that are or become GLP-1 treatment resistant.

### C. GLP-1 RECEPTOR AGONISTS

The combination therapies of the present invention may be particular effective in improving glycaemic control and reducing body weight. Glucagon-like peptide-1 receptor agonists also known as GLP-1 receptor agonists or incretin mimetics are agonists of the GLP-1 receptor. One of their advantages over older insulin secretagogues, such as sulfonylureas or meglitinides, is that they have a lower risk of causing hypoglycemia. Examples of GLP-1 agonists that can be used in the methods of the invention include but are not limited to exenatide (BYETTA®/BYDUREON®), liraglutide (VICTOZA®), semaglutide, lixisenatide (LYXUMIA®), dulaglutide (TRULICITY®), albiglutide (TANZENUM®) and Taspoglutide. The GLP-1 agonist used in the combination therapy of the invention can be a peptide having the sequence of any one of exenatide, liraglutide, semaglutide, lixisenatide, dulaglutide, albiglutide, or taspoglutide, or a pharmaceutically acceptable salt thereof.

The GLP-1 agonist may be any GLP-1 agonist described herein or a pharmaceutically acceptable salt thereof.

Exenatide is a synthetic version of exendin-4, a peptide found in the venom of the Gila monster, described in FIG. 2 of WO 99/07404 and has the formula:

(SEQ ID NO: 89)
Hy-HGEGTFTSDLSKQMEEEAVRLFIEWLKNGGPSSGAPPP-NH₂.

Liraglutide is a derivative of GLP-1 described in Example 37 of U.S. Pat. No. 6,268,343 and has the formula:

(SEQ ID NO: 90)
Hy-HAEGTFTSDVSSYLEGQAAKEFIAWLVRGRG-OH,

wherein the side-chain amino group of the lysine at position 20 is linked to the lipohilic moiety hexadecanoyl (i.e. palmitoyl) via a γ-Glu linker.

Semaglutide is a derivative of GLP-1 described in Example 4 of WO 2006/097537 and has the formula:

(SEQ ID NO: 91)
Hy-HXEGTFTSDVSSYLEGQAAKEFIAWLVRGRG-OH,

wherein X at position 2 is 2-aminoisobutyric acid (Aib) and the side-chain amino group of the lysine at position 20 is linked to the lipohilic moiety [2-(2-[2-(2-[2-(2-[4-(17-Carboxyheptadecanoylamino)-4 (S)-carboxybutyrylamino]ethoxy) ethoxy]acetylamino) ethoxy]ethoxy) acetyl].

Dulaglutide is a fusion protein of a GLP-1 variant and an IgG4 Fc region described in WO 2005/000892 (compound named “Gly⁸-Glu²²-Gly³⁶-GLP-1(7-37)-L-IgG4(S228P,F234A,L235A)”) and has the sequence:

(SEQ ID NO: 92)
HGEGTFTSDVSSYLEEQAAKEFIAWLVKGGGGGGGSGGGGSGGGGS
AESKYGPPCPPCPAPEAAGGPSVFLFPPKPKDTLMISRTPEVTCV
VVDVSQEDPEVQFNWYVDGVEVHNAKTKPREEQFNSTYRVVSVLT
VLHQDWLNGKEYKCKVSNKGLPSSIEKTISKAKGQPREPQVYTLP
PSQEEMTKNQVSLTCLVKGFYPSDIAVEWESNGQPENNYKTTPPV
LDSDGSFFLYSRLTVDKSRWQEGNVFSCSVMHEALHNHYTQKSLS
LSLG.

Lixisenatide is a derivative of exendin-4, presented as Compound 2 in WO 01/04156 and has the formula:

(SEQ ID NO: 93)
Hy-HGEGTFTSDLSKQMEEEAVRLFIEWLKNGG
PSSGAPPSKKKKKK-NH₂.

Albiglutide is Construct 3438 in WO 2005/003296 and has the formula:

(SEQ ID NO: 94)
HGEGTFTSDVSSYLEGQAAKEFIAWLVKGRHGEGTFTSDVSSYLE
GQAAKEFIAWLVKGRDAHKSEVAHRFKDLGEENFKALVLIAFAQY
LQQCPFEDHVKLVNEVTEFAKTCVADESAENCDKSLHTLFGDKLC
TVATLRETYGEMADCCAKQEPERNECFLQHKDDNPNLPRLVRPEV
DVMCTAFHDNEETFLKKYLYEIARRHPYFYAPELLFFAKRYKAAF
TECCQAADKAACLLPKLDELRDEGKASSAKQRLKCASLQKFGERA
FKAWAVARLSQRFPKAEFAEVSKLVTDLTKVHTECCHGDLLECAD
DRADLAKYICENQDSISSKLKECCEKPLLEKSHCIAEVENDEMPA
DLPSLAADFVESKDVCKNYAEAKDVFLGMFLYEYARRHPDYSVVL
LLRLAKTYETTLEKCCAAADPHECYAKVFDEFKPLVEEPQNLIKQ
NCELFEQLGEYKFQNALLVRYTKKVPQVSTPTLVEVSRNLGKVGS
KCCKHPEAKRMPCAEDYLSVVLNQLCVLHEKTPVSDRVTKCCTES
LVNRRPCFSALEVDETYVPKEFNAETFTFHADICTLSEKERQIKK
QTALVELVKHKPKATKEQLKAVMDDFAAFVEKCCKADDKETCFAE
EGKKLVAASQAALGL.

Albiglutide is a fusion protein of two copies of a derivative of GLP-1 fused in series, fused to human serum albumin at their C-terminus.

Taspoglutide is a GLP-1 variant described in Example 1 of WO 2000/34331 and has the formula:

(SEQ ID NO: 95)
Hy-HXEGTFTSDVSSYLEGQAAKEFIAWLVKXR-NH₂,

wherein X at positions 2 and 29 is 2-aminoisobutyric acid (Aib).
a. Route of Administration of GLP-1 Agonist

The administration to a subject of the GLP-1 agonist may be by any mode of administration common or standard in the art, e.g. oral, intravenous, intramuscular, subcutaneous, sublingual, intranasal, intradermal, suppository routes or implanting. The GLP-1 agonist of step (i) and/or step (ii) may be administered by one or more of the following routes of administration: injection (including subcutaneous injection, intramuscular injection, intravenous injection, epidural injection and intraperitoneal injection), infusion pump (such as subcutaneous infusion pump), oral, sublingual, rectal, vaginal, parenteral, topical, dermal (such as by transdermal patch), nasal (such as by nasal spray) and pulmonary spray. In certain embodiments the GLP-1 agonist may be administered by subcutaneous injection.

b. Dose of GLP-1 Agonist

The GLP-1 agonist of step (i) and/or step (ii) may be administered at a dose of up to about 50 mg, such as up to about 40 mg, up to about 30 mg, up to about 20 mg or up to about 10 mg.

The GLP-1 agonist of step (i) and/or step (ii) may be administered at a dose of from of from about 0.25 mg to about 100 mg, such as from about 0.25 mg to about 90 mg, such as from about 0.25 mg to about 80 mg, such as from about 0.25 mg to about 70 mg, such as from about 0.25 mg to about 60 mg, about 0.25 mg to about 50 mg, such as from about 0.25 mg to about 40 mg, such as from about 0.25 mg to about 30 mg, from about 0.25 mg to about 20 mg, from about 0.25 mg to about 14 mg or from about 0.25 mg to about 10 mg. The GLP-1 agonist of step (i) and/or step (ii) may be administered at a dose of from about 1 mg to about 50 mg, such as from about 1 mg to about 30 mg, from about 1 mg to about 20 mg, from about 1 mg to about 14 mg or from about 1 mg to about 10 mg.

The GLP-1 agonist of step (i) and/or step (ii) may be administered at a dose of about 0.25 mg, about 0.5 mg, about 0.6 mg, about 1 mg, about 1.2 mg, about 1.5 mg, about 1.8 mg, about 2 mg, about 3 mg, about 4.5 mg, about 5 mg, about 7 mg, about 10 mg, about 14 mg, about 20 mg, about 30 mg or about 50 mg.

The GLP-1 agonist of step (i) and/or step (ii) may be administered at a dose of up to about 10 mg/kg body weight of the subject, such as up to about 5 mg/kg body weight or up to about 1 mg/kg body weight.

The GLP-1 agonist of step (i) and/or step (ii) may be administered at a dose of from about 0.01 to about 10 mg/kg body weight of the subject, such as from about 0.01 to about 5 mg/kg body weight, from about 0.01 to about 1 mg/kg body weight, from about 0.05 to about 10 mg/kg body weight, from about 0.05 to about 5 mg/kg body weight, from about 0.05 to about 1 mg/kg body weight or from about 0.05 to about 0.5 mg/kg body weight.

c. Timing of Administration of GLP-1 Agonist

The GLP-1 agonist of step (i) and/or step (ii) may be administered to the subject at least once, twice, or three times daily. The GLP-1 agonist of step (i) and/or step (ii) may be administered to the subject once every 2 days, every 3 days, every 4 days, every 5 days, every 6 days, every 7 days, every 8 days, every 9 days, every 10 days, every 11 days, every 12 days, every 13 days, every 14 days or more such as every 28 days or every month. For certain embodiments, the GLP-1 agonist of step (i) and/or step (ii) may be administered to the subject once a day (i.e. daily or once daily). For certain embodiments, the GLP-1 agonist of step (i) and step (ii) may be administered to the subject once a week (i.e weekly or once weekly).

Exenatide is preferably administered by subcutaneous injection twice daily, at least 6 hours apart and each time before a main meal. Initially, 5 mg are administered twice daily for at least 1 month, then the dose may be increased to 10 mg twice daily if necessary. Alternatively, exenatide may be administered by subcutaneous injection once weekly at a dose of 2 mg.

Liraglutide is preferably administered by subcutaneous injection once daily. Initially, 0.6 mg are administered once daily for at least 1 week, then the dose is increased to 1.2 mg once daily for at least 1 week, and then the dose may be increased to 1.8 mg once daily if necessary.

Semaglutide is preferably administered by subcutaneous injection once weekly. Semaglutide may be administered subcutaneously at a dose of up to about 7.2 mg. In a preferred embodiment, 0.25 mg are initially administered once weekly for at least 4 weeks, then the dose is increased to 0.5 mg once weekly for at least 4 weeks, then the dose is increased to 1 mg once weekly for at least 4 weeks, then the dose is increased to 1.7 mg once weekly for at least 4 weeks, and then the dose is increased to 2.4 mg once weekly; the dose is maintained at 2.4 mg for the course of the treatment.

Alternatively, semaglutide may be administered orally once daily on an empty stomach. Semaglutide may be administered orally once daily at a dose of up to about 50 mg, such as about 50 mg, about 40 mg, about 30 mg, about 20 mg or about 10 mg. In a preferred embodiment, 3 mg are initially administered once daily for at least 1 month, then the dose is increased to 7 mg once daily for at least 1 month, and then the dose may be increased to 14 mg once daily if necessary.

Dulaglutide is preferably administered by subcutaneous injection once weekly at a dose of 0.75 mg. Alternatively, if dulaglutide is administered in combination with another drug (such as a GlP agonist), dulaglutide is preferably administered initially at 1.5 mg once weekly for at least 4 weeks, then the dose is increased to 3 mg once weekly for at least 4 weeks, and then the dose may be increased to 4.5 mg once weekly if necessary.

Lixisenatide is preferably administered by subcutaneous injection once daily, taken within 1 hour before a meal. Initially, 10 mg are administered once daily for 2 weeks, then the dose is increased to 20 mg once daily.

Albiglutide is preferably administered by subcutaneous injection once weekly at a dose of 30 mg. The dose may be increased to 50 mg if the glycaemic response is inadequate.

Taspoglutide is preferably administered by subcutaneous injection once weekly.

d. GlP Receptor Agonists

The combination therapies of the invention include a GlP analogue (also referred to as a “GlP agonist” or a “GlP receptor agonist” herein).

The GlP agonist may be any GlP agonist described herein or a pharmaceutically acceptable salt thereof.

The combination therapies of the invention can include a GlP analogue represented by the general Formula I:

(I)
(SEQ ID NO: 42)
R¹-Tyr-X2-Glu-Gly-Thr-Phe-Ile-Ser-Asp-X10-
X11-X12-Glu-Leu-X15-X16-X17-X18-X19-X20-X21-
Phe-X23-X24-X25-Leu-X27-X28-X29-Y1-Y2-R²

wherein
R¹ is H—, Ac or pGlu pyroglutamic acid (pGlu; (S)-(−)-2-pyrrolidone-5-carboxylic acid),
C₁₋₄ alkyl, acetyl, formyl, benzoyl and trifluoroacetyl,
X2 is Aib, Ala, D-Ala, Gly, Ser, N-Me-Ser, Ac3c, Ac4c or Ac5c;
X10 is Tyr, Leu or Ser;
X11 is Ser or Leu;
X12 is Lys, ψ or Ile;
X15 is Asp or Glu;
X16 is Ser, Glu, Lys or ψ;
X17 is Ile, Lys, Gln, Arg or ψ;
X18 is His, Arg or Ala;
X19 is Gln, Lys, Ala or Glu;
X20 is Gln, Lys, Ala, His or Arg;
X21 is Ala, Leu, Asp or Glu;
X23 is Val or Ile;
X24 is Asn or Glu;
X25 is Tyr or Trp;
X27 is Leu, Glu, Ser, Lys or Val;
X28 is Ala, Ser or Arg;
X29 is Aib, Gly, Ala, Gln, Thr, Ser or Lys or is absent;
Y1 is Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), Gly-Lys-Lys-Asn-Asp-Trp-Lys-His-Asn-Ile-Thr-Gln (SEQ ID NO: 47) or absent;
Y2 is ψ or is absent;
R² is —NH₂ or —OH;
   wherein ψ is a residue independently selected from Lys, Arg, Orn and Cys and wherein the side chain of said residue is conjugated to a lipophilic substituent;
      and wherein the GlP analogue contains one and only one residue ψ;
      or a pharmaceutically acceptable salt thereof.

Thus, a GlP analogue according to Formula I may be any of the following:

(SEQ ID NO: 115)
R¹-Tyr-X2-Glu-Gly-Thr-Phe-Ile-Ser-Asp-X10-X11-X12-Glu-Leu-X15-X16-X17-X18-
X19-X20-X21-Phe-X23-X24-X25-Leu-X27-X28-X29-Lys-Gly-Y2-R²;
(SEQ ID NO: 97)
R¹-Tyr-X2-Glu-Gly-Thr-Phe-Ile-Ser-Asp-X10-X11-X12-Glu-Leu-X15-X16-X17-X18-
X19-X20-X21-Phe-X23-X24-X25-Leu-X27-X28-X29-Gly-Pro-Ser-Ser-Gly-Ala-Pro-
Pro-Pro-Ser-Y2-R²;
(SEQ ID NO: 98)
R¹-Tyr-X2-Glu-Gly-Thr-Phe-Ile-Ser-Asp-X10-X11-X12-Glu-Leu-X15-X16-X17-X18-
X19-X20-X21-Phe-X23-X24-X25-Leu-X27-X28-X29-Gly-Pro-Ser-Ser-Gly-Ala-Pro-
Pro-Ser-Y2-R²;
(SEQ ID NO: 99)
R¹-Tyr-X2-Glu-Gly-Thr-Phe-Ile-Ser-Asp-X10-X11-X12-Glu-Leu-X15-X16-X17-X18-
X19-X20-X21-Phe-X23-X24-X25-Leu-X27-X28-X29-Pro-Ser-Ser-Gly-Ala-Pro-Pro-
Pro-Ser-Y2-R²;
(SEQ ID NO: 100)
R¹-Tyr-X2-Glu-Gly-Thr-Phe-Ile-Ser-Asp-X10-X11-X12-Glu-Leu-X15-X16-X17-X18-
X19-X20-X21-Phe-X23-X24-X25-Leu-X27-X28-X29-Pro-Ser-Ser-Gly-Ala-Pro-Pro-
Ser-Y2-R²;
(SEQ ID NO: 101)
R¹-Tyr-X2-Glu-Gly-Thr-Phe-Ile-Ser-Asp-X10-X11-X12-Glu-Leu-X15-X16-X17-X18-
X19-X20-X21-Phe-X23-X24-X25-Leu-X27-X28-X29-Gly-Lys-Lys-Asn-Asp-Trp-Lys-
His-Asn-Ile-Thr-Gln-Y2-R²;
or
(SEQ ID NO: 102)
R¹-Tyr-X2-Glu-Gly-Thr-Phe-Ile-Ser-Asp-X10-X11-X12-Glu-Leu-X15-X16-X17-X18-
X19-X20-X21-Phe-X23-X24-X25-Leu-X27-X28-X29-Y2-R².

In one aspect, R¹ is H—, Ac or pGlu.

Combinations of residues which may be present at some of the variable positions of Formula I include:
Aib2, Asp15, Lys20;
Aib2, Asp15, Arg20;
Aib2, Asp15, Arg20, Ile23;
Aib2, Ile12, Asp15, Arg20, Ile23, Glu24;
Ile12, Asp15, Ile23;
Ile12, Asp15, Ile23, Glu24;
Ile12, Asp15, Ala21, Ile23;
Aib2, Ala21, Ile23, Glu24;
Aib2, Asp15, Ile23;
Aib2, Asp15, Arg20, Ile23, Gln29;
Aib2, Asp15, Arg20, Gly29;
Aib2, Asp15, Ile17, Arg20, Gly29;
Aib2, Asp15, Ile17, Lys20, Gly29;
DAla2, Asp15, Ile23;
DAla2, Asp15, Ile23, Ala28;
Aib2, Asp15, Ile17, Lys20, Ala28;
Asp15, Ile23, Glu24;
N-Me-Ser2, Asp15, Lys20;
N-Me-Ser2, Asp15, Arg20;
N-Me-Ser2, Asp15, Arg20, Ile23;
N-Me-Ser2, Ile12, Asp15, Arg20, Ile23, Glu24;
N-Me-Ser2, Ala21, Ile23, Glu24;
N-Me-Ser2, Asp15, Ile23;
N-Me-Ser2, Asp15, Arg20, Ile23, Gln29;
N-Me-Ser2, Asp15, Arg20, Gly29;
N-Me-Ser2, Asp15, Ile17, Arg20, Gly29;
N-Me-Ser2, Asp15, Ile17, Lys20, Gly29;
N-Me-Ser2, Asp15, Ile23;
N-Me-Ser2, Asp15, Ile23, Ala28;
Ac3c2, Asp15, Lys20;
Ac3c2, Asp15, Arg20;
Ac3c2, Asp15, Arg20, Ile23;
Ac3c2, Ile12, Asp15, Arg20, Ile23, Glu24;
Ac3c2, Ala21, Ile23, Glu24;
Ac3c2, Asp15, Ile23;
Ac3c2, Asp15, Arg20, Ile23, Gln29;
Ac3c2, Asp15, Arg20, Gly29;
Ac3c2, Asp15, Ile17, Arg20, Gly29;
Ac3c2, Asp15, Ile17, Lys20, Gly29;
Ac3c2, Asp15, Ile23;
Ac3c2, Asp15, Ile23, Ala28;
Ac4c2, Asp15, Lys20;
Ac4c2, Asp15, Arg20;
Ac4c2, Asp15, Arg20, Ile23;
Ac4c2, Ile12, Asp15, Arg20, Ile23, Glu24;
Ac4c2, Ala21, Ile23, Glu24;
Ac4c2, Asp15, Ile23;
Ac4c2, Asp15, Arg20, Ile23, Gln29;
Ac4c2, Asp15, Arg20, Gly29;
Ac4c2, Asp15, Ile17, Arg20, Gly29;
Ac4c2, Asp15, Ile17, Lys20, Gly29;
Ac4c2, Asp15, Ile23;
Ac4c2, Asp15, Ile23, Ala28;
Ac5c2, Asp15, Lys20;
Ac5c2, Asp15, Arg20;
Ac5c2, Asp15, Arg20, Ile23;
Ac5c2, Ile12, Asp15, Arg20, Ile23, Glu24;
Ac5c2, Ala21, Ile23, Glu24;
Ac5c2, Asp15, Ile23;
Ac5c2, Asp15, Arg20, Ile23, Gln29;
Ac5c2, Asp 15, Arg20, Gly29;
Ac5c2, Asp15, Ile17, Arg20, Gly29;
Ac5c2, Asp15, Ile17, Lys20, Gly29;
Ac5c2, Asp15, Ile23; or
Ac5c2, Asp15, Ile23, Ala28.

The combination therapies of the invention can include a GlP analogue represented by the general Formula II:

(II)
(SEQ ID NO: 48)
R¹-Tyr-X2-Glu-Gly-Thr-Phe-Ile-Ser-Asp-Tyr-
Ser-X12-Glu-Leu-X15-X16-X17-X18-X19-X20-X21-
Phe-X23-X24-X25-Leu-X27-X28-X29-Y1-Y2-R²

wherein
R¹ is H—, Ac or pGlu;
X2 is Aib, Ala, D-Ala or Gly;
X12 is Lys, ψ or Ile;
X15 is Asp or Glu;
X16 is Ser, Glu, Lys or ψ;
X17 is Ile, Lys, Gln, Arg or ψ;
X18 is His, Arg or Ala;
X19 is Gln or Ala;
X20 is Gln, Lys, Ala, His or Arg;
X21 is Ala, Asp or Glu;
X23 is Ile or Val;
X24 is Asn or Glu;
X25 is Tyr or Trp;
X27 is Leu, Glu, Ser, Lys or Val;
X28 is Ala, Ser or Arg;
X29 is Aib, Gly, Ala, Gln, Thr, Ser or Lys or is absent;
Y1 is Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), Gly-Lys-Lys-Asn-Asp-Trp-Lys-His-Asn-Ile-Thr-Gln (SEQ ID NO: 47) or absent;
Y2 is ψ or is absent;
R² is —NH₂ or —OH;
   wherein ψ is a Lys residue ψ herein the side chain of said Lys residue is conjugated to a lipophilic substituent;
      and wherein the GlP analogue contains one and only one residue;
      or a pharmaceutically acceptable salt thereof.

Thus, a GlP analogue according to Formula II may be any of the following:

(SEQ ID NO: 116)
R¹-Tyr-X2-Glu-Gly-Thr-Phe-Ile-Ser-Asp-Tyr-Ser-X12-Glu-Leu-X15-X16-X17-X18-
X19-X20-X21-Phe-X23-X24-X25-Leu-X27-X28-X29-Lys-Gly-Y2-R²;
(SEQ ID NO: 103)
R¹-Tyr-X2-Glu-Gly-Thr-Phe-Ile-Ser-Asp-Tyr-Ser-X12-Glu-Leu-X15-X16-X17-X18-
X19-X20-X21-Phe-X23-X24-X25-Leu-X27-X28-X29-Gly-Pro-Ser-Ser-Gly-Ala-Pro-
Pro-Pro-Ser-Y2-R²;
(SEQ ID NO: 104)
R¹-Tyr-X2-Glu-Gly-Thr-Phe-Ile-Ser-Asp-Tyr-Ser-X12-Glu-Leu-X15-X16-X17-X18-
X19-X20-X21-Phe-X23-X24-X25-Leu-X27-X28-X29-Gly-Pro-Ser-Ser-Gly-Ala-Pro-
Pro-Ser-Y2-R²;
(SEQ ID NO: 105)
R¹-Tyr-X2-Glu-Gly-Thr-Phe-Ile-Ser-Asp-Tyr-Ser-X12-Glu-Leu-X15-X16-X17-X18-
X19-X20-X21-Phe-X23-X24-X25-Leu-X27-X28-X29-Pro-Ser-Ser-Gly-Ala-Pro-Pro-
Pro-Ser-Y2-R²;
(SEQ ID NO: 106)
R¹-Tyr-X2-Glu-Gly-Thr-Phe-Ile-Ser-Asp-Tyr-Ser-X12-Glu-Leu-X15-X16-X17-X18-
X19-X20-X21-Phe-X23-X24-X25-Leu-X27-X28-X29-Pro-Ser-Ser-Gly-Ala-Pro-Pro-
Ser-Y2-R²;
(SEQ ID NO: 107)
R¹-Tyr-X2-Glu-Gly-Thr-Phe-Ile-Ser-Asp-Tyr-Ser-X12-Glu-Leu-X15-X16-X17-X18-
X19-X20-X21-Phe-X23-X24-X25-Leu-X27-X28-X29-Gly-Lys-Lys-Asn-Asp-Trp-Lys-
His-Asn-Ile-Thr-Gln-Y2-R²;
or
(SEQ ID NO: 108)
R¹-Tyr-X2-Glu-Gly-Thr-Phe-Ile-Ser-Asp-Tyr-Ser-X12-Glu-Leu-X15-X16-X17-X18-
X19-X20-X21-Phe-X23-X24-X25-Leu-X27-X28-X29-Y2-R².

Combinations of residues which may be present at some of the variable positions of Formula II include:
Aib2, Lys12, Asp15, Lys20;
Aib2, Lys12, Asp15, Arg20;
Aib2, Asp15, Arg20;
Aib2, Ile12, Asp15, Arg20, Glu24;
Ile12, Asp15, Ile23;
Ile12, Asp15, Glu24;
Ile12, Asp15, Ala21;
Aib2, Lys12, Ala21, Glu24;
Aib2, Lys12, Asp15;
Aib2, Lys, 12, Asp15, Arg20, Gln29;
Aib2, Lys, 12, Asp15, Arg20, Gly29;
Aib2, Lys12, Asp15, Ile17, Arg20, Gly29;
Aib2, Asp15, Ile17, Lys20, Gly29;
DAla2, Asp15;
DAla2, Asp15, Ala28;
Aib2, Asp15, Ile17, Lys20, Ala28;
Asp15, Glu24;
Ala2, Lys12, Asp15, Lys20;
Ala2, Lys12, Asp15, Arg20;
Ala2, Asp15, Arg20;
Ala2, Ile12, Asp15, Arg20, Glu24;
Ala2, Ile12, Asp15, Ile23;
Ala2, Ile12, Asp15, Glu24;
Ala2, Ile12, Asp15, Ala21;
Ala2, Lys12, Ala21, Glu24;
Ala2, lys12, Asp15;
Ala2, Lys12, Asp15, Arg20, Gln29;
Ala2, Lys12, Asp15, Arg20, Gly29;
Ala2, Lys12, Asp15, Ile17, Arg20, Gly29;
Ala2, Asp15, Ile17, Lys20, Gly29;
Ala2, Asp15;
Ala2, Asp15, Ala28;
Ala2, Asp15, Ile17, Lys20, Ala28;
Gly2, Lys12, Asp15, Lys20;
Gly2, Lys12, Asp15, Arg20;
Gly2, Asp15, Arg20;
Gly2, Ile12, Asp15, Arg20, Glu24;
Gly2, Ile12, Asp15, Ile23;
Gly2, Ile12, Asp15, Glu24;
Gly2, Ile12, Asp15, Ala21;
Gly2, Lys12, Ala21, Glu24;
Gly2, Lys12, Asp15;
Gly2, Lys12, Asp 15, Arg20, Gln29;
Gly2, Lys 12, Asp15, Arg20, Gly29;
Gly2, Lys12, Asp15, Ile17, Arg20, Gly29;
Gly2, Asp15, Ile17, Lys20, Gly29;
Gly2, Asp15;
Gly2, Asp15, Ala28;
Gly2, Asp15, Ile17, Lys20, Ala28; or
Gly2, Asp15, Glu24.

The combination therapies of the invention can include a GlP analogue represented by the general Formula III:

(SEQ ID NO: 50)
R¹-Tyr-Aib-Glu-Gly-Thr-Phe-Ile-Ser-Asp-
Tyr-Ser-Ile-Glu-Leu-X15-X16-X17-X18-X19-
X20-X21-Phe-Val-X24-X25-Leu-Leu-Ala-X29-
Y1-Y2-R²
(III)

wherein
R¹ is H—, Ac or pGlu;
X15 is Asp or Glu;
X16 is Lys or ψ;
X17 is Ile or ψ;
X18 is His or Ala;
X19 is Gln or Ala;
X20 is Gln, Lys or Arg;
X21 is Ala, Asp or Glu;
X24 is Asn or Glu;
X25 is Tyr or Trp;
X28 is Ala, Ser or Arg;
X29 is Gln or is absent;
Y1 is Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), Gly-Lys-Lys-Asn-Asp-Trp-Lys-His-Asn-Ile-Thr-Gln (SEQ ID NO: 47) or absent;
Y2 is ψ or is absent;
R² is —NH₂ or —OH;
   wherein ψ is a residue independently selected from Lys, Arg, Orn and Cys and wherein the side chain of said residue is conjugated to a lipophilic substituent;
      and wherein the GlP analogue contains one and only one residue ψ;
      or a pharmaceutically acceptable salt thereof.

Thus, a GlP analogue according to Formula III may be any of the following:

(SEQ ID NO: 117)
R¹-Tyr-Aib-Glu-Gly-Thr-Phe-Ile-Ser-Asp-Tyr-
Ser-Ile-Glu-Leu-X15-X16-X17-X18-X19-X20-X21-
Phe-Val-X24-X25-Leu-Leu-Ala-X29-Lys-Gly-Y2-
R²;
(SEQ ID NO: 109)
R¹-Tyr-Aib-Glu-Gly-Thr-Phe-Ile-Ser-Asp-Tyr-
Ser-Ile-Glu-Leu-X15-X16-X17-X18-X19-X20-X21-
Phe-Val-X24-X25-Leu-Leu-Ala-X29-Gly-Pro-Ser-
Ser-Gly-Ala-Pro-Pro-Pro-Ser-Y2-R²;
(SEQ ID NO: 110)
R¹-Tyr-Aib-Glu-Gly-Thr-Phe-Ile-Ser-Asp-Tyr-
Ser-Ile-Glu-Leu-X15-X16-X17-X18-X19-X20-X21-
Phe-Val-X24-X25-Leu-Leu-Ala-X29-Gly-Pro-Ser-
Ser-Gly-Ala-Pro-Pro-Ser-Y2-R²;
(SEQ ID NO: 111)
R¹-Tyr-Aib-Glu-Gly-Thr-Phe-Ile-Ser-Asp-Tyr-
Ser-Ile-Glu-Leu-X15-X16-X17-X18-X19-X20-X21-
Phe-Val-X24-X25-Leu-Leu-Ala-X29-Pro-Ser-Ser-
Gly-Ala-Pro-Pro-Pro-Ser-Y2-R²;
(SEQ ID NO: 112)
R¹-Tyr-Aib-Glu-Gly-Thr-Phe-Ile-Ser-Asp-Tyr-
Ser-Ile-Glu-Leu-X15-X16-X17-X18-X19-X20-X21-
Phe-Val-X24-X25-Leu-Leu-Ala-X29-Pro-Ser-Ser-
Gly-Ala-Pro-Pro-Ser-Y2-R²;
(SEQ ID NO: 113)
R¹-Tyr-Aib-Glu-Gly-Thr-Phe-Ile-Ser-Asp-Tyr-
Ser-Ile-Glu-Leu-X15-X16-X17-X18-X19-X20-X21-
Phe-Val-X24-X25-Leu-Leu-Ala-X29-Gly-Lys-Lys-
Asn-Asp-Trp-Lys-His-Asn-Ile-Thr-Gln-Y2-R²;
or
(SEQ ID NO: 114)
R¹-Tyr-Aib-Glu-Gly-Thr-Phe-Ile-Ser-Asp-Tyr-
Ser-Ile-Glu-Leu-X15-X16-X17-X18-X19-X20-X21-
Phe-Val-X24-X25-Leu-Leu-Ala-X29-Y2-R².

Combinations of residues which may be present at some of the variable positions of Formula III include:
Asp15, Lys20;
Asp15, Arg20;
Asp15, Arg20, Glu24;
Asp15, Lys 16;
Asp15, Lys 16, Glu24;
Asp15, ψ16, Ala21;
Ala21, Glu24;
Asp15, Arg20, Gln29;
Asp15, Arg20, Gly29;
Asp15, Ile17, Arg20, Gly29;
Asp15, Ile17, Lys20, Gly29;
Asp15, Ala28;
Asp15, Ile17, Lys20, Ala28;
Asp15, Ile23, Glu24;
Asp15, ψ17, Lys20;
Asp15, ψ17, Arg20;
Asp15, ψ17, Arg20
Asp15, ψ17, Arg20, Glu24;
Asp15, Lys16, ψ17;
Asp15, Lys16, ψ17, Glu24;
Asp15, ψ17, Ala21;
Ala21, ψ17, Glu24;
Asp15, Asp15, ψ17, Arg20, Gln29;
Asp15, ψ17, Arg20, Gly29;
Asp15, Ile17, Arg20, Gly29;
Asp15, Ile17, Lys20, Gly29;
Asp15; ψ17;
Asp15, ψ17, Ala28;
Asp15, Ile17, Lys20, Ala28; or
Asp15, ψ17, Ile23, Glu24.

The GlP-analogue useful in the combination therapies of the invention may have the formula R1-Z—R2 where R1 and R2 are as defined above and Z has the sequence:

(SEQ ID NO: 51)
Y-Aib-EGTFISDYSIELDKYHQQDFVNWLLAQGPSSGAPPPS;
(SEQ ID NO: 52)
Y-Aib-EGTFISDYSIELDYIHQQDFVNWLLAQGPSSGAPPPS;
(SEQ ID NO: 53)
Y-Aib-EGTFISDYSIELEKYHQQDFVNWLLAQGPSSGAPPPS;
(SEQ ID NO: 54)
Y-Aib-EGTFISDYSIELDKIHQQDFVNWLLAQGPSSGAPPPSΨ;
(SEQ ID NO: 96)
Y-Aib-EGTFISDYSIELDKIHQQDFVNWLLAQGPSSGAPPPSΨ;
(SEQ ID NO: 55)
Y-Aib-EGTFISDYSIELDKIHQQDFVNWLLAQΨ;
(SEQ ID NO: 56)
Y-Aib-EGTFISDYSIELDKIHQQDFVNWLLAQKGΨ;
(SEQ ID NO: 57)
Y-Aib-EGTFISDYSIELDKYHQQDFVNYLLAQGPSSGAPPPS;
(SEQ ID NO: 58)
Y-Aib-EGTFISDYSIELDKYHQQDFVNWLLAQGPSSGAPPPS;
(SEQ ID NO: 59)
Y-Aib-EGTFISDYSIELDKYAAQDFVNWLLAQGPSSGAPPPS;
(SEQ ID NO: 60)
Y-Aib-EGTFISDYSIELEKYAAKEFVNWLLAQGPSSGAPPPS;
(SEQ ID NO: 61)
Y-Aib-EGTFISDYSIELEKYAQRAFVEWLLAQGPSSGAPPPS;
(SEQ ID NO: 62)
Y-Aib-EGTFISDYSIELEKIAQRAFVEWLLAQGPSSGAPPPSΨ;
(SEQ ID NO: 63)
Y-Aib-EGTFISDYSIELEKIAQRAFVEWLLAQΨ;
(SEQ ID NO: 64)
Y-Aib-EGTFISDYSIELDKYAAQDFVNWLLAGPSSGAPPPS;
(SEQ ID NO: 65)
Y-Aib-EGTFISDYSIELDKIAAQDFVNWLLAGPSSGAPPPSΨ;
(SEQ ID NO: 66)
Y-Aib-EGTFISDYSIELDKYAQRAFVEWLLAQGPSSGAPPPS;
(SEQ ID NO: 67)
Y-Aib-EGTFISDYSIELDKYAQRAFIEWLLAQGPSSGAPPPS;
(SEQ ID NO: 68)
Y-Aib-EGTFISDYSIELDKIAQRAFIEWLLAGPSSGAPPPSKΨ;
(SEQ ID NO: 69)
Y-Aib-EGTFISDYSIELDKIAQKEFIEWLLAGPSSGAPPPSKΨ;
(SEQ ID NO: 70)
Y-Aib-EGTFISDYSIELDKIAAQDFIEWLLAGPSSGAPPPSKΨ;
(SEQ ID NO: 71)
Y-Aib-EGTFISDYSIELDKIAAQDFVEWLLAGPSSGAPPPSKΨ;
(SEQ ID NO: 72)
Y-Aib-EGTFISDYSIELDKIAQRAFIEWLLAQGPSSGAPPPSKΨ;
(SEQ ID NO: 73)
Y-Aib-EGTFISDYSIELDKYAAQAFVNWLLAGPSSGAPPPS;
(SEQ ID NO: 74)
Y-Aib-EGTFISDYSIELDKYAAQDFVNWLLAAGPSSGAPPPS;
(SEQ ID NO: 75)
Y-Aib-EGTFISDYSIELDKYAAQDFINWLLAGPSSGAPPPS;
(SEQ ID NO: 76)
Y-Aib-EGTFISDYSIELDKYAAQDFIEWLLAGPSSGAPPPS;
(SEQ ID NO: 77)
Y-Aib-EGTFISDYSIELDKYAAQDFIEWLLAGPSSGAPPPS;
(SEQ ID NO: 78)
Y-Aib-EGTFISDYSIELDKIAQRAFIEWLLAQGPSSGAPPPS;
(SEQ ID NO: 79)
Y-Aib-EGTFISDYSYELDKIAQRAFIEWLLAQGPSSGAPPPS;
(SEQ ID NO: 80)
Y-DAla-EGTFISDYSIELDKYAQRAFIEWLLAQGPSSGAPPPS;
(SEQ ID NO: 81)
Y-DAla-EGTFISDYSIELDKIAAQDFIEWLLAGPSSGAPPPSKΨ;
(SEQ ID NO: 82)
Y-Aib-EGTFISDYSIELDKYAAQDFIEWLLAQGPSSGAPPPS;
(SEQ ID NO: 83)
Y-Aib-EGTFISDYSIELDKYAAQDFINWLLAQGPSSGAPPPS;
or
(SEQ ID NO: 84)
Y-Aib-EGTFISDYSIELDKYAAQAFIEWLLAQGPSSGAPPPS.

The GlP-analogue useful in the combination therapies of the invention may have the formula R1-Z—R2 where R1 and R2 are as defined above and Z has the sequence

(SEQ ID NO: 1)
Y-Aib-EGTFISDYSIELDK-K(Hexadecanoyl-isoGlu)-
HQQDFVNWLLAQGPSSGAPPPS;
(SEQ ID NO: 2)
Y-Aib-EGTFISDYSIELD-K(Hexadecanoyl-isoGlu)-
IHQQDFVNWLLAQGPSSGAPPPS;
(SEQ ID NO: 3)
Y-Aib-EGTFISDYSIELEK-K(Hexadecanoyl-isoGlu)-
HQQDFVNWLLAQGPSSGAPPPS;
(SEQ ID NO: 4)
Y-Aib-EGTFISDYSIELDKIHQQDFVNWLLAQGPSSGAPPPS-K([19-carboxy-
nonadecanoyl]-isoGlu-Peg3-Peg3)
(SEQ ID NO: 5)
Y-Aib-EGTFISDYSIELDKIHQQDFVNWLLAQGPSSGAPPPS-K(Hexadecanoyl-
isoGlu)
(SEQ ID NO: 6)
Y-Aib-EGTFISDYSIELDKIHQQDFVNWLLAQ-K(Hexadecanoyl-isoGlu)
(SEQ ID NO: 7)
Y-Aib-EGTFISDYSIELDKIHQQDFVNWLLAQKG-K(Hexadecanoyl-isoGlu)
(SEQ ID NO: 8)
Y-Aib-EGTFISDYSIELDK-K([19-carboxy-nonadecanoyl]-isoGlu-Peg3-Peg3)-
HQQDFVNYLLAQGPSSGAPPPS;
(SEQ ID NO: 9)
Y-Aib-EGTFISDYSIELDK-K([19-carboxy-nonadecanoyl]-isoGlu-Peg3-Peg3)-
HQQDFVNWLLAQGPSSGAPPPS;
(SEQ ID NO: 10)
Y-Aib-EGTFISDYSIELDK-K([19-carboxy-nonadecanoyl]-isoGlu-Peg3-Peg3)-
AAQDFVNWLLAQGPSSGAPPPS;
(SEQ ID NO: 11)
Y-Aib-EGTFISDYSIELEK-K([19-carboxy-nonadecanoyl]-isoGlu-Peg3-Peg3)-
AAKEFVNWLLAQGPSSGAPPPS;
(SEQ ID NO: 12)
Y-Aib-EGTFISDYSIELEK-K([19-carboxy-nonadecanoyl]-isoGlu-Peg3-Peg3)-
AQRAFVEWLLAQGPSSGAPPPS;
(SEQ ID NO: 13)
Y-Aib-EGTFISDYSIELEKIAQRAFVEWLLAQGPSSGAPPPS-K([19-carboxy-
nonadecanoyl]-isoGlu-Peg3-Peg3)
(SEQ ID NO: 14)
Y-Aib-EGTFISDYSIELEKIAQRAFVEWLLAQ-K([19-carboxy-nonadecanoyl]-isoGlu-
Peg3-Peg3)
(SEQ ID NO: 15)
Y-Aib-EGTFISDYSIELDK-K([19-carboxy-nonadecanoyl]-isoGlu-Peg3-Peg3)-
AAQDFVNWLLAGPSSGAPPPS;
(SEQ ID NO: 118)
H-Y-Aib-EGTFISDYSIELDK-K([19-carboxy-nonadecanoyl]-isoGlu-Peg3-Peg3)-
AAQDFVNWLLAGPSSGAPPPS;
(SEQ ID NO: 16)
Y-Aib-EGTFISDYSIELDKIAAQDFVNWLLAGPSSGAPPPS-K([19-carboxy-
nonadecanoyl]-isoGlu-Peg3-Peg3)
(SEQ ID NO: 17)
Y-Aib-EGTFISDYSIELDK-K([19-Carboxy-nonadecanoyl]-isoGlu-Peg3-Peg3)-
AQRAFVEWLLAQGPSSGAPPPS;
(SEQ ID NO: 18)
Y-Aib-EGTFISDYSIELDK-K([19-Carboxy-nonadecanoyl]-isoGlu-Peg3-Peg3)-
AQRAFIEWLLAQGPSSGAPPPS;
(SEQ ID NO: 19)
Y-Aib-EGTFISDYSIELDK-K((19-Carboxy-nonadecanoyl)-[(Piperazine-1-yl)-acetyl]-
Peg3-Peg3)-AQRAFIEWLLAQGPSSGAPPPS;
(SEQ ID NO: 20)
Y-Aib-EGTFISDYSIELDK-K((19-Carboxy-nonadecanoyl)-[(Piperazine-1-yl)-acetyl]-
Peg3-Peg3)-AQRAFVEWLLAQGPSSGAPPPS;
(SEQ ID NO: 21)
Y-Aib-EGTFISDYSIELDK-K((19-Carboxy-nonadecanoyl)-[(Piperazine-1-yl)-acetyl]-
Peg3-Peg3)-AQKEFVEWLLAAGPSSGAPPPS;
(SEQ ID NO: 22)
Y-Aib-EGTFISDYSIELDK-K([19-Carboxy-nonadecanoyl]-isoGlu-Peg3-Peg3)-
AQKEFVEWLLAAGPSSGAPPPS;
(SEQ ID NO: 23)
Y-Aib-EGTFISDYSIELDKIAQRAFIEWLLAGPSSGAPPPS-K([19-Carboxy-
nonadecanoyl]-isoGlu-Peg3-Peg3)
(SEQ ID NO: 24)
Y-Aib-EGTFISDYSIELDKIAQKEFIEWLLAGPSSGAPPPS-K([19-Carboxy-
nonadecanoyl]-isoGlu-Peg3-Peg3)
(SEQ ID NO: 25)
Y-Aib-EGTFISDYSIELDKIAAQDFIEWLLAGPSSGAPPPS-K([19-Carboxy-
nonadecanoyl]-isoGlu-Peg3-Peg3)
(SEQ ID NO: 26)
Y-Aib-EGTFISDYSIELDKIAAQDFIEWLLAGPSSGAPPPS-K((19-Carboxy-
nonadecanoyl)-[(Piperazine-1-yl)-acetyl]-Peg3-Peg3)
(SEQ ID NO: 27)
Y-Aib-EGTFISDYSIELDKIAAQDFVEWLLAGPSSGAPPPS-K([19-Carboxy-
nonadecanoyl]-isoGlu-Peg3-Peg3)
(SEQ ID NO: 28)
Y-Aib-EGTFISDYSIELDKIAQRAFIEWLLAQGPSSGAPPPS-K([19-Carboxy-
nonadecanoyl]-isoGlu-Peg3-Peg3)
(SEQ ID NO: 29)
Y-Aib-EGTFISDYSIELDK-K([19-Carboxy-nonadecanoyl]-isoGlu-Peg3-Peg3)-
AAQAFVNWLLAGPSSGAPPPS;
(SEQ ID NO: 30)
Y-Aib-EGTFISDYSIELDK-K([19-Carboxy-nonadecanoyl]-isoGlu-Peg3-Peg3)-
AAQDFVNWLLAAGPSSGAPPPS;
(SEQ ID NO: 31)
Y-Aib-EGTFISDYSIELDK-K([19-Carboxy-nonadecanoyl]-isoGlu-Peg3-Peg3)-
AAQDFINWLLAGPSSGAPPPS;
(SEQ ID NO: 32)
Y-Aib-EGTFISDYSIELDK-K([19-Carboxy-nonadecanoyl]-isoGlu-Peg3-Peg3)-
AAQDFIEWLLAGPSSGAPPPS;
(SEQ ID NO: 33)
Y-Aib-EGTFISDYSIELDK-K((19-Carboxy-nonadecanoyl)-[(Piperazine-1-yl)-acetyl]-
Peg3-Peg3)-AAQDFIEWLLAGPSSGAPPPS;
(SEQ ID NO: 34)
Y-Aib-EGTFISDYSIELD-K([19-Carboxy-nonadecanoyl]-isoGlu-Peg3-Peg3)-
IAQRAFIEWLLAQGPSSGAPPPS;
(SEQ ID NO: 35)
Y-Aib-EGTFISDYS-K([19-Carboxy-nonadecanoyl]-isoGlu-Peg3-Peg3)-
ELDKIAQRAFIEWLLAQGPSSGAPPPS;
(SEQ ID NO: 36)
Y-DAla-EGTFISDYSIELDK-K([19-Carboxy-nonadecanoyl]-isoGlu-Peg3-Peg3)-
AQRAFIEWLLAQGPSSGAPPPS;
(SEQ ID NO: 37)
Y-DAla-EGTFISDYSIELDKIAAQDFIEWLLAGPSSGAPPPS-K((19-Carboxy-
nonadecanoyl)-[(Piperazine-1-yl)-acetyl]-Peg3-Peg3)
(SEQ ID NO: 38)
Y-Aib-EGTFISDYSIELDK-K([19-Carboxy-nonadecanoyl]-isoGlu-Peg3-Peg3)-
AAQDFIEWLLAQGPSSGAPPPS;
(SEQ ID NO: 39)
Y-Aib-EGTFISDYSIELDK-K([19-Carboxy-nonadecanoyl]-isoGlu-Peg3-Peg3)-
AAQDFINWLLAQGPSSGAPPPS;
(SEQ ID NO: 40)
Y-Aib-EGTFISDYSIELDK-K([19-Carboxy-nonadecanoyl]-isoGlu-Peg3-Peg3)-
AAQAFIEWLLAQGPSSGAPPPS;
or
(SEQ ID NO: 41)
Y-Aib-EGTFISDYSIELDK-K((19-Carboxy-nonadecanoyl)-[(Piperazine-1-yl)-acetyl]-
Peg3-Peg3)-AAQAFIEWLLAQGPSSGAPPPS.

The GlP-analogue used in the combination therapy may be any of the following, or a pharmaceutically acceptable salt thereof:

(SEQ ID NO: 1)
H-Y-Aib-EGTFISDYSIELDK-K(Hexadecanoyl-isoGlu)-
HQQDFVNWLLAQGPSSGAPPPS-NH₂;
(Compound 1)
(SEQ ID NO: 2)
H-Y-Aib-EGTFISDYSIELD-K(Hexadecanoyl-isoGlu)-
IHQQDFVNWLLAQGPSSGAPPPS-NH₂;
(Compound 2)
(SEQ ID NO: 3)
H-Y-Aib-EGTFISDYSIELEK-K(Hexadecanoyl-isoGlu)-
HQQDFVNWLLAQGPSSGAPPPS-NH₂;
(Compound 3)
(SEQ ID NO: 4)
H-Y-Aib-EGTFISDYSIELDKIHQQDFVNWLLAQGPSSGAPPPS-K([19-carboxy-
nonadecanoyl]-isoGlu-Peg3-Peg3)-NH₂;
(Compound 4)
(SEQ ID NO: 5)
H-Y-Aib-EGTFISDYSIELDKIHQQDFVNWLLAQGPSSGAPPPS-K(Hexadecanoyl-
isoGlu)-NH₂;
(Compound 5)
(SEQ ID NO: 6)
H-Y-Aib-EGTFISDYSIELDKIHQQDFVNWLLAQ-K(Hexadecanoyl-isoGlu)-NH₂;
(Compound 6)
(SEQ ID NO: 7)
H-Y-Aib-EGTFISDYSIELDKIHQQDFVNWLLAQKG-K(Hexadecanoyl-isoGlu)-NH₂;
(Compound 7)
(SEQ ID NO: 8)
H-Y-Aib-EGTFISDYSIELDK-K([19-carboxy-nonadecanoyl]-isoGlu-Peg3-Peg3)-
HQQDFVNYLLAQGPSSGAPPPS-NH₂;
(Compound 8)
(SEQ ID NO: 9)
H-Y-Aib-EGTFISDYSIELDK-K([19-carboxy-nonadecanoyl]-isoGlu-Peg3-Peg3)-
HQQDFVNWLLAQGPSSGAPPPS-NH₂;
(Compound 9)
(SEQ ID NO: 10)
H-Y-Aib-EGTFISDYSIELDK-K([19-carboxy-nonadecanoyl]-isoGlu-Peg3-Peg3)-
AAQDFVNWLLAQGPSSGAPPPS-NH₂;
(Compound 10)
(SEQ ID NO: 11)
H-Y-Aib-EGTFISDYSIELEK-K([19-carboxy-nonadecanoyl]-isoGlu-Peg3-Peg3)-
AAKEFVNWLLAQGPSSGAPPPS-NH₂;
(Compound 11)
(SEQ ID NO: 12)
H-Y-Aib-EGTFISDYSIELEK-K([19-carboxy-nonadecanoyl]-isoGlu-Peg3-Peg3)-
AQRAFVEWLLAQGPSSGAPPPS-NH₂;
(Compound 12)
(SEQ ID NO: 13)
H-Y-Aib-EGTFISDYSIELEKIAQRAFVEWLLAQGPSSGAPPPS-K([19-carboxy-
nonadecanoyl]-isoGlu-Peg3-Peg3)-NH₂;
(Compound 13)
(SEQ ID NO: 14)
H-Y-Aib-EGTFISDYSIELEKIAQRAFVEWLLAQ-K([19-carboxy-nonadecanoyl]-
isoGlu-Peg3-Peg3)-NH₂;
(Compound 14)
(SEQ ID NO: 15)
H-Y-Aib-EGTFISDYSIELDK-K([19-carboxy-nonadecanoyl]-isoGlu-Peg3-Peg3)-
AAQDFVNWLLAGPSSGAPPPS-NH₂;
(Compound 15)
(SEQ ID NO: 16)
H-Y-Aib-EGTFISDYSIELDKIAAQDFVNWLLAGPSSGAPPPS-K([19-carboxy-
nonadecanoyl]-isoGlu-Peg3-Peg3)-NH₂;
(Compound 16)
(SEQ ID NO: 17)
H-Y-Aib-EGTFISDYSIELDK-K([19-Carboxy-nonadecanoyl]-isoGlu-Peg3-Peg3)-
AQRAFVEWLLAQGPSSGAPPPS-NH₂;
(Compound 17)
(SEQ ID NO: 18)
H-Y-Aib-EGTFISDYSIELDK-K([19-Carboxy-nonadecanoyl]-isoGlu-Peg3-Peg3)-
AQRAFIEWLLAQGPSSGAPPPS-NH₂;
(Compound 18)
(SEQ ID NO: 19)
H-Y-Aib-EGTFISDYSIELDK-K((19-Carboxy-nonadecanoyl)-[(Piperazine-1-yl)-acetyl]-
Peg3-Peg3)-AQRAFIEWLLAQGPSSGAPPPS-NH₂;
(Compound 19)
(SEQ ID NO: 20)
H-Y-Aib-EGTFISDYSIELDK-K((19-Carboxy-nonadecanoyl)-[(Piperazine-1-yl)-acetyl]-
Peg3-Peg3)-AQRAFVEWLLAQGPSSGAPPPS-NH₂;
(Compound 20)
(SEQ ID NO: 21)
H-Y-Aib-EGTFISDYSIELDK-K((19-Carboxy-nonadecanoyl)-[(Piperazine-1-yl)-acetyl]-
Peg3-Peg3)-AQKEFVEWLLAAGPSSGAPPPS-NH₂;
(Compound 21)
(SEQ ID NO: 22)
H-Y-Aib-EGTFISDYSIELDK-K([19-Carboxy-nonadecanoyl]-isoGlu-Peg3-Peg3)-
AQKEFVEWLLAAGPSSGAPPPS-NH₂;
(Compound 22)
(SEQ ID NO: 23)
H-Y-Aib-EGTFISDYSIELDKIAQRAFIEWLLAGPSSGAPPPS-K([19-Carboxy-
nonadecanoyl]-isoGlu-Peg3-Peg3)-NH₂;
(Compound 23)
(SEQ ID NO: 24)
H-Y-Aib-EGTFISDYSIELDKIAQKEFIEWLLAGPSSGAPPPS-K([19-Carboxy-
nonadecanoyl]-isoGlu-Peg3-Peg3)-NH₂;
(Compound 24)
(SEQ ID NO: 25)
H-Y-Aib-EGTFISDYSIELDKIAAQDFIEWLLAGPSSGAPPPS-K([19-Carboxy-
nonadecanoyl]-isoGlu-Peg3-Peg3)-NH₂;
(Compound 25)
(SEQ ID NO: 26)
H-Y-Aib-EGTFISDYSIELDKIAAQDFIEWLLAGPSSGAPPPS-K((19-Carboxy-
nonadecanoyl)-[(Piperazine-1-yl)-acetyl]-Peg3-Peg3)-NH₂;
(Compound 26)
(SEQ ID NO: 27)
H-Y-Aib-EGTFISDYSIELDKIAAQDFVEWLLAGPSSGAPPPS-K([19-Carboxy-
nonadecanoyl]-isoGlu-Peg3-Peg3)-NH₂;
(Compound 27)
(SEQ ID NO: 28)
H-Y-Aib-EGTFISDYSIELDKIAQRAFIEWLLAQGPSSGAPPPS-K([19-Carboxy-
nonadecanoyl]-isoGlu-Peg3-Peg3)-NH₂;
(Compound 28)
(SEQ ID NO: 29)
H-Y-Aib-EGTFISDYSIELDK-K([19-Carboxy-nonadecanoyl]-isoGlu-Peg3-Peg3)-
AAQAFVNWLLAGPSSGAPPPS-NH₂;
(Compound 29)
(SEQ ID NO: 30)
H-Y-Aib-EGTFISDYSIELDK-K([19-Carboxy-nonadecanoyl]-isoGlu-Peg3-Peg3)-
AAQDFVNWLLAAGPSSGAPPPS-NH₂;
(Compound 30)
(SEQ ID NO: 31)
H-Y-Aib-EGTFISDYSIELDK-K([19-Carboxy-nonadecanoyl]-isoGlu-Peg3-Peg3)-
AAQDFINWLLAGPSSGAPPPS-NH₂;
(Compound 31)
(SEQ ID NO: 32)
H-Y-Aib-EGTFISDYSIELDK-K([19-Carboxy-nonadecanoyl]-isoGlu-Peg3-Peg3)-
AAQDFIEWLLAGPSSGAPPPS-NH₂;
(Compound 32)
(SEQ ID NO: 33)
H-Y-Aib-EGTFISDYSIELDK-K((19-Carboxy-nonadecanoyl)-[(Piperazine-1-yl)-acetyl]-
Peg3-Peg3)-AAQDFIEWLLAGPSSGAPPPS-NH₂;
(Compound 33)
(SEQ ID NO: 34)
H-Y-Aib-EGTFISDYSIELD-K([19-Carboxy-nonadecanoyl]-isoGlu-Peg3-Peg3)-
IAQRAFIEWLLAQGPSSGAPPPS-NH₂;
(Compound 34)
(SEQ ID NO: 35)
H-Y-Aib-EGTFISDYS-K([19-Carboxy-nonadecanoyl]-isoGlu-Peg3-Peg3)-
ELDKIAQRAFIEWLLAQGPSSGAPPPS-NH₂; (Compound 35)
(SEQ ID NO: 36)
H-Y-DAla-EGTFISDYSIELDK-K([19-Carboxy-nonadecanoyl]-isoGlu-Peg3-Peg3)-
AQRAFIEWLLAQGPSSGAPPPS-NH₂;
(Compound 36)
(SEQ ID NO: 37)
H-Y-DAla-EGTFISDYSIELDKIAAQDFIEWLLAGPSSGAPPPS-K((19-Carboxy-
nonadecanoyl)-[(Piperazine-1-yl)-acetyl]-Peg3-Peg3)-NH₂;
(Compound 37)
(SEQ ID NO: 38)
H-Y-Aib-EGTFISDYSIELDK-K([19-Carboxy-nonadecanoyl]-isoGlu-Peg3-Peg3)-
AAQDFIEWLLAQGPSSGAPPPS-NH₂;
(Compound 38)
(SEQ ID NO: 39)
H-Y-Aib-EGTFISDYSIELDK-K([19-Carboxy-nonadecanoyl]-isoGlu-Peg3-Peg3)-
AAQDFINWLLAQGPSSGAPPPS-NH₂;
(Compound 39)
(SEQ ID NO: 40)
H-Y-Aib-EGTFISDYSIELDK-K([19-Carboxy-nonadecanoyl]-isoGlu-Peg3-Peg3)-
AAQAFIEWLLAQGPSSGAPPPS-NH₂;
(Compound 40)
or
(SEQ ID NO: 41)
H-Y-Aib-EGTFISDYSIELDK-K((19-Carboxy-nonadecanoyl)-[(Piperazine-1-yl)-acetyl]-
Peg3-Peg3)-AAQAFIEWLLAQGPSSGAPPPS-NH₂.
(Compound 41)

For use in the combination therapies of the invention, compound 41, or a salt thereof, is the preferred GlP agonist. Compound 41 is also described in WO 2016/066744 as compound 41.

a. Route of Administration of GlP Agonist

The administration to a subject of the GlP agonist may be by any mode of administration common or standard in the art, e.g. oral, intravenous, intramuscular, subcutaneous, sublingual, intranasal, intradermal, suppository routes or implanting. The GlP agonist may be administered by one or more of the following routes of administration: injection (including subcutaneous injection, intramuscular injection, intravenous injection, epidural injection and intraperitoneal injection), infusion pump (such as subcutaneous infusion pump), oral, sublingual, rectal, vaginal, parenteral, topical, dermal (such as by transdermal patch), nasal (such as by nasal spray) and pulmonary spray.

The GlP agonist may be administered by injection. For certain embodiments, the GlP agonist may be administered by subcutaneous injection.

b. Dose of GlP Agonist

The GlP agonist may be administered at a dose of up to about 100 mg, such as up to about 90 mg, up to about 80 mg, up to about 70 mg, up to about 60 mg, up to about 50 mg, up to about 40 mg, up to about 30 mg, up to about 20 mg, up to about 15 mg, up to about 12.5 mg, up to about 10 mg, up to about 7.5 mg, up to about 5 mg, up to about 2.5 mg, up to about 2 mg, up to about 1 mg, up to about 0.5 mg or up to about 0.1 mg.

The GlP agonist may be administered at a dose of from about 0.1 mg to about 100 mg, from about 0.5 mg to about 100 mg, from about 1 mg to about 100 mg, from about 2.5 mg to about 100 mg, from about 5 mg to about 100 mg, from about 7.5 mg to about 100 mg, from about 10 mg to about 100 mg, from about 12.5 mg to about 100 mg, from about 15 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg.

The GlP agonist may be administered at a dose of from about 0.1 mg to about 80 mg, from about 0.5 mg to about 80 mg, from about 1 mg to about 80 mg, from about 2.5 mg to about 80 mg, from about 5 mg to about 80 mg, from about 7.5 mg to about 80 mg, from about 10 mg to about 80 mg, from about 12.5 mg to about 80 mg, from about 15 mg to about 80 mg, from about 20 mg to about 80 mg, from about 30 mg to about 80 mg, from about 40 mg to about 80 mg, from about 50 mg to about 80 mg, from about 60 mg to about 80 mg, or from about 70 mg to about 80 mg.

The GlP agonist may be administered at a dose of from about 0.1 mg to about 50 mg, from about 0.5 mg to about 50 mg, from about 1 mg to about 50 mg, from about 2.5 mg to about 50 mg, from about 5 mg to about 50 mg, from about 7.5 mg to about 50 mg, from about 10 mg to about 50 mg, from about 12.5 mg to about 50 mg, from about 15 mg to about 50 mg, from about 20 mg to about 50 mg, from about 30 mg to about 50 mg, or from about 40 mg to about 50 mg.

The GlP agonist may be administered at a dose of from about 0.1 mg to about 15 mg, from about 0.5 mg to about 15 mg, from about 1 mg to about 15 mg, from about 2.5 mg to about 15 mg, from about 5 mg to about 15 mg, from about 7.5 mg to about 15 mg, from about 10 mg to about 15 mg, or from about 12.5 mg to about 15 mg.

The GlP agonist may be administered at a dose of from about 0.5 mg to about 10 mg, about 1 mg to about 10 mg, from about 2.5 mg to about 10 mg, from about 5 mg to about 10 mg, or from about 7.5 mg to about 10 mg.

The GlP agonist may be administered at a dose of from about 1 mg to about 5 mg, or from about 2.5 mg to about 5 mg.

The GlP agonist may be administered at a dose of about 100 mg, about 90 mg, about 80 mg, about 70 mg, about 60 mg, about 50 mg, about 40 mg, about 30 mg, about 20 mg, about 15 mg, about 12.5 mg, about 10 mg, about 7.5 mg, about 5 mg, about 2.5 mg, or about 1 mg.

The GlP agonist may be administered to the subject at a dose of up to about 500 nmol/kg body weight, such as up to about 400 nmol/kg body weight, or up to about 300 nmol/kg body weight.

The GlP agonist may be administered to the subject at a dose of from about 10 to about 500 nmol/kg body weight, such as from about 50 to about 500 nmol/kg body weight, from about 100 to about 500 nmol/kg body weight, or from about 200 to about 500 nmol/kg body weight. The GlP agonist may be administered to the subject at a dose of from about 10 to about 400 nmol/kg body weight, such as from about 50 to about 400 nmol/kg body weight, from about 100 to about 400 nmol/kg body weigh, or from about 200 to about 400 nmol/kg body weight. The GlP agonist may be administered to the subject at a dose of from about 250 to about 350 nmol/kg body weight. For certain embodiments, the GlP agonist may be administered at a dose of about 300 nmol/kg body weight.

c. Timing of Administration of GlP Agonist

The GlP agonist may be administered to the subject at least once, twice, or three times daily. The GlP agonist may be administered to the subject once every 2 days, every 3 days, every 4 days, every 5 days, every 6 days, every 7 days, every 8 days, every 9 days, every 10 days, every 11 days, every 12 days, every 13 days, every 14 days or more such as every 28 days or every month. For certain embodiments, the GLP-1 agonist of step (i) and/or step (ii) may be administered to the subject once a day (i.e. daily or once daily). For certain embodiments, the GLP-1 agonist of step (i) and step (ii) may be administered to the subject once a week (i.e weekly or once weekly).

The GlP agonist may be administered at the same time or at a different time as the administration of the GLP-1 agonist of step (ii).

d. Lipophilic Groups

The GlP analogue used in the methods of the invention can comprise a residue Y, i.e. a residue selected from Lys, Arg, Orn and Cys in which the side chain is conjugated to a lipophilic substituent.

Without wishing to be bound by any particular theory, it is thought that the substituent binds plasma proteins (e.g. albumin) in the blood stream, thus shielding the compounds of the invention from enzymatic degradation and thereby enhancing the half-life of the compounds. It may also modulate the potency of the compound, e.g. with respect to the GlP receptor.

The substituent is conjugated to the functional group at the distal end of the side chain from the alpha-carbon. The normal ability of the Lys, Arg, Orn or Cys side chain to participate in interactions mediated by that functional group (e.g. intra- and inter-molecular interactions) may therefore be reduced or completely eliminated by the presence of the substituent. Thus, the overall properties of the compound may be relatively insensitive to changes in the actual amino acid present as residue ψ. Consequently, it is believed that any of the residues Lys, Arg, Orn and Cys may be present at any position where ψ is permitted. However, in certain embodiments, it may be advantageous that the amino acid component of ψ is Lys.

Thus, ψ is a residue of Lys, Arg, Orn or Cys in which the side chain is conjugated to a substituent having the formula —Z¹ or —Z²-Z¹
—Z¹ is a fatty chain having at a terminus a connection —X— to ψ or to Z²;
wherein
—X— is a bond, —CO—, —SO—, or —SO₂—;
and, optionally, Z¹ has a polar group at the end of the chain distal from connection —X—; said polar group comprising a carboxylic acid or a carboxylic acid bioisostere, a phosphonic acid, or a sulfonic acid group;
and wherein —Z²—, if present, is a spacer of formula:

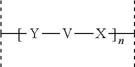

connecting Z¹ to ψ;
wherein:
each Y is independently —NH, —NR, —S or —O, where R is alkyl, a protecting group or forms a linkage to another part of the spacer Z²;
each X is independently a bond, CO—, SO—, or SO₂—;
with the proviso that when Y is —S, the X to which it is bound is a bond;
each V is independently a bivalent organic moiety linking Y and X;
and n is 1-10.

### The Group Z¹

Z¹ is a fatty chain having a connection to Y or to Z², referred to herein as —X—. —X— may be, for example, a bond, acyl (—CO—), sulfinyl (—SO—), or sulfonyl (—SO₂—). When Z¹ is bound directly to ψ, that is, when Z² is not present, preferably —X— is acyl (—CO—), sulfinyl (—SO—), or sulfonyl (—SO₂—). Most preferably, —X— is acyl (—CO—).

Z¹ may further have a polar group, said polar group being located at the end of the chain distal from the connection —X—. In other words, the connection is located at the @-position with respect to the polar group. The polar group may be bound directly to the terminus of the fatty chain, or may be bound via a linker.

Preferably, the polar group is an acidic or weakly acid group, for example a carboxylic acid or a carboxylic acid bioisostere, a phosphonate, or a sulfonate. The polar group may have a pKa of between −2 and 12 in water, more preferably between 1 and 7, more preferably between 3 and 6. Certain preferred polar groups have a pKa of between 4 and 5. For example, and not by way of limitation, the polar group may comprise a carboxylic acid (—COOH) or a carboxylic acid bioisostere, a phosphonic acid (—P(O)(OH)₂), or a sulfonic acid (—SO₂OH) group.

Preferably the polar group, if present, comprises a carboxylic acid or carboxylic acid bioisostere. Suitable carboxylic acid bioisosteres are known in the art. Preferably the bioisostere has a proton having a pKa similar to the corresponding carboxylic acid. Examples of suitable bioisoteres may include, not by way of limitation, tetrazole, acylsulfomides, acylhydroxylamine, and squaric acid derivatives, as shown below (--- indicates the point of attachment):

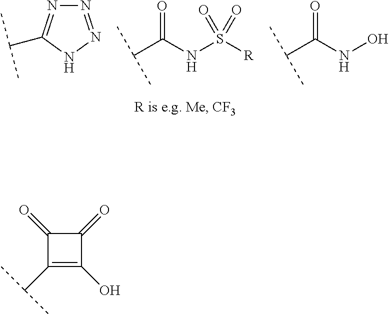

Fatty chain as used herein refers to a moiety comprising a chain of carbon atoms, the carbon atoms being predominantly substituted with hydrogen or hydrogen-like atoms, for example, a hydrocarbon chain. Such fatty chains are often referred to as lipophilic, although it will be appreciated that substitution may alter the lipophilic properties of the overall molecule.

The fatty chain may by aliphatic. It may be entirely saturated or may include one or more double or triple bonds. Each double bond, if present, may be in the E or Z configuration. The fatty chain may also have one or more cycloalkylene or heterocycloalkylene moieties in its length, and additionally or alternatively may have one or more arylene or heteroarylene moieties in its length. For example, the fatty chain may incorporate a phenylene or piperazinylene moiety in its length as, for example, shown below (wherein --- represents the points of attachment within the chain).

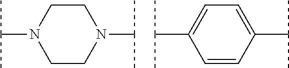

The fatty chain may be derived from a fatty acid, for example, it may be derived from a medium-chain fatty acid (MCFA) with an aliphatic tail of 6-12 carbon atoms, a long-chain fatty acid (LCFA) with an aliphatic tail of 13-21 carbon atoms, or a very long-chain fatty acid (LCFA) with an aliphatic tail of 22 carbon atoms or more. Examples of linear saturated fatty acids from which suitable fatty chains may be derived include tridecylic (tridecanoic) acid, myristic (tetradecanoic) acid, pentadecylic (pentadecanoic) acid, palmitic (hexadecanoic) acid, and margaric (heptadecanoic) acid. Examples of linear unsaturated fatty acids from which suitable fatty chains may be derived include myristoleic acid, palmitoleic acid, sapienic acid and oleic acid.

The fatty chain may be connected to ψ or to Z² by an amide linkage, a sulfinamide linkage, a sulfonamide linkage, or by an ester linkage, or by an ether, thioether or amine linkage. Accordingly, the fatty chain may have, a bond to Y or to Z² or an acyl (—CO—), sulfinyl (—SO—), or sulfonyl (—SO₂—) group. Preferably, the fatty chain has a terminus having an acyl (—CO—) group and is connected to ψ or Z² by an amide or ester linkage.

In some embodiments, Z¹ is a group of formula:

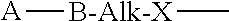

wherein
A is hydrogen or a carboxylic acid, a carboxylic acid bioisostere, a phosphonic acid, or a sulfonic acid group;
B is a bond or a linker;
X is a bond, acyl (—CO—), sulfinyl (—SO—), or sulfonyl (—SO₂—); and
Alk is a fatty chain that may be optionally substituted with one or more substituents. The fatty chain is preferably 6 to 28 carbon atoms in length (e.g. a C₆₋₂₈alkylene), more preferably, 12 to 26 carbons in length (e.g. a C₁₂₋₂₆alkylene), more preferably, 16 to 22 carbons in length (e.g. C₁₆₋₂₂alkylene), and may be saturated or unsaturated. Preferably, Alk is saturated, that is, preferably Alk is alkylene.

Optional substituents on the fatty chain may be independently selected from fluoro, C₁₋₄alkyl, preferably methyl; trifluoromethyl, hydroxymethyl, amino, hydroxyl, C₁₋₄alkoxy, preferably methoxy; oxo, and carboxyl, and may be independently located at any point along the chain. In some embodiments, each optional substituent is selected from fluoro, methyl, and hydroxyl. Where more than one substituent is present, substituents may be the same or different. Preferably, the number of substituents is 0 to 3; more preferably the fatty chain is unsubstituted.

B may be a bond or a linker. When B is a linker, it may be a cycloalkylene, heterocycloalkylene, C₆arylene, or C₅₋₆heteroarylene, or C₆arylene-O— or C₅₋₆heteroarylene-O—.

When B is phenylene it may, for example, be selected from 1,2-phenylene, 1,3-phenylene, 1,4-phenylene, preferably 1,4-phenylene (so that A-B- is a 4-benzoic acid substituent or 4-benzoic acid bioisostere). When B is phenylene-O—, it may, for example, be selected from 1,2-phenylene-O—, 1,3-phenylene-O—, 1,4-phenylene-O—, preferably 1,4-phenylene-O. Each phenylene of B may be optionally substituted with one or more substituents selected from fluoro, methyl, trifluoromethyl, amino, hydroxyl, and C₁₋₄alkoxy, preferably methoxy. It will be appreciated that substituent identity and position may be selected to subtly alter the pKa of the polar group. Suitable inductively or mesomerically electron-withdrawing or donating groups and their positional effects are known in the art. In some embodiments, B may be C₅₋₆heteroarylene, for example, pyridinylene or thiofuranylene, and may be optionally substituted as described.

For example, in some embodiments, A-B- may be selected from:

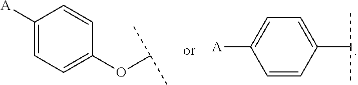

Preferably, A is H— or HOOC— and B is a bond.

It will be understood that when A is hydrogen, B is a bond and Alk is unsubstituted alkylene, A-B-Alk- is an alkyl chain of formula H₃C—(CH₂)ₙ—.

In some embodiments, Z¹ is an acyl group of formula:

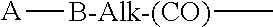

or a sulfonyl group of formula:

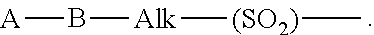

Preferably, Z¹ is an acyl group of formula:

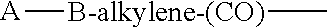

where A and B are as defined above.

In some embodiments, A is —COOH and B is a bond. Accordingly, certain preferred Z¹ are derived from long-chain saturated α,ω-dicarboxylic acids of formula HOOC—(CH₂)₁₂₋₂₂—COOH, preferably, long-chain saturated α,ω-dicarboxylic acids having an even number of carbon atoms in the aliphatic chain. In some other embodiments, A is H and B is a bond. Accordingly, certain preferred Z¹ are derived from long-chain saturated carboxylic acids of formula HOOC—(CH₂)₁₂₋₂₂—CH₃, preferably, long-chain saturated carboxylic acids having an even number of carbon atoms in the aliphatic chain.

For example, and not by way of limitation, Z¹ may be:
A-B-C₁₆₋₂₀alkylene-(CO)— wherein A is H or —COOH and B is a bond, for example:
17-carboxy-heptadecanoyl HOOC—(CH₂)₁₆—(CO)—;
19-carboxy-nonadecanoyl HOOC—(CH₂)₁₈—(CO)—;
Octadecanoyl H₃C—(CH₂)₁₆—(CO)—;
Eicosanoyl H₃C—(CH₂)₁₈—(CO)—;

The carboxylic acid group, if present, may be replaced by a bioisotere as detailed herein.

### The Group Z²

Z² is an optional spacer that connects Z¹ to the side chain of the amino acid component of ψ. At its most general, Z², if present, is a spacer bound at one terminus by Y, which may be a nitrogen, oxygen or sulfur atom, and at the other terminus by X, which may be a bond or an acyl (—CO—), sulfinyl (—SO—), sulfonyl (—SO₂—) or absent. Accordingly, Z² may be a spacer of formula (--- indicate points of attachment):

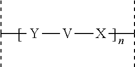

wherein:
Y may be —NH, —NR, —S or —O, where R may be alkyl, a protecting group or may form a linkage to another part of the spacer, with the remaining valency forming a linkage to Z¹;
X may be a bond, CO—, SO—, or SO₂—, with the remaining valency forming a linkage to the side chain of the amino acid component of ψ;
V is a bivalent organic moiety linking Y and X;
and n may be 1, 2, 3, 4, 5, 6, 7, 8, 9 or 10. Where n is 2 or more, each Y, V, and X is independent of every other Y, V, and X.

Accordingly, Z² may be bound at each side by amide, sulfinamide, sulfonamide, or ester linkages or by amino, ether, or thioether linkages depending upon the nature of Y and X and the corresponding linking groups on Z¹ and the side chain. Where n is 2 or greater, each V may also be bound to each adjacent V by linkages as described. Preferably, linkages are amides, esters or sulfonamides, most preferably amides. Accordingly, in some embodiments, each Y is —NH or —NR and each X is CO— or SO₂—. Most preferably, —X— is acyl (—CO—).

In some embodiments, Z² is a spacer of formula -S_{A}-, -S_{B}-, -S_{A}-S_{B}- or -S_{B}-S_{A}-, wherein S_{A} and S_{B} are as defined below.

In some embodiments, Z² is selected from -S_{A}-or -S_{B}-S_{A}-, that is, [side chain]-Z²Z¹ is [side chain]-S_{A}—Z¹ or [side chain]-S_{B}-S_{A}—Z¹.

### The Group S_{A}

S_{A} may be a single amino acid residue or a residue of an amino acid derivative, especially an amino acid derivative residue having a sulfinyl or sulfonyl in place of the carboxy moiety at the C terminus. Additionally or alternatively, the single amino acid residue may have an oxygen or sulfur atom in place of the nitrogen atom at the N terminus.

S_{A} may be or may comprise a nitrogen-containing heterocycle, said nitrogen-containing heterocycle being bound within the lipophilic group at one end via a bond, a carboxy, a sulfinyl, or a sulfonyl group and at the other via a ring nitrogen atom. For example, S_{A} may comprise a piperazine ring.

Suitably, S_{A} is a 5-8-membered heterocycle having 1 or 2 nitrogen atoms and substituted with an X group, where X is a bond, CO—, SO—, or SO₂—, and where L, if present, is C₁₋₄alkylene (- denotes a point of attachment within the lipophilic group).

Preferably, S_{A} is a 6-membered heterocycle having 1 or 2 nitrogen atoms, preferably 2, and substituted with a —CH₂CO—, —CH₂SO—, or —CH₂SO₂— group.

For example, S_{A} may be:

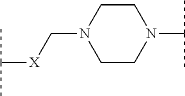

For example, S_{A} may be:

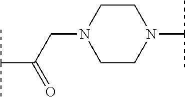

(referred to herein as piperazine-1-yl-acetyl).

Preferably, S_{A} is a single amino acid residue or piperazine-1-yl-acetyl. More preferably S_{A} is a single amino acid residue.

In some embodiments, the amino acid may be selected from γ-Glu, α-Glu, α-Asp, β-Asp, Ala, β-Ala (3-aminopropanoic acid), Dapa (2,3-diaminopropanoic acid), Dab (2,4-diaminobutanoic acid), and Gaba (4-aminobutanoic acid). It will be understood that where more than one carboxylic acid or amino moiety is present, connection may be at any moiety as appropriate. Any carboxylic acid or amino resides not bound within the residue may be free, that is, present as a free carboxylic acid or primary amine, or may be derivatised. Suitable derivatisation is known in the art. For example, carboxylic acid moieties may be present in S_{A} amino acid residues as esters, for example, as methyl esters. Amino moieties may be present as alkylated amines, for example, methylated, or may be protected as amide or carbamate moieties. Other suitable amino acids include β-Ala (3-aminopropanoic acid) and Gaba (4-aminobutanoic acid) and similar w amino acids.

It will be understood that amino acids may be D or L, or a racemic or enantioenriched mixture. In some embodiments, the amino acid is an L-amino acid. In some embodiments, the amino acid is a D-amino acid.

In some preferred embodiments, S_{A} has a carboxylic acid substituent, with γ-Glu, α-Glu, α-Asp, and β-Asp, and sulfinyl and sulfonyl derivatives thereof, being preferred. Accordingly, in some embodiments, the amino acid residue is:

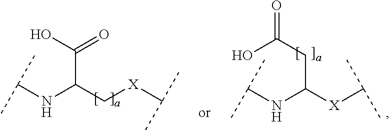

where —X— is —CO—,—SO—, —SO₂—, preferably —CO—, and a is 1 or 2, preferably 2. In some embodiments, the carboxylic acid is an ester, and the amino acid residue is:

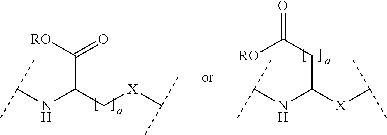

where —X— is —CO—, —SO—, —SO₂—, preferably —CO—, and a is 1 or 2, preferably 2, and R is C₁₋₄alkyl or C₆aryl. Preferably R is C₁₋₄alkyl, preferably methyl or ethyl, more preferably ethyl.

A preferred S_{A} group bearing a carboxylic acid is γ-Glu.

Preferably, S_{A} is selected from Dapa or γ-Glu. Most preferably, S_{A} is γ-Glu.

### The Group S_{B}

S_{B} may be a linker of general formula:

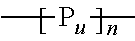

wherein P_{U} is a polymeric unit and n is 1, 2, 3, 4, 5, 6, 7, 8, 9 or 10. One terminus of the linker S_{B} is an —NH, —NR, —S or —O, wherein R may be alkyl, a protecting group or may form a linkage to another part of the polymeric unit; while the other is a bond or CO—, SO— or SO₂—. Accordingly, each polymeric unit P_{U} may be bound at each side by amide, sulfinamide, sulfonamide, or ester linkages or by amino, ether, or thioether linkages depending upon the nature of Y and X and the corresponding linking groups on Z¹, S_{A}, and Lys.

In some embodiments, each P_{U} may be independently a unit of formula:

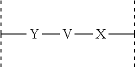

wherein:
Y may be —NH, —NR, —S or —O, wherein R may be alkyl, a protecting group or may form a linkage to another part of the spacer, with the remaining valency forming a linkage to Z¹;
X may be a bond, CO—, SO—, or SO₂—, with the remaining valency forming a linkage to the ψ side chain;
and V is a bivalent organic moiety linking Y and X.

In some embodiments, V is the α-carbon of a natural or unnatural amino acid, that is V is —CHR^{AA}—, wherein R^{AA} is an amino acid side chain; or V is an optionally substituted C₁₋₆alkylene, or V is a chain comprising one or more units of ethylene glycol in series, also known as PEG chain, for example, —CH₂CH₂—(OCH₂CH₂)ₘ—O—(CH₂)ₚ—, where m is 0, 1, 2, 3, 4, or 5, and p is 1, 2, 3, 4, or 5; when X is CO—, p is preferably 1, 3, 4, or 5. Optional alkylene substituents include fluoro, methyl, hydroxy, hydroxymethyl, and amino.

Preferred P_{U} units include:
(i). Single amino acid residues: P_{U}ⁱ;
(ii). Dipeptide residues: P_{U}ⁱⁱ; and
(iii). Amino-(PEG)ₘ-carboxylic acid residues: P_{U}ⁱⁱ,
and may be present in any combination or order. For example, S_{B} may comprise one or more of each of P_{U}ⁱ, P_{U}ⁱⁱ, and P_{U}ⁱⁱⁱ in any order, or may comprise one or more units of P_{U}ⁱ, P_{U}ⁱⁱ, and P_{U}ⁱⁱⁱ only, or one of more units selected from P_{U}ⁱ and P_{U}ⁱⁱ, P_{U}ⁱ and P_{U}ⁱⁱⁱ, or P_{U}ⁱⁱ and P_{U}ⁱⁱⁱ.
   (i). P_{U}ⁱ Single Amino Acid Residues

Each P_{U}ⁱ may be independently selected from any natural or unnatural amino acid residue and, for example, may be selected from Gly, Pro, Ala, Val, Leu, Ile, Met, Cys, Phe, Tyr, Trp, His, Lys, Arg, Gln, Asn, α-Glu, γ-Glu, Asp, Ser Thr, Dapa, Gaba, Aib, β-Ala, 5-aminopentanoyl, 6-aminohexanoyl, 7-aminoheptanoyl, 8-aminooctanoyl, 9-aminononanoyl, and 10-aminodecanoyl. Preferably, Pul amino acid residues are selected from Gly, Ser, Ala, Thr, and Cys, more preferably from Gly and Ser.

In some embodiments, S_{B} is —(P_{U}ⁱ)ₙ—, wherein n is 1 to 8, more preferably 5 to 7, most preferably 6. In some preferred embodiments, S_{B} is —(P_{U}ⁱ)ₙ—, n is 6 and each P_{U}ⁱ is independently selected from Gly or Ser, with a preferred sequence being-Gly-Ser-Gly-Ser-Gly-Gly-.

(ii). P_{U}ⁱⁱ Dipeptide Residues

Each P_{U}ⁱⁱ may be independently selected from any dipeptide residue comprising two natural or unnatural amino acid residues bound by an amide linkage. Preferred P_{U}ⁱⁱ dipeptide residues include Gly-Gly, Gly-Ser, Ser-Gly, Gly-Ala, Ala-Gly, and Ala-Ala, more preferably Gly-Ser and Gly-Gly.

In some embodiments, S_{B} is —(P_{U}ⁱ)ₙ—, wherein n is 2 to 4, more preferably 3, and each P_{U}ⁱⁱ is independently selected from Gly-Ser and Gly-Gly. In some preferred embodiments S_{B} is —(P_{U}ⁱⁱ)ₙ—, n is 3 and each P_{U}ⁱⁱ is independently selected from Gly-Ser and Gly-Gly, with a preferred sequence being -(Gly-Ser)-(Gly-Ser)-(Gly-Gly) (SEQ ID NO: 85).

Amino acids having stereogenic centres within P_{U}ⁱ and Puⁱⁱ may be racemic, enantioenriched, or enantiopure. In some embodiments, the or each amino acid is independently an L-amino acid. In some embodiments, the or each amino acid is independently a D-amino acid.

(iii). P_{U}ⁱⁱⁱ Amino-(PEG)ₘ-Carboxylic Acid Residues

Each P_{U}ⁱⁱⁱ may be independently a residue of general formula:

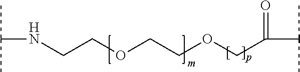

wherein m is 0, 1, 2, 3, 4, or 5, preferably 1 or 2, and p is 1, 3, 4, or 5, preferably 1.

In some embodiments, m is 1 and p is 1, that is, Pull is a residue of 8-amino-3,6-dioxaoctanoic acid (also known as {2-[2-aminoethoxy]ethoxy}acetic acid and H₂N-PEG₃-COOH). This residue is referred to herein as -PEG₃-.

Other, longer, PEG chains are also known in the art. For example, 11-amino-3,6,9-trioxaundecanoic acid (also known as H₂N-PEG₄-COOH) or -PEG₄-).

In some embodiments, S_{B} is —(P_{U}ⁱⁱⁱ)ₙ—, wherein n is 1 to 3, more preferably 2.

Most preferably, S_{B} is-PEG₃-PEG₃-.

### Preferred Combinations

It will be understood that the above preferences may be independently combined to give preferred —Z¹ and —Z²-Z¹ moieties.

Some preferred —Z¹ and —Z²-Z¹ moieties are shown below (in each case, --- indicates the point of attachment to the side chain of the amino acid component of ψ:
(i) [17-carboxy-heptadecanoyl]-isoGlu-Peg3-Peg3

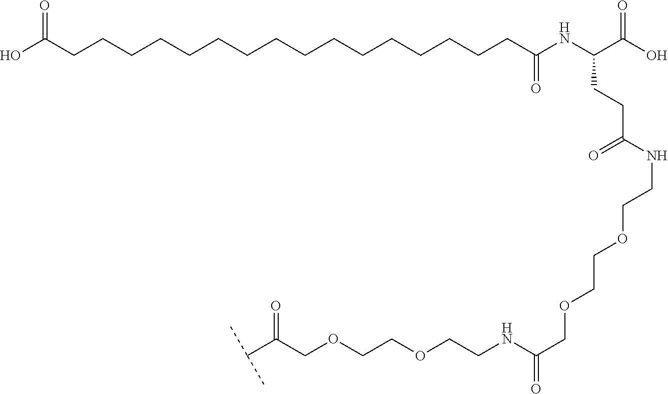

(ii) [17-carboxy-heptadecanoyl]-isoGlu

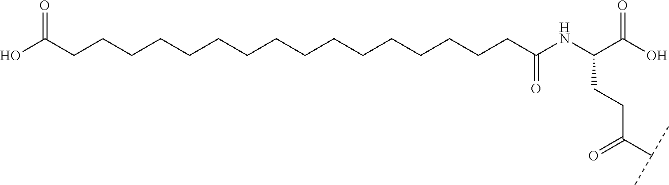

(iii) Octadecanoyl-isoGlu-Peg3-Peg3

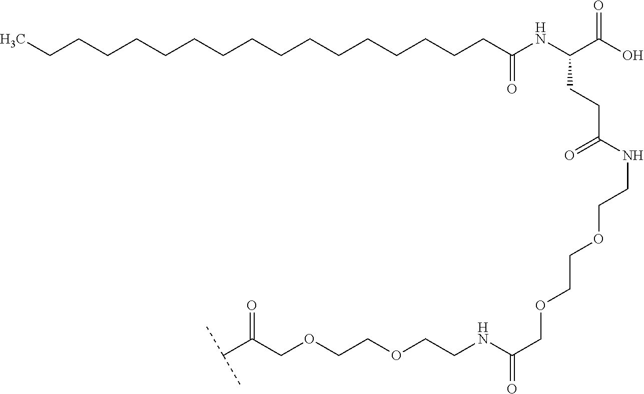

(iv) Eicosanoyl-isoGlu-Peg3-Peg3

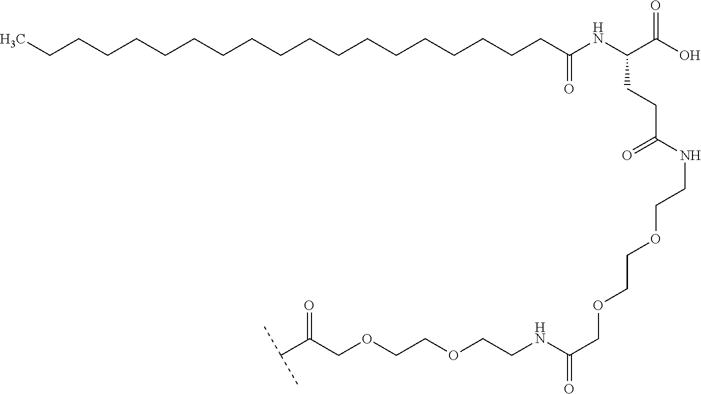

(v) [19-carboxy-nonadecanoyl]-isoGlu-Peg3-Peg3

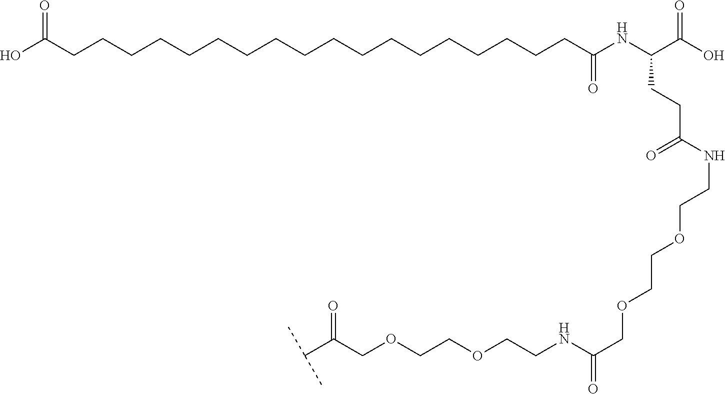

(vi) Octadecanoyl-Dapa-Peg3-Peg3

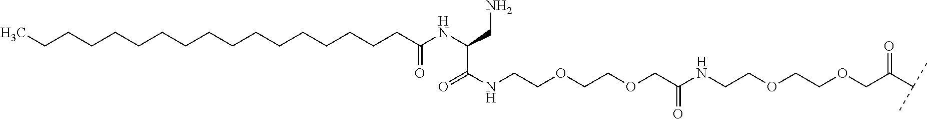

(vii) Hexadecanoyl-isoGlu

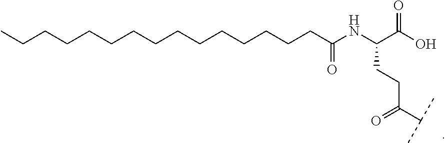

(viii) (19-Carboxy-nonadecanoyl)-[(Piperazine-1-yl)-acetyl]-Peg3-Peg3

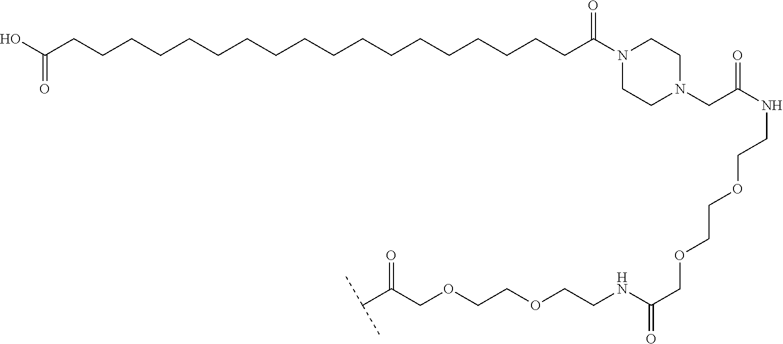

In particular embodiments, the GlP analogue used in the methods of the invention is selected from those listed in Table 1.

TABLE 1

| Compound | |
|---|---|
| No. | Sequence |
| 1 | H-Y-Aib-EGTFISDYSIELDK-K(Hexadecanoyl-isoGlu)- |
| | HQQDFVNWLLAQGPSSGAPPPS-NH₂ (SEQ ID NO: 1) |
| 2 | H-Y-Aib-EGTFISDYSIELD-K(Hexadecanoyl-isoGlu)- |
| | IHQQDFVNWLLAQGPSSGAPPPS-NH₂ (SEQ ID NO: 2) |
| 3 | H-Y-Aib-EGTFISDYSIELEK-K(Hexadecanoyl-isoGlu)- |
| | HQQDFVNWLLAQGPSSGAPPPS-NH₂ (SEQ ID NO: 3) |
| 4 | H-Y-Aib-EGTFISDYSIELDKIHQQDFVNWLLAQGPSSGAPPPS- |
| | K([19-carboxy-nonadecanoyl]-isoGlu-Peg3-Peg3)-NH₂ (SEQ ID |
| | NO: 4) |
| 5 | H-Y-Aib-EGTFISDYSIELDKIHQQDFVNWLLAQGPSSGAPPPS- |
| | K(Hexadecanoyl-isoGlu)-NH₂ (SEQ ID NO: 5) |
| 6 | H-Y-Aib-EGTFISDYSIELDKIHQQDFVNWLLAQ-K(Hexadecanoyl- |
| | isoGlu)-NH₂ (SEQ ID NO: 6) |
| 7 | H-Y-Aib-EGTFISDYSIELDKIHQQDFVNWLLAQKG- |
| | K(Hexadecanoyl-isoGlu)-NH₂ (SEQ ID NO: 7) |
| 8 | H-Y-Aib-EGTFISDYSIELDK-K([19-carboxy-nonadecanoyl]-isoGlu- |
| | Peg3-Peg3)-HQQDFVNYLLAQGPSSGAPPPS-NH₂ (SEQ ID NO: |
| | 8) |
| 9 | H-Y-Aib-EGTFISDYSIELDK-K([19-carboxy-nonadecanoyl]-isoGlu- |
| | Peg3-Peg3)-HQQDFVNWLLAQGPSSGAPPPS-NH₂ (SEQ ID NO: |
| | 9) |
| 10 | H-Y-Aib-EGTFISDYSIELDK-K([19-carboxy-nonadecanoyl]-isoGlu- |
| | Peg3-Peg3)-AAQDFVNWLLAQGPSSGAPPPS-NH₂ (SEQ ID NO: |
| | 10) |
| 11 | H-Y-Aib-EGTFISDYSIELEK-K([19-carboxy-nonadecanoyl]-isoGlu- |
| | Peg3-Peg3)-AAKEFVNWLLAQGPSSGAPPPS-NH₂ (SEQ ID NO: |
| | 11) |
| 12 | H-Y-Aib-EGTFISDYSIELEK-K([19-carboxy-nonadecanoyl]-isoGlu- |
| | Peg3-Peg3)-AQRAFVEWLLAQGPSSGAPPPS-NH₂ (SEQ ID NO: |
| | 12) |
| 13 | H-Y-Aib-EGTFISDYSIELEKIAQRAFVEWLLAQGPSSGAPPPS- |
| | K([19-carboxy-nonadecanoyl]-isoGlu-Peg3-Peg3)-NH₂ (SEQ ID |
| | NO: 13) |
| 14 | H-Y-Aib-EGTFISDYSIELEKIAQRAFVEWLLAQ-K([19-carboxy- |
| | nonadecanoyl]-isoGlu-Peg3-Peg3)-NH₂ (SEQ ID NO: 14) |
| 15 | H-Y-Aib-EGTFISDYSIELDK-K([19-carboxy-nonadecanoyl]-isoGlu- |
| | Peg3-Peg3)-AAQDFVNWLLAGPSSGAPPPS-NH₂ (SEQ ID NO: |
| | 15) |
| 16 | H-Y-Aib-EGTFISDYSIELDKIAAQDFVNWLLAGPSSGAPPPS- |
| | K([19-carboxy-nonadecanoyl]-isoGlu-Peg3-Peg3)-NH₂ (SEQ ID |
| | NO: 16) |
| 17 | H-Y-Aib-EGTFISDYSIELDK-K([19-Carboxy-nonadecanoyl]-isoGlu- |
| | Peg3-Peg3)-AQRAFVEWLLAQGPSSGAPPPS-NH₂ (SEQ ID NO: |
| | 17) |
| 18 | H-Y-Aib-EGTFISDYSIELDK-K([19-Carboxy-nonadecanoyl]-isoGlu- |
| | Peg3-Peg3)-AQRAFIEWLLAQGPSSGAPPPS-NH₂ (SEQ ID NO: |
| | 18) |
| 19 | H-Y-Aib-EGTFISDYSIELDK-K((19-Carboxy-nonadecanoyl)- |
| | [(Piperazine-1-yl)-acetyl]-Peg3-Peg3)- |
| | AQRAFIEWLLAQGPSSGAPPPS-NH₂ (SEQ ID NO: 19) |
| 20 | H-Y-Aib-EGTFISDYSIELDK-K((19-Carboxy-nonadecanoyl)- |
| | [(Piperazine-1-yl)-acetyl]-Peg3-Peg3)- |
| | AQRAFVEWLLAQGPSSGAPPPS-NH₂ (SEQ ID NO: 20) |
| 21 | H-Y-Aib-EGTFISDYSIELDK-K((19-Carboxy-nonadecanoyl)- |
| | [(Piperazine-1-yl)-acetyl]-Peg3-Peg3)- |
| | AQKEFVEWLLAAGPSSGAPPPS-NH₂ (SEQ ID NO: 21) |
| 22 | H-Y-Aib-EGTFISDYSIELDK-K([19-Carboxy-nonadecanoyl]-isoGlu- |
| | Peg3-Peg3)-AQKEFVEWLLAAGPSSGAPPPS-NH₂ (SEQ ID NO: |
| | 22) |
| 23 | H-Y-Aib-EGTFISDYSIELDKIAQRAFIEWLLAGPSSGAPPPS-K([19- |
| | Carboxy-nonadecanoyl]-isoGlu-Peg3-Peg3)-NH₂ (SEQ ID NO: 23) |
| 24 | H-Y-Aib-EGTFISDYSIELDKIAQKEFIEWLLAGPSSGAPPPS-K([19- |
| | Carboxy-nonadecanoyl]-isoGlu-Peg3-Peg3)-NH₂ (SEQ ID NO: 24) |
| 25 | H-Y-Aib-EGTFISDYSIELDKIAAQDFIEWLLAGPSSGAPPPS-K([19- |
| | Carboxy-nonadecanoyl]-isoGlu-Peg3-Peg3)-NH₂ (SEQ ID NO: 25) |
| 26 | H-Y-Aib-EGTFISDYSIELDKIAAQDFIEWLLAGPSSGAPPPS-K((19- |
| | Carboxy-nonadecanoyl)-[(Piperazine-1-yl)-acetyl]-Peg3-Peg3)-NH₂ |
| | (SEQ ID NO: 26) |
| 27 | H-Y-Aib-EGTFISDYSIELDKIAAQDFVEWLLAGPSSGAPPPS- |
| | K([19-Carboxy-nonadecanoyl]-isoGlu-Peg3-Peg3)-NH₂ (SEQ ID |
| | NO: 27) |
| 28 | H-Y-Aib-EGTFISDYSIELDKIAQRAFIEWLLAQGPSSGAPPPS- |
| | K([19-Carboxy-nonadecanoyl]-isoGlu-Peg3-Peg3)-NH₂ (SEQ ID |
| | NO: 28) |
| 29 | H-Y-Aib-EGTFISDYSIELDK-K([19-Carboxy-nonadecanoyl]-isoGlu- |
| | Peg3-Peg3)-AAQAFVNWLLAGPSSGAPPPS-NH₂ (SEQ ID NO: |
| | 29) |
| 30 | H-Y-Aib-EGTFISDYSIELDK-K([19-Carboxy-nonadecanoyl]-isoGlu- |
| | Peg3-Peg3)-AAQDFVNWLLAAGPSSGAPPPS-NH₂ (SEQ ID NO: |
| | 30) |
| 31 | H-Y-Aib-EGTFISDYSIELDK-K([19-Carboxy-nonadecanoyl]-isoGlu- |
| | Peg3-Peg3)-AAQDFINWLLAGPSSGAPPPS-NH₂ (SEQ ID NO: 31) |
| 32 | H-Y-Aib-EGTFISDYSIELDK-K([19-Carboxy-nonadecanoyl]-isoGlu- |
| | Peg3-Peg3)-AAQDFIEWLLAGPSSGAPPPS-NH₂ (SEQ ID NO: 32) |
| 33 | H-Y-Aib-EGTFISDYSIELDK-K((19-Carboxy-nonadecanoyl)- |
| | [(Piperazine-1-yl)-acetyl]-Peg3-Peg3)- |
| | AAQDFIEWLLAGPSSGAPPPS-NH₂ (SEQ ID NO: 33) |
| 34 | H-Y-Aib-EGTFISDYSIELD-K([19-Carboxy-nonadecanoyl]-isoGlu- |
| | Peg3-Peg3)-IAQRAFIEWLLAQGPSSGAPPPS-NH₂ (SEQ ID NO: |
| | 34) |
| 35 | H-Y-Aib-EGTFISDYS-K([19-Carboxy-nonadecanoyl]-isoGlu-Peg3- |
| | Peg3)-ELDKIAQRAFIEWLLAQGPSSGAPPPS-NH₂ (SEQ ID NO: |
| | 35) |
| 36 | H-Y-DAla-EGTFISDYSIELDK-K([19-Carboxy-nonadecanoyl]- |
| | isoGlu-Peg3-Peg3)-AQRAFIEWLLAQGPSSGAPPPS-NH₂ (SEQ ID |
| | NO: 36) |
| 37 | H-Y-DAla-EGTFISDYSIELDKIAAQDFIEWLLAGPSSGAPPPS- |
| | K((19-Carboxy-nonadecanoyl)-[(Piperazine-1-yl)-acetyl]-Peg3- |
| | Peg3)-NH₂ (SEQ ID NO: 37) |
| 38 | H-Y-Aib-EGTFISDYSIELDK-K([19-Carboxy-nonadecanoyl]-isoGlu- |
| | Peg3-Peg3)-AAQDFIEWLLAQGPSSGAPPPS-NH₂ (SEQ ID NO: |
| | 38) |
| 39 | H-Y-Aib-EGTFISDYSIELDK-K([19-Carboxy-nonadecanoyl]-isoGlu- |
| | Peg3-Peg3)-AAQDFINWLLAQGPSSGAPPPS-NH₂ (SEQ ID NO: |
| | 39) |
| 40 | H-Y-Aib-EGTFISDYSIELDK-K([19-Carboxy-nonadecanoyl]-isoGlu- |
| | Peg3-Peg3)-AAQAFIEWLLAQGPSSGAPPPS-NH₂ (SEQ ID NO: |
| | 40) |
| 41 | H-Y-Aib-EGTFISDYSIELDK-K((19-Carboxy-nonadecanoyl)- |
| | [(Piperazine-1-yl)-acetyl]-Peg3-Peg3)- |
| | AAQAFIEWLLAQGPSSGAPPPS-NH₂ (SEQ ID NO: 41) |

e. Clinical Utility

The methods of the invention can be particular effective in reducing body weight, maintaining weight loss, and treating obesity (e.g., by control of appetite, feeding, food intake, calorie intake, and/or energy expenditure and lipoiysis), including morbid obesity, as well as associated diseases and health conditions including but not limited to obesity linked conditions and symptoms.

f. Pharmaceutical Compositions

The GLP-1 agonists and GlP analogues (e.g., GlP agonist compounds) used in the methods of the present invention may be formulated separately as pharmaceutical compositions (and administered separately), each including a therapeutically effective amount of a compound employed in the context of the invention, or a salt thereof, in a pharmaceutically acceptable carrier. In some embodiments, the pharmaceutical composition is formulated as a liquid suitable for administration by injection or infusion, or which is formulated to cause slow release of the GlP analogue.

The therapeutically effective amount of each compound used in the method of the present invention will depend, e.g., on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person. The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. Suitable pH buffering agents may be, e.g., phosphate, citrate, acetate, lactate, maleate, tris/hydroxymethyl)aminomethane (TRIS), N-Tris(hydroxymethyl)methyl-3-aminopropanesulphonic acid (TAPS), ammonium bicarbonate, diethanolamine, histidine, which in certain embodiments is a preferred buffer, arginine, lysine, or acetate or mixtures thereof. The term further encompasses any agents listed in the US Pharmacopeia for use in animals, including humans.

g. Synthesis of Compounds Used in the Methods of the Invention

The skilled person will be well aware of suitable techniques for preparing the compounds employed in the context of the invention. For examples of suitable chemistry, see, e.g., WO98/08871, WO2016/066744, WO00/55184, WO00/55119, Madsen et al. (J. Med. Chem. 2007, 50, 6126-32), and Knudsen et al. 2000 (J. Med Chem. 43, 1664-1669).

A nucleic acid molecule may encode the amino acid sequence of any of Formula I to Ill or a precursor thereof. The amino acid sequence encoded can be regarded as a precursor of a compound of the invention.

Typically, such nucleic acid sequences will be provided as expression constructs wherein the encoding nucleic acid is in functional linkage with appropriate control sequences to direct its expression. The expression construct may be provided in the context of a host cell capable of expressing (and optionally also secreting) the amino acid precursor, or in a cell-free expression system.

The compounds used in the methods of the invention may also be manufactured by standard peptide synthetic methods, e.g. by standard solid-phase or liquid-phase methodology, either stepwise or by fragment assembly, and isolating and purifying the final peptide compound product, or by any combinations of recombinant and synthetic methods.

It may be preferable to synthesize the peptide compounds of the invention by means of solid-phase or liquid-phase peptide synthesis. In this context, reference may be made to WO 98/11125 or, inter alia, Fields, G. B. et al., “Principles and Practice of Solid-Phase Peptide Synthesis”; in: Synthetic Peptides, Gregory A. Grant (ed.), Oxford University Press (2nd edition, 2002) and the synthesis examples herein.

h. Sequence listing

Sequence identifier numbers (SEQ ID NOs) are assigned to sequences herein as follows:
SEQ ID NOs: 1-41 & 118-GlP analogue sequences
SEQ ID NOs: 42, 48 & 50-GlP analogue Formulas I, II & III respectively
SEQ ID NOs: 43-47-Alternative sequences within GlP analogue Formulas I, II & III
SEQ ID NO: 49 & 86-88-skipped
SEQ ID NOs: 51-84 & 96-GlP analogue sequences wherein the substituent may be any substituent herein
SEQ ID NO: 85-Linker sequence
SEQ ID NOs: 89-95-Sequences of GLP-1 analogues exenatide, liraglutide, semaglutide, dulaglutide, lixisenatide, albiglutide and taspoglutide respectively
SEQ ID NOs: 97-102 & 115-Embodiments of GlP analogue Formula I
SEQ ID NOs: 103-108 & 116-Embodiments of GlP analogue Formula II
SEQ ID NOs: 109-114 & 117-Embodiments of GlP analogue Formula III

### NUMBERED PARAGRAPHS

The present invention will now be described by way of numbered paragraphs.
1. A method of increasing weight loss in a subject in need thereof, the method comprising:
   (i) providing a subject undergoing treatment with a GLP-1 agonist for a period of time during which weight loss in the subject plateaus and/or the subject is or becomes GLP-1 treatment resistant, and
   (ii) following step (i), administering to the subject (a) a GLP-1 agonist and (b) a GlP agonist, each in an amount that together is sufficient to increase weight loss in the subject.
2. A method of increasing weight loss in a subject in need thereof, the method comprising:
   (i) providing a subject who fails to respond to treatment with a GLP-1 agonist, and
   (ii) administering to the subject (a) the GLP-1 agonist and (b) a GlP agonist, each in an amount that together is sufficient to increase weight loss in the subject.
3. A method of increasing weight loss in a subject in need thereof, the method comprising:
   (i) providing a subject undergoing treatment with a GLP-1 agonist for a period of at least 1 month, and
   (ii) following step (i), administering to the subject (a) a GLP-1 agonist and (b) a GlP agonist, each in an amount that together is sufficient to increase weight loss in the subject.
4. A method of maintaining weight loss in a subject in need thereof, the method comprising:
   (i) providing a subject undergoing treatment with a GLP-1 agonist, wherein the subject has experienced weight loss, and
   (ii) following step (i), administering to the subject (a) a GLP-1 agonist and (b) a GlP agonist, each in an amount that together is sufficient to maintain the weight loss in the subject.
5. A method of increasing weight loss in a subject in need thereof, the method comprising:
   (i) providing a subject undergoing treatment with a GLP-1 agonist for a period of time during which weight loss in the subject plateaus and/or the subject is or becomes GLP-1 treatment resistant and there is a need to achieve additional weight loss in the subject, and
   (ii) following step (i), administering to the subject (a) a GLP-1 agonist and (b) a GlP agonist, each in an amount that together is sufficient to achieve the additional weight loss in the subject.
6. A method of achieving additional weight loss in a subject in need thereof, the method comprising:
   (i) providing a subject undergoing treatment with a GLP-1 agonist for a period of time during which weight loss is achieved and there is a need to achieve additional weight loss in the subject, and
   (ii) following step (i), administering to the subject (a) a GLP-1 agonist and (b) a GlP agonist, each in an amount that together is sufficient to achieve the additional weight loss in the subject.
7. A combination of a GLP-1 agonist and a GlP agonist for use in achieving weight loss in a subject, wherein weight loss in the subject is achieved by use of the GLP-1 agonist and the GlP agonist in a method, wherein the method is for increasing weight loss in a subject in need thereof, the method comprising:
   (i) providing a subject undergoing treatment with a GLP-1 agonist for a period of time during which weight loss in the subject plateaus and/or the subject is or becomes GLP-1 treatment resistant, and
   (ii) following step (i), administering to the subject (a) a GLP-1 agonist and (b) a GlP agonist, each in an amount that together is sufficient to increase weight loss in the subject.
8. A combination of a GLP-1 agonist and a GlP agonist for use in achieving weight loss in a subject, wherein weight loss in the subject is achieved by use of the GLP-1 agonist and the GlP agonist in a method, wherein the method is for increasing weight loss in a subject in need thereof, the method comprising:
   (i) providing a subject who fails to respond to treatment with a GLP-1 agonist, and
   (ii) administering to the subject (a) the GLP-1 agonist and (b) a GlP agonist, each in an amount that together is sufficient to increase weight loss in the subject.
9. A combination of a GLP-1 agonist and a GlP agonist for use in achieving weight loss in a subject, wherein weight loss in the subject is achieved by use of the GLP-1 agonist and the GlP agonist in a method, wherein the method is for increasing weight loss in a subject in need thereof, the method comprising:
   (i) providing a subject undergoing treatment with a GLP-1 agonist for a period of at least 1 month, and
   (ii) following step (i), administering to the subject (a) a GLP-1 agonist and (b) a GlP agonist, each in an amount that together is sufficient to increase weight loss in the subject.
10. A combination of a GLP-1 agonist and a GlP agonist for use in achieving weight loss in a subject, wherein weight loss in the subject is achieved by use of the GLP-1 agonist and the GlP agonist in a method, wherein the method is for maintaining weight loss in a subject in need thereof, the method comprising:
   (i) providing a subject undergoing treatment with a GLP-1 agonist, wherein the subject has experienced weight loss, and
   (ii) following step (i), administering to the subject (a) a GLP-1 agonist and (b) a GlP agonist, each in an amount that together is sufficient to maintain the weight loss in the subject.
11. A combination of a GLP-1 agonist and a GlP agonist for use in achieving weight loss in a subject, wherein weight loss in the subject is achieved by use of the GLP-1 agonist and the GlP agonist in a method, wherein the method is for increasing weight loss in a subject in need thereof, the method comprising:
   (i) providing a subject undergoing treatment with a GLP-1 agonist for a period of time during which weight loss in the subject plateaus and/or the subject is or becomes GLP-1 treatment resistant and there is a need to achieve additional weight loss in the subject, and
   (ii) following step (i), administering to the subject (a) a GLP-1 agonist and (b) a GlP agonist, each in an amount that together is sufficient to achieve the additional weight loss in the subject.
12. A combination of a GLP-1 agonist and a GlP agonist for use in achieving weight loss in a subject, wherein weight loss in the subject is achieved by use of the GLP-1 agonist and the GlP agonist in a method, wherein the method is for achieving additional weight loss in a subject in need thereof, the method comprising:
   (i) providing a subject undergoing treatment with a GLP-1 agonist for a period of time during which weight loss is achieved and there is a need to achieve additional weight loss in the subject, and
   (ii) following step (i), administering to the subject (a) a GLP-1 agonist and (b) a GlP agonist, each in an amount that together is sufficient to achieve the additional weight loss in the subject.
13. Use of a combination of a GLP-1 agonist and a GlP agonist for manufacture of a medicament for use in a method of achieving weight loss in a subject, wherein the method is for increasing weight loss in a subject in need thereof, the method comprising:
   (i) providing a subject undergoing treatment with a GLP-1 agonist for a period of time during which weight loss in the subject plateaus and/or the subject is or becomes GLP-1 treatment resistant, and
   (ii) following step (i), administering to the subject (a) a GLP-1 agonist and (b) a GlP agonist, each in an amount that together is sufficient to increase weight loss in the subject.
14. Use of a combination of a GLP-1 agonist and a GlP agonist for manufacture of a medicament for use in a method of achieving weight loss in a subject, wherein the method is for increasing weight loss in a subject in need thereof, the method comprising:
   (i) providing a subject who fails to respond to treatment with a GLP-1 agonist, and
   (ii) administering to the subject (a) the GLP-1 agonist and (b) a GlP agonist, each in an amount that together is sufficient to increase weight loss in the subject.
15. Use of a combination of a GLP-1 agonist and a GlP agonist for manufacture of a medicament for use in a method of achieving weight loss in a subject, wherein the method is for increasing weight loss in a subject in need thereof, the method comprising:
   (i) providing a subject undergoing treatment with a GLP-1 agonist for a period of at least 1 month, and
   (ii) following step (i), administering to the subject (a) a GLP-1 agonist and (b) a GlP agonist, each in an amount that together is sufficient to increase weight loss in the subject.
16. Use of a combination of a GLP-1 agonist and a GlP agonist for manufacture of a medicament for use in a method of achieving weight loss in a subject, wherein the method is for maintaining weight loss in a subject in need thereof, the method comprising:
   (i) providing a subject undergoing treatment with a GLP-1 agonist, wherein the subject has experienced weight loss, and
   (ii) following step (i), administering to the subject (a) a GLP-1 agonist and (b) a GlP agonist, each in an amount that together is sufficient to maintain the weight loss in the subject.
17. Use of a combination of a GLP-1 agonist and a GlP agonist for manufacture of a medicament for use in a method of achieving weight loss in a subject, wherein the method is for increasing weight loss in a subject in need thereof, the method comprising:
   (i) providing a subject undergoing treatment with a GLP-1 agonist for a period of time during which weight loss in the subject plateaus and/or the subject is or becomes GLP-1 treatment resistant and there is a need to achieve additional weight loss in the subject, and
   (ii) following step (i), administering to the subject (a) a GLP-1 agonist and (b) a GlP agonist, each in an amount that together is sufficient to achieve the additional weight loss in the subject.
18. Use of a combination of a GLP-1 agonist and a GlP agonist for manufacture of a medicament for use in a method of achieving weight loss in a subject, wherein the method is for achieving additional weight loss in a subject in need thereof, the method comprising:
   (i) providing a subject undergoing treatment with a GLP-1 agonist for a period of time during which weight loss is achieved and there is a need to achieve additional weight loss in the subject, and
   (ii) following step (i), administering to the subject (a) a GLP-1 agonist and (b) a GlP agonist, each in an amount that together is sufficient to achieve the additional weight loss in the subject.
19. The method or the combination for use of any one of paragraphs 1-18, wherein the subject has a BMI of >30 kg/m², such as an initial BMI of >30 kg/m² before the method is used on the subject.
20. The method, the combination for use, or the use of any one of paragraphs 1-19, wherein the GLP-1 agonist in step (ii) is the same as the GLP-1 agonist in step (i).
21. The method, the combination for use, or the use of any one of paragraphs 1-20, wherein the GLP-1 agonist is selected from exenatide, liraglutide, semaglutide, dulaglutide, lixisenatide, albiglutide, and taspoglutide.
22. The method, the combination for use, or the use of paragraph 21, wherein the GLP-1 agonist is semaglutide.
23. The method, the combination for use, or the use of any one of paragraphs 1-22, wherein the GlP agonist is any GlP agonist described herein.
24. The method, the combination for use, or the use of any one of paragraphs 1-23, wherein the GlP agonist is selected from the following, and pharmaceutically acceptable salts thereof:

(SEQ ID NO: 1)
H-Y-Aib-EGTFISDYSIELDK-K(Hexadecanoyl-isoGlu)-
HQQDFVNWLLAQGPSSGAPPPS-NH₂;
(Compound 1)
(SEQ ID NO: 2)
H-Y-Aib-EGTFISDYSIELD-K(Hexadecanoyl-isoGlu)-
IHQQDFVNWLLAQGPSSGAPPPS-NH₂;
(Compound 2)
(SEQ ID NO: 3)
H-Y-Aib-EGTFISDYSIELEK-K(Hexadecanoyl-isoGlu)-
HQQDFVNWLLAQGPSSGAPPPS-NH₂;
(Compound 3)
(SEQ ID NO: 4)
H-Y-Aib-EGTFISDYSIELDKIHQQDFVNWLLAQGPSSGAPPPS-K([19-carboxy-
nonadecanoyl]-isoGlu-Peg3-Peg3)-NH₂;
(Compound 4)
(SEQ ID NO: 5)
H-Y-Aib-EGTFISDYSIELDKIHQQDFVNWLLAQGPSSGAPPPS-K(Hexadecanoyl-
isoGlu)-NH₂;
(Compound 5)
(SEQ ID NO: 6)
H-Y-Aib-EGTFISDYSIELDKIHQQDFVNWLLAQ-K(Hexadecanoyl-isoGlu)-NH₂;
(Compound 6)
(SEQ ID NO: 7)
H-Y-Aib-EGTFISDYSIELDKIHQQDFVNWLLAQKG-K(Hexadecanoyl-isoGlu)-NH₂;
(Compound 7)
(SEQ ID NO: 8)
H-Y-Aib-EGTFISDYSIELDK-K([19-carboxy-nonadecanoyl]-isoGlu-Peg3-Peg3)-
HQQDFVNYLLAQGPSSGAPPPS-NH₂;
(Compound 8)
(SEQ ID NO: 9)
H-Y-Aib-EGTFISDYSIELDK-K([19-carboxy-nonadecanoyl]-isoGlu-Peg3-Peg3)-
HQQDFVNWLLAQGPSSGAPPPS-NH₂;
(Compound 9)
(SEQ ID NO: 10)
H-Y-Aib-EGTFISDYSIELDK-K([19-carboxy-nonadecanoyl]-isoGlu-Peg3-Peg3)-
AAQDFVNWLLAQGPSSGAPPPS-NH₂;
(Compound 10)
(SEQ ID NO: 11)
H-Y-Aib-EGTFISDYSIELEK-K([19-carboxy-nonadecanoyl]-isoGlu-Peg3-Peg3)-
AAKEFVNWLLAQGPSSGAPPPS-NH₂;
(Compound 11)
(SEQ ID NO: 12)
H-Y-Aib-EGTFISDYSIELEK-K([19-carboxy-nonadecanoyl]-isoGlu-Peg3-Peg3)-
AQRAFVEWLLAQGPSSGAPPPS-NH₂;
(Compound 12)
(SEQ ID NO: 13)
H-Y-Aib-EGTFISDYSIELEKIAQRAFVEWLLAQGPSSGAPPPS-K([19-carboxy-
nonadecanoyl]-isoGlu-Peg3-Peg3)-NH₂;
(Compound 13)
(SEQ ID NO: 14)
H-Y-Aib-EGTFISDYSIELEKIAQRAFVEWLLAQ-K([19-carboxy-nonadecanoyl]-
isoGlu-Peg3-Peg3)-NH₂;
(Compound 14)
(SEQ ID NO: 15)
H-Y-Aib-EGTFISDYSIELDK-K([19-carboxy-nonadecanoyl]-isoGlu-Peg3-Peg3)-
AAQDFVNWLLAGPSSGAPPPS-NH₂;
(Compound 15)
(SEQ ID NO: 16)
H-Y-Aib-EGTFISDYSIELDKIAAQDFVNWLLAGPSSGAPPPS-K([19-carboxy-
nonadecanoyl]-isoGlu-Peg3-Peg3)-NH₂;
(Compound 16)
(SEQ ID NO: 17)
H-Y-Aib-EGTFISDYSIELDK-K([19-Carboxy-nonadecanoyl]-isoGlu-Peg3-Peg3)-
AQRAFVEWLLAQGPSSGAPPPS-NH₂;
(Compound 17)
(SEQ ID NO: 18)
H-Y-Aib-EGTFISDYSIELDK-K([19-Carboxy-nonadecanoyl]-isoGlu-Peg3-Peg3)-
AQRAFIEWLLAQGPSSGAPPPS-NH₂;
(Compound 18)
(SEQ ID NO: 19)
H-Y-Aib-EGTFISDYSIELDK-K((19-Carboxy-nonadecanoyl)-[(Piperazine-1-yl)-acetyl]-
Peg3-Peg3)-AQRAFIEWLLAQGPSSGAPPPS-NH₂;
(Compound 19)
(SEQ ID NO: 20)
H-Y-Aib-EGTFISDYSIELDK-K((19-Carboxy-nonadecanoyl)-[(Piperazine-1-yl)-acetyl]-
Peg3-Peg3)-AQRAFVEWLLAQGPSSGAPPPS-NH₂;
(Compound 20)
(SEQ ID NO: 21)
H-Y-Aib-EGTFISDYSIELDK-K((19-Carboxy-nonadecanoyl)-[(Piperazine-1-yl)-acetyl]-
Peg3-Peg3)-AQKEFVEWLLAAGPSSGAPPPS-NH₂;
(Compound 21)
(SEQ ID NO: 22)
H-Y-Aib-EGTFISDYSIELDK-K([19-Carboxy-nonadecanoyl]-isoGlu-Peg3-Peg3)-
AQKEFVEWLLAAGPSSGAPPPS-NH₂;
(Compound 22)
(SEQ ID NO: 23)
H-Y-Aib-EGTFISDYSIELDKIAQRAFIEWLLAGPSSGAPPPS-K([19-Carboxy-
nonadecanoyl]-isoGlu-Peg3-Peg3)-NH₂;
(Compound 23)
(SEQ ID NO: 24)
H-Y-Aib-EGTFISDYSIELDKIAQKEFIEWLLAGPSSGAPPPS-K([19-Carboxy-
nonadecanoyl]-isoGlu-Peg3-Peg3)-NH₂;
(Compound 24)
(SEQ ID NO: 25)
H-Y-Aib-EGTFISDYSIELDKIAAQDFIEWLLAGPSSGAPPPS-K([19-Carboxy-
nonadecanoyl]-isoGlu-Peg3-Peg3)-NH₂;
(Compound 25)
(SEQ ID NO: 26)
H-Y-Aib-EGTFISDYSIELDKIAAQDFIEWLLAGPSSGAPPPS-K((19-Carboxy-
nonadecanoyl)-[(Piperazine-1-yl)-acetyl]-Peg3-Peg3)-NH₂;
(Compound 26)
(SEQ ID NO: 27)
H-Y-Aib-EGTFISDYSIELDKIAAQDFVEWLLAGPSSGAPPPS-K([19-Carboxy-
nonadecanoyl]-isoGlu-Peg3-Peg3)-NH₂;
(Compound 27)
(SEQ ID NO: 28)
H-Y-Aib-EGTFISDYSIELDKIAQRAFIEWLLAQGPSSGAPPPS-K([19-Carboxy-
nonadecanoyl]-isoGlu-Peg3-Peg3)-NH₂;
(Compound 28)
(SEQ ID NO: 29)
H-Y-Aib-EGTFISDYSIELDK-K([19-Carboxy-nonadecanoyl]-isoGlu-Peg3-Peg3)-
AAQAFVNWLLAGPSSGAPPPS-NH₂;
(Compound 29)
(SEQ ID NO: 30)
H-Y-Aib-EGTFISDYSIELDK-K([19-Carboxy-nonadecanoyl]-isoGlu-Peg3-Peg3)-
AAQDFVNWLLAAGPSSGAPPPS-NH₂;
(Compound 30)
(SEQ ID NO: 31)
H-Y-Aib-EGTFISDYSIELDK-K([19-Carboxy-nonadecanoyl]-isoGlu-Peg3-Peg3)-
AAQDFINWLLAGPSSGAPPPS-NH₂;
(Compound 31)
(SEQ ID NO: 32)
H-Y-Aib-EGTFISDYSIELDK-K([19-Carboxy-nonadecanoyl]-isoGlu-Peg3-Peg3)-
AAQDFIEWLLAGPSSGAPPPS-NH₂;
(Compound 32)
(SEQ ID NO: 33)
H-Y-Aib-EGTFISDYSIELDK-K((19-Carboxy-nonadecanoyl)-[(Piperazine-1-yl)-acetyl]-
Peg3-Peg3)-AAQDFIEWLLAGPSSGAPPPS-NH₂;
(Compound 33)
(SEQ ID NO: 34)
H-Y-Aib-EGTFISDYSIELD-K([19-Carboxy-nonadecanoyl]-isoGlu-Peg3-Peg3)-
IAQRAFIEWLLAQGPSSGAPPPS-NH₂;
(Compound 34)
(SEQ ID NO: 35)
H-Y-Aib-EGTFISDYS-K([19-Carboxy-nonadecanoyl]-isoGlu-Peg3-Peg3)-
ELDKIAQRAFIEWLLAQGPSSGAPPPS-NH₂;
(Compound 35)
(SEQ ID NO: 36)
H-Y-DAla-EGTFISDYSIELDK-K([19-Carboxy-nonadecanoyl]-isoGlu-Peg3-Peg3)-
AQRAFIEWLLAQGPSSGAPPPS-NH₂;
(Compound 36)
(SEQ ID NO: 37)
H-Y-DAla-EGTFISDYSIELDKIAAQDFIEWLLAGPSSGAPPPS-K((19-Carboxy-
nonadecanoyl)-[(Piperazine-1-yl)-acetyl]-Peg3-Peg3)-NH₂;
(Compound 37)
(SEQ ID NO: 38)
H-Y-Aib-EGTFISDYSIELDK-K([19-Carboxy-nonadecanoyl]-isoGlu-Peg3-Peg3)-
AAQDFIEWLLAQGPSSGAPPPS-NH₂;
(Compound 38)
(SEQ ID NO: 39)
H-Y-Aib-EGTFISDYSIELDK-K([19-Carboxy-nonadecanoyl]-isoGlu-Peg3-Peg3)-
AAQDFINWLLAQGPSSGAPPPS-NH₂;
(Compound 39)
(SEQ ID NO: 40)
H-Y-Aib-EGTFISDYSIELDK-K([19-Carboxy-nonadecanoyl]-isoGlu-Peg3-Peg3)-
AAQAFIEWLLAQGPSSGAPPPS-NH₂;
(Compound 40)
or
(SEQ ID NO: 41)
H-Y-Aib-EGTFISDYSIELDK-K((19-Carboxy-nonadecanoyl)-[(Piperazine-1-yl)-acetyl]-
Peg3-Peg3)-AAQAFIEWLLAQGPSSGAPPPS-NH₂.
(Compound 41)

25. The method, the combination for use, or the use of paragraph 24, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH2 (compound 41), or a pharmaceutically acceptable salt thereof.
26. The method, the combination for use, or the use of any one of paragraphs 1-25, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof; and wherein the GLP-1 agonist is selected from exenatide, liraglutide, semaglutide, dulaglutide, lixisenatide, albiglutide, and taspoglutide.
27. The method, the combination for use, or the use of any one of paragraphs 1-26, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof; and wherein the GLP-1 agonist is semaglutide.
28. The method, the combination for use, or the use of any one of paragraphs 1-27, wherein the GLP-1 agonist is administered by one or more of the following routes of administration: injection (including subcutaneous injection, intramuscular injection, intravenous injection, epidural injection and intraperitoneal injection), infusion pump (such as subcutaneous infusion pump), oral, sublingual, rectal, vaginal, parenteral, topical, dermal (such as by transdermal patch), nasal (such as by nasal spray) and pulmonary spray.
29. The method, the combination for use, or the use of paragraph 28, wherein the GLP-1 agonist is administered by subcutaneous injection.
30. The method, the combination for use, or the use of any one of paragraphs 1-29, wherein the GLP-1 agonist is administered at a dose of up to about 50 mg, such as up to about 40 mg, up to about 30 mg, up to about 20 mg or up to about 10 mg.
31. The method, the combination for use, or the use of any one of paragraphs 1-30, wherein the GLP-1 agonist is administered at a dose of from about 0.25 mg to about 50 mg, such as from about 0.25 mg to about 30 mg, from about 0.25 mg to about 20 mg, from about 0.25 mg to about 14 mg, from about 0.25 mg to about 10 mg, from about 1 mg to about 50 mg, such as from about 1 mg to about 30 mg, from about 1 mg to about 20 mg, from about 1 mg to about 14 mg, or from about 1 mg to about 10 mg.
32. The method, the combination for use, or the use of any one of paragraphs 1-31, wherein the GLP-1 agonist is administered at a dose of about 0.25 mg, about 0.5 mg, about 0.6 mg, about 1 mg, about 1.2 mg, about 1.5 mg, about 1.8 mg, about 2 mg, about 3 mg, about 4.5 mg, about 5 mg, about 7 mg, about 10 mg, about 14 mg, about 20 mg, about 30 mg or about 50 mg.
33. The method, the combination for use, or the use of any one of paragraphs 1-32, wherein the GLP-1 agonist is administered to the subject at least once, twice, or three times daily, or once every 2 days, every 3 days, every 4 days, every 5 days, every 6 days, every 7 days, every 8 days, every 9 days, every 10 days, every 11 days, every 12 days, every 13 days, every 14 days, every 28 days or every month.
34. The method, the combination for use, or the use of paragraph 33, wherein the GLP-1 agonist is administered to the subject once daily.
35. The method, the combination for use, or the use of paragraph 33, wherein the GLP-1 agonist is administered to the subject once weekly.
36. The method, the combination for use, or the use of any one of paragraphs 1-35, wherein in step (i) the GLP-1 agonist is administered to the subject as defined in any one of paragraphs 28-35.
37. The method, the combination for use, or the use of any one of paragraphs 1-36, wherein in step (ii) the GLP-1 agonist is administered to the subject as defined in any one of paragraphs 28-35.
38. The method, the combination for use, or the use of any one of paragraphs 1-37, wherein the GlP agonist is administered by one or more of the following routes of administration: injection (including subcutaneous injection, intramuscular injection, intravenous injection, epidural injection and intraperitoneal injection), infusion pump (such as subcutaneous infusion pump), oral, sublingual, rectal, vaginal, parenteral, topical, dermal (such as by transdermal patch), nasal (such as by nasal spray) and pulmonary spray.
39. The method, the combination for use, or the use of paragraph 38, wherein the GlP agonist is administered by subcutaneous injection.
40. The method, the combination for use, or the use of any one of paragraphs 1-39, wherein the GlP agonist is administered to the subject at a dose of up to about 15 mg, up to about 12.5 mg, up to about 10 mg, up to about 7.5 mg, up to about 5 mg, up to about 2.5 mg, up to about 2 mg, up to about 1 mg, up to about 0.5 mg or up to about 0.1 mg.
41. The method, the combination for use, or the use of any one of paragraphs 1-40, wherein the GlP agonist is administered to the subject at a dose of from about 10 to about 500 nmol/kg body weight, such as from about 50 to about 500 nmol/kg body weight, from about 100 to about 500 nmol/kg body weigh, or from about 200 to about 500 nmol/kg body weight.
42. The method, the combination for use, or the use of any one of paragraphs 1-41, wherein the GlP agonist is administered to the subject at a dose of about 15 mg, about 12.5 mg, about 10 mg, about 7.5 mg, about 5 mg, about 2.5 mg, or about 1 mg.
43. The method, the combination for use, or the use of any one of paragraphs 1-42, wherein the GlP agonist is administered to the subject at least once, twice, or three times daily, or once every 2 days, every 3 days, every 4 days, every 5 days, every 6 days, every 7 days, every 8 days, every 9 days, every 10 days, every 11 days, every 12 days, every 13 days, every 14 days, every 28 days or every month.
44. The method, the combination for use, or the use of paragraph 43, wherein the GlP agonist is administered to the subject once daily.
45. The method, the combination for use, or the use of paragraph 44, wherein the GlP agonist is administered to the subject once weekly.

### ADDITIONAL NUMBERED PARAGRAPHS

Other aspects of the present invention are now described by way of additional numbered paragraphs.
1. A method of increasing weight loss in a subject in need thereof, the method comprising administering to the subject a GLP-1 agonist and a GlP agonist, each in an amount that together is sufficient to increase weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.
2. A method of maintaining weight loss in a subject in need thereof, the method comprising administering to the subject a GLP-1 agonist and a GlP agonist, each in an amount that together is sufficient to maintain the weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.
3. A method of increasing weight loss in a subject in need thereof, the method comprising administering to the subject a GLP-1 agonist and a GlP agonist, each in an amount that together is sufficient to achieve additional weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.
4. A method of achieving additional weight loss in a subject in need thereof, the method comprising administering to the subject a GLP-1 agonist and a GlP agonist, each in an amount that together is sufficient to achieve the additional weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.
5. A combination of a GLP-1 agonist and a GlP agonist for use in achieving weight loss in a subject, wherein weight loss in the subject is achieved by use of the GLP-1 agonist and the GlP agonist in a method, wherein the method is as defined in any one of additional paragraphs 1-4, and wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.
6. Use of a combination of a GLP-1 agonist and a GlP agonist for manufacture of a medicament for use in a method of achieving weight loss in a subject, wherein the method is as defined in any one of additional paragraphs 1-4, and wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.
7. The method, the combination for use, or the use of any one of additional paragraphs 1-6, wherein the subject has a BMI of >30 kg/m², such as an initial BMI of >30 kg/m2 before the method is used on the subject.
8. The method, the combination for use, or the use of any one of additional paragraphs 1-7, wherein the GLP-1 agonist is selected from exenatide, liraglutide, semaglutide, dulaglutide, lixisenatide, albiglutide, and taspoglutide, preferably wherein the GLP-1 agonist is semaglutide.
9. The method, the combination for use, or the use of any one of additional paragraphs 1-8, wherein the GLP-1 agonist and/or the GlP agonist is administered by one or more of the following routes of administration: injection (including subcutaneous injection, intramuscular injection, intravenous injection, epidural injection and intraperitoneal injection), infusion pump (such as subcutaneous infusion pump), oral, sublingual, rectal, vaginal, parenteral, topical, dermal (such as by transdermal patch), nasal (such as by nasal spray) and pulmonary spray, preferably by subcutaneous injection.
10. The method, the combination for use, or the use of any one of additional paragraphs 1-9, wherein the GLP-1 agonist is administered at a dose of up to about 50 mg, such as up to about 40 mg, up to about 30 mg, up to about 20 mg or up to about 10 mg.
11. The method, the combination for use, or the use of any one of additional paragraphs 1-10, wherein the GLP-1 agonist is administered to the subject at least once, twice, or three times daily, or once every 2 days, every 3 days, every 4 days, every 5 days, every 6 days, every 7 days, every 8 days, every 9 days, every 10 days, every 11 days, every 12 days, every 13 days, every 14 days, every 28 days or every month, preferably wherein the GLP-1 agonist is administered to the subject once weekly.
12. The method, the combination for use, or the use of any one of additional paragraphs 1-11, wherein the GlP agonist is administered to the subject at a dose of up to about 15 mg, up to about 12.5 mg, up to about 10 mg, up to about 7.5 mg, up to about 5 mg, up to about 2.5 mg, up to about 2 mg, up to about 1 mg, up to about 0.5 mg or up to about 0.1 mg.
13. The method, the combination for use, or the use of any one of additional paragraphs 1-12, wherein the GlP agonist is administered to the subject at least once, twice, or three times daily, or once every 2 days, every 3 days, every 4 days, every 5 days, every 6 days, every 7 days, every 8 days, every 9 days, every 10 days, every 11 days, every 12 days, every 13 days, every 14 days, every 28 days or every month, preferably wherein the GlP agonist is administered to the subject once daily or once weekly.

### EXAMPLES

The examples described herein serve to illustrate the present invention, and the invention is not limited to the examples given.

### Example 1. Anti-Obesity Effects of GlP Analogue Compound 41 in Combination with Semaglutide in DIO Mice

Current therapies for weight management are often associated with inadequate efficacy or complications and side effects. Therefore, combination therapy with complementary modes of action is warranted to maximize the effect on weight management while maintaining safety and tolerability. Compound 41 is a long-acting gastric inhibitory peptide (GlP) analogue with a predicted half-life supporting once-weekly dosing in humans. Here we investigated the combined anti-obesity effects of compound 41 and semaglutide, once-weekly glucagon-like peptide 1 (GLP-1) analogue in diet induced obese (DIO) mice. Compound 41 and semaglutide were administered by subcutaneous injection.

### Materials

Compound 41 is a GlP analogue described above.

Semaglutide is a GLP-1 receptor agonist.

A DIO (diet induced obese) mouse is a C₅₇BL/6-“Black 6”-mouse which has been made obese through conditioning with a special diet.

### Methods

DIO mice were treated for 2 weeks by sc injection either with vehicle (qd), compound 41 (300 nmol/kg, ev. 2nd day), semaglutide (Sema, 10 nmol/kg, qd) or combination of sema+compound 41 (10 nmol/kg, qd and 300 nmol/kg, ev. 2nd, respectively). From day 14 to day 34, mice dosed with vehicle either continued on vehicle or switched to compound 41, mice dosed with compound 41 continued on compound 41 therapy, mice dosed with sema continued on sema or switched to the combination therapy of sema+compound 41. Mice dosed with combination therapy of sema+compound 41 continued on the combination therapy.

### Results

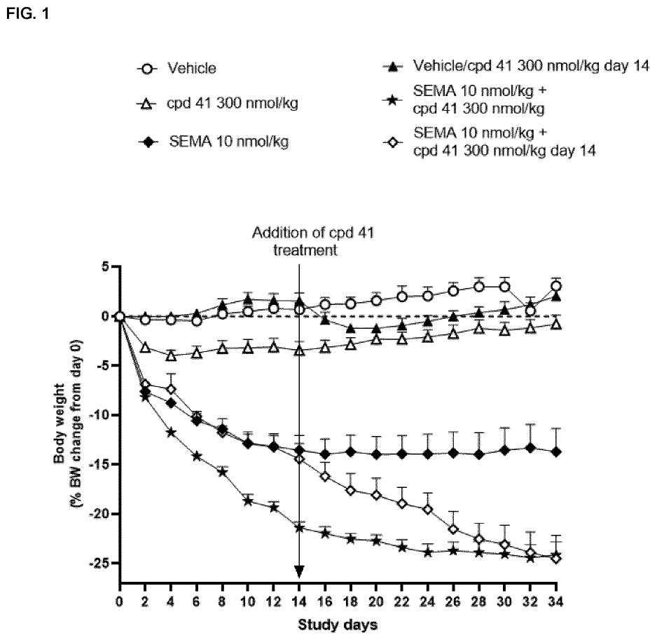

Combination therapy of sema+compound 41, achieved a significant, sustained and greater body weight reduction in DIO mice compared to vehicle- and each monotherapy group (−24.5%±2.4 combination therapy from day 14; −24.1%±1.3 combination therapy from day 0; −0.8%±0.9 compound 41 from day 0;
2.0%±0.8 compound 41 from day 14; −13.7%±2.3 sema; 3.1%±0.8, vehicle; relative to initial body weights). The effects on body weight loss in combination groups of sema+compound 41 were similar at the end of the study irrespective of the pretreatment with sema. The results are depicted in FIG. 1.

### Conclusions

In DIO mice compound 41 potentiated the benefits of GLP-1 monotherapy on body weight loss despite compound 41 alone having negligible effect at the doses tested, suggesting the greater potential of this combination therapy in treating obesity and obesity-related comorbidities, especially in subjects where the weight loss plateaus, which may be due to for example the subjects becoming GLP-1 treatment resistant.

### OTHER EMBODIMENTS

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

Other embodiments are in the claims.

What is claimed is:

1. A method of increasing weight loss in a subject in need thereof, the method comprising:
(i) providing a subject undergoing treatment with a GLP-1 agonist for a period of time during which weight loss in the subject plateaus and/or the subject is or becomes GLP-1 treatment resistant, and
(ii) following step (i), administering to the subject (a) a GLP-1 agonist and (b) a GlP agonist, each in an amount that together is sufficient to increase weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

2. A method of increasing weight loss in a subject in need thereof, the method comprising:
(i) providing a subject who fails to respond to treatment with a GLP-1 agonist, and
(ii) administering to the subject (a) a GLP-1 agonist and (b) a GlP agonist, each in an amount that together is sufficient to increase weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

3. A method of increasing weight loss in a subject in need thereof, the method comprising:
(i) providing a subject undergoing treatment with a GLP-1 agonist for a period of at least 1 month, and
(ii) following step (i), administering to the subject (a) a GLP-1 agonist and (b) a GlP agonist, each in an amount that together is sufficient to increase weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

4. A method of maintaining weight loss in a subject in need thereof, the method comprising:
(i) providing a subject undergoing treatment with a GLP-1 agonist, wherein the subject has experienced weight loss, and
(ii) following step (i), administering to the subject (a) a GLP-1 agonist and (b) a GlP agonist, each in an amount that together is sufficient to maintain the weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

5. A method of increasing weight loss in a subject in need thereof, the method comprising:
(i) providing a subject undergoing treatment with a GLP-1 agonist for a period of time during which weight loss in the subject plateaus and/or the subject is or becomes GLP-1 treatment resistant and there is a need to achieve additional weight loss in the subject, and
(ii) following step (i), administering to the subject (a) a GLP-1 agonist and (b) a GlP agonist, each in an amount that together is sufficient to achieve the additional weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

6. A method of achieving additional weight loss in a subject in need thereof, the method comprising:
(i) providing a subject undergoing treatment with a GLP-1 agonist for a period of time during which weight loss is achieved and there is a need to achieve additional weight loss in the subject, and
(ii) following step (i), administering to the subject (a) a GLP-1 agonist and (b) a GlP agonist, each in an amount that together is sufficient to achieve the additional weight loss in the subject, wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

7. A combination of a GLP-1 agonist and a GlP agonist for use in achieving weight loss in a subject, wherein weight loss in the subject is achieved by use of the GLP-1 agonist and the GlP agonist in a method, wherein the method as defined in any one of claims 1-6, and wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

8. Use of a combination of a GLP-1 agonist and a GlP agonist for manufacture of a medicament for use in a method of achieving weight loss in a subject, wherein the method is as defined in any one of claims 1-6, and wherein the GlP agonist is H—Y-Aib-EGTFISDYSIELDK-K((19-Carboxy-nonadecanoyl)-[(Piperazine-1-yl)-acetyl]-Peg3-Peg3)-AAQAFIEWLLAQGPSSGAPPPS-NH₂ (compound 41) (SEQ ID NO: 41), or a pharmaceutically acceptable salt thereof.

9. The method, the combination for use, or the use of any one of claims 1-8, wherein the subject has a BMI of >30 kg/m2, such as an initial BMI of >30 kg/m2 before the method is used on the subject.

10. The method, the combination for use, or the use of any one of claims 1-9, wherein the GLP-1 agonist in step (ii) is the same as the GLP-1 agonist in step (i).

11. The method, the combination for use, or the use of any one of claims 1-10, wherein the GLP-1 agonist is selected from exenatide, liraglutide, semaglutide, dulaglutide, lixisenatide, albiglutide, and taspoglutide, preferably wherein the GLP-1 agonist is semaglutide.

12. The method, the combination for use, or the use of any one of claims 1-11, wherein the GLP-1 agonist and/or the GlP agonist is administered by one or more of the following routes of administration: injection (including subcutaneous injection, intramuscular injection, intravenous injection, epidural injection and intraperitoneal injection), infusion pump (such as subcutaneous infusion pump), oral, sublingual, rectal, vaginal, parenteral, topical, dermal (such as by transdermal patch), nasal (such as by nasal spray) and pulmonary spray, preferably by subcutaneous injection.

13. The method, the combination for use, or the use of any one of claims 1-12, wherein the GLP-1 agonist is administered at a dose of up to about 50 mg, such as up to about 40 mg, up to about 30 mg, up to about 20 mg or up to about 10 mg.

14. The method, the combination for use, or the use of any one of claims 1-13, wherein the GLP-1 agonist is administered to the subject at least once, twice, or three times daily, or once every 2 days, every 3 days, every 4 days, every 5 days, every 6 days, every 7 days, every 8 days, every 9 days, every 10 days, every 11 days, every 12 days, every 13 days, every 14 days, every 28 days or every month, preferably wherein the GLP-1 agonist is administered to the subject once weekly.

15. The method, the combination for use, or the use of any one of claims 1-14, wherein
(a) in step (i) the GLP-1 agonist is administered to the subject as defined in any one of claims 12-14;
and/or
(b) in step (ii) the GLP-1 agonist is administered to the subject as defined in any one of claims 12-14.

16. The method, the combination for use, or the use of any one of claims 1-15, wherein the GlP agonist is administered to the subject at a dose of up to about 15 mg, up to about 12.5 mg, up to about 10 mg, up to about 7.5 mg, up to about 5 mg, up to about 2.5 mg, up to about 2 mg, up to about 1 mg, up to about 0.5 mg or up to about 0.1 mg.

17. The method, the combination for use, or the use of any one of claims 1-16, wherein the GlP agonist is administered to the subject at least once, twice, or three times daily, or once every 2 days, every 3 days, every 4 days, every 5 days, every 6 days, every 7 days, every 8 days, every 9 days, every 10 days, every 11 days, every 12 days, every 13 days, every 14 days, every 28 days or every month, preferably wherein the GlP agonist is administered to the subject once daily or once weekly.


SEQUENCE LISTING (118 sequences)

SEQ ID NO: 1 - protein, 39 aa, synthetic construct
  SITE 2: note = Aib
  SITE 17: note = conjugated to hexadecanoyl-isoGlu
YXEGTFISDYSIELDKKHQQDFVNWLLAQGPSSGAPPPS

SEQ ID NO: 2 - protein, 39 aa, synthetic construct
  SITE 2: note = Aib
  SITE 16: note = conjugated to Hexadecanoyl-isoGlu
YXEGTFISDYSIELDKIHQQDFVNWLLAQGPSSGAPPPS

SEQ ID NO: 3 - protein, 39 aa, synthetic construct
  SITE 2: note = Aib
  SITE 17: note = conjugated to Hexadecanoyl-isoGlu
YXEGTFISDYSIELEKKHQQDFVNWLLAQGPSSGAPPPS

SEQ ID NO: 4 - protein, 40 aa, synthetic construct
  SITE 2: note = Aib
  SITE 40: note = conjugated 19-carboxy-nonadecanoyl]-isoGlu-Peg3-Peg3
YXEGTFISDYSIELDKIHQQDFVNWLLAQGPSSGAPPPSK

SEQ ID NO: 5 - protein, 40 aa, synthetic construct; identical to SEQ ID NO: 4
  SITE 2: note = Aib
  SITE 40: note = conjugated to Hexadecanoyl-isoGlu

SEQ ID NO: 6 - protein, 30 aa, synthetic construct
  SITE 2: note = Aib
  SITE 30: note = conjugated to Hexadecanoyl-isoGlu
YXEGTFISDYSIELDKIHQQDFVNWLLAQK

SEQ ID NO: 7 - protein, 32 aa, synthetic construct
  SITE 2: note = Aib
  SITE 32: note = Hexadecanoyl-isoGlu
YXEGTFISDYSIELDKIHQQDFVNWLLAQKGK

SEQ ID NO: 8 - protein, 39 aa, synthetic construct
  SITE 2: note = Aib
  SITE 17: note = conjugated to [19-carboxy-nonadecanoyl]-isoGlu-Peg3-Peg3
YXEGTFISDYSIELDKKHQQDFVNYLLAQGPSSGAPPPS

SEQ ID NO: 9 - protein, 39 aa, synthetic construct; identical to SEQ ID NO: 1
  SITE 2: note = Aib
  SITE 17: note = conjugated to [19-carboxy-nonadecanoyl]-isoGlu-Peg3-Peg3

SEQ ID NO: 10 - protein, 39 aa, synthetic construct
  SITE 2: note = Aib
  SITE 17: note = conjugated to [19-carboxy-nonadecanoyl]-isoGlu-Peg3-Peg3
YXEGTFISDYSIELDKKAAQDFVNWLLAQGPSSGAPPPS

SEQ ID NO: 11 - protein, 39 aa, synthetic construct
  SITE 2: note = Aib
  SITE 17: note = conjugated to [19-carboxy-nonadecanoyl]-isoGlu-Peg3-Peg3
YXEGTFISDYSIELEKKAAKEFVNWLLAQGPSSGAPPPS

SEQ ID NO: 12 - protein, 39 aa, synthetic construct
  SITE 2: note = Aib
  SITE 17: note = conjugated to [19-carboxy-nonadecanoyl]-isoGlu-Peg3-Peg3
YXEGTFISDYSIELEKKAQRAFVEWLLAQGPSSGAPPPS

SEQ ID NO: 13 - protein, 40 aa, synthetic construct
  SITE 2: note = Aib
  SITE 40: note = conjugated to [19-carboxy-nonadecanoyl]-isoGlu-Peg3-Peg3
YXEGTFISDYSIELEKIAQRAFVEWLLAQGPSSGAPPPSK

SEQ ID NO: 14 - protein, 30 aa, synthetic construct
  SITE 2: note = Aib
  SITE 30: note = conjugated to [19-carboxy-nonadecanoyl]-isoGlu-Peg3-Peg3
YXEGTFISDYSIELEKIAQRAFVEWLLAQK

SEQ ID NO: 15 - protein, 38 aa, synthetic construct
  SITE 2: note = Aib
  SITE 17: note = conjugated to [19-carboxy-nonadecanoyl]-isoGlu-Peg3-Peg3
YXEGTFISDYSIELDKKAAQDFVNWLLAGPSSGAPPPS

SEQ ID NO: 16 - protein, 39 aa, synthetic construct
  SITE 2: note = Aib
  SITE 39: note = conjugated to [19-carboxy-nonadecanoyl]-isoGlu-Peg3-Peg3
YXEGTFISDYSIELDKIAAQDFVNWLLAGPSSGAPPPSK

SEQ ID NO: 17 - protein, 39 aa, synthetic construct
  SITE 2: note = Aib
  SITE 17: note = conjugated to (19-Carboxy-nonadecanoyl]-isoGlu-Peg3-Peg3
YXEGTFISDYSIELDKKAQRAFVEWLLAQGPSSGAPPPS

SEQ ID NO: 18 - protein, 39 aa, synthetic construct
  SITE 2: note = Aib
  SITE 17: note = conjugated to [19-Carboxy-nonadecanoyl]-isoGlu-Peg3-Peg3
YXEGTFISDYSIELDKKAQRAFIEWLLAQGPSSGAPPPS

SEQ ID NO: 19 - protein, 39 aa, synthetic construct; identical to SEQ ID NO: 18
  SITE 2: note = Aib
  SITE 17: note = conjugated to ((19-Carboxy-nonadecanoyl)-[(Piperazi ne-1-yl)-acetyl]-Peg3-Peg3)

SEQ ID NO: 20 - protein, 39 aa, synthetic construct; identical to SEQ ID NO: 17
  SITE 2: note = Aib
  SITE 17: note = conjugated to ((19-Carboxy-nonadecanoyl)-[(Piperazi ne-1-yl)-acetyl]-Peg3-Peg3)

SEQ ID NO: 21 - protein, 39 aa, synthetic construct
  SITE 2: note = Aib
  SITE 17: note = conjugated to ((19-Carboxy-nonadecanoyl)-[(Piperazi ne-1-yl)-acetyl]-Peg3-Peg3)
YXEGTFISDYSIELDKKAQKEFVEWLLAAGPSSGAPPPS

SEQ ID NO: 22 - protein, 39 aa, synthetic construct; identical to SEQ ID NO: 21
  SITE 2: note = Aib
  SITE 17: note = conjugated to ([19-Carboxy-nonadecanoyl]-isoGlu-Peg3-Peg3)

SEQ ID NO: 23 - protein, 39 aa, synthetic construct
  SITE 2: note = Aib
  SITE 39: note = conjugated to ([19-Carboxy-nonadecanoyl]-isoGlu-Peg3-Peg3)
YXEGTFISDYSIELDKIAQRAFIEWLLAGPSSGAPPPSK

SEQ ID NO: 24 - protein, 39 aa, synthetic construct
  SITE 2: note = Aib
  SITE 39: note = conjugated to ([19-Carboxy-nonadecanoyl]-isoGlu-Peg3-Peg3)
YXEGTFISDYSIELDKIAQKEFIEWLLAGPSSGAPPPSK

SEQ ID NO: 25 - protein, 39 aa, synthetic construct
  SITE 2: note = Aib
  SITE 39: note = conjugated to ([19-Carboxy-nonadecanoyl]-isoGlu-Peg3-Peg3)
YXEGTFISDYSIELDKIAAQDFIEWLLAGPSSGAPPPSK

SEQ ID NO: 26 - protein, 39 aa, synthetic construct; identical to SEQ ID NO: 25
  SITE 2: note = Aib
  SITE 39: note = conjugated to ((19-Carboxy-nonadecanoyl)-[(Piperazi ne-1-yl)-acetyl]-Peg3-Peg3)

SEQ ID NO: 27 - protein, 39 aa, synthetic construct
  SITE 2: note = Aib
  SITE 39: note = ([19-Carboxy-nonadecanoyl]-isoGlu-Peg3-Peg3)
YXEGTFISDYSIELDKIAAQDFVEWLLAGPSSGAPPPSK

SEQ ID NO: 28 - protein, 40 aa, synthetic construct
  SITE 2: note = Aib
  SITE 40: note = conjugated to ([19-Carboxy-nonadecanoyl]-isoGlu-Peg3-Peg3)
YXEGTFISDYSIELDKIAQRAFIEWLLAQGPSSGAPPPSK

SEQ ID NO: 29 - protein, 38 aa, synthetic construct
  SITE 2: note = Aib
  SITE 17: note = conjugated to ([19-Carboxy-nonadecanoyl]-isoGlu-Peg3-Peg3)
YXEGTFISDYSIELDKKAAQAFVNWLLAGPSSGAPPPS

SEQ ID NO: 30 - protein, 39 aa, synthetic construct
  SITE 2: note = Aib
  SITE 17: note = conjugated to ([19-Carboxy-nonadecanoyl]-isoGlu-Peg3-Peg3)
YXEGTFISDYSIELDKKAAQDFVNWLLAAGPSSGAPPPS

SEQ ID NO: 31 - protein, 38 aa, synthetic construct
  SITE 2: note = Aib
  SITE 17: note = conjugated to ([19-Carboxy-nonadecanoyl]-isoGlu-Peg3-Peg3)
YXEGTFISDYSIELDKKAAQDFINWLLAGPSSGAPPPS

SEQ ID NO: 32 - protein, 38 aa, synthetic construct
  SITE 2: note = Aib
  SITE 17: note = conjugated to ([19-Carboxy-nonadecanoyl]-isoGlu-Peg3-Peg3)
YXEGTFISDYSIELDKKAAQDFIEWLLAGPSSGAPPPS

SEQ ID NO: 33 - protein, 38 aa, synthetic construct; identical to SEQ ID NO: 32
  SITE 2: note = Aib
  SITE 17: note = conjugated to ((19-Carboxy-nonadecanoyl)-[(Piperazi ne-1-yl)-acetyl]-Peg3-Peg3)

SEQ ID NO: 34 - protein, 39 aa, synthetic construct
  SITE 2: note = Aib
  SITE 16: note = conjugated to ([19-Carboxy-nonadecanoyl]-isoGlu-Peg3-Peg3)
YXEGTFISDYSIELDKIAQRAFIEWLLAQGPSSGAPPPS

SEQ ID NO: 35 - protein, 39 aa, synthetic construct
  SITE 2: note = Aib
  SITE 12: note = conjugated to ([19-Carboxy-nonadecanoyl]-isoGlu-Peg3-Peg3)
YXEGTFISDYSKELDKIAQRAFIEWLLAQGPSSGAPPPS

SEQ ID NO: 36 - protein, 39 aa, synthetic construct
  SITE 2: note = D-Alanine
  SITE 17: note = conjugated to ([19-Carboxy-nonadecanoyl]-isoGlu-Peg3-Peg3)
YAEGTFISDYSIELDKKAQRAFIEWLLAQGPSSGAPPPS

SEQ ID NO: 37 - protein, 39 aa, synthetic construct
  SITE 2: note = D-Alanine
  SITE 39: note = conjugated to ((19-Carboxy-nonadecanoyl)-[(Piperazi ne-1-yl)-acetyl]-Peg3-Peg3)
YAEGTFISDYSIELDKIAAQDFIEWLLAGPSSGAPPPSK

SEQ ID NO: 38 - protein, 39 aa, synthetic construct
  SITE 2: note = Aib
  SITE 17: note = ([19-Carboxy-nonadecanoyl]-isoGlu-Peg3-Peg3)
YXEGTFISDYSIELDKKAAQDFIEWLLAQGPSSGAPPPS

SEQ ID NO: 39 - protein, 39 aa, synthetic construct
  SITE 2: note = Aib
  SITE 17: note = conjugated to ([19-Carboxy-nonadecanoyl]-isoGlu-Peg3-Peg3)
YXEGTFISDYSIELDKKAAQDFINWLLAQGPSSGAPPPS

SEQ ID NO: 40 - protein, 39 aa, synthetic construct
  SITE 2: note = Aib
  SITE 17: note = conjugated to ([19-Carboxy-nonadecanoyl]-isoGlu-Peg3-Peg3)
YXEGTFISDYSIELDKKAAQAFIEWLLAQGPSSGAPPPS

SEQ ID NO: 41 - protein, 39 aa, synthetic construct; identical to SEQ ID NO: 40
  SITE 2: note = Aib
  SITE 17: note = conjugated to ((19-Carboxy-nonadecanoyl)-[(Piperazi ne-1-yl)-acetyl]-Peg3-Peg3)

SEQ ID NO: 42 - protein, 32 aa, synthetic construct
  VARIANT 1: note = H-, Ac or pGlu pyroglutamic acid (pGlu; (S)-(-)-2-pyrrolidone-5-carboxylic acid), C1-4 alkyl, acetyl, formyl, benzoyl and trifluoroacetyl
  VARIANT 3: note = Aib, Ala, D-Ala, Gly, Ser, N-Me-Ser, Ac3c, Ac4c or Ac5c
  VARIANT 11: note = Tyr, Leu or Ser
  VARIANT 12: note = Ser or Leu
  VARIANT 13: note = Lys, Ile or a residue independently selected from Lys, Arg, Orn and Cys and wherein the side chain of said residue is conjugated to a lipophilic substituent
  VARIANT 16: note = Asp or Glu
  VARIANT 17: note = Ser, Glu, Lys, or a residue independently selected from Lys, Arg, Orn and Cys and wherein the side chain of said residue is conjugated to a lipophilic substituent
  VARIANT 18: note = Ile, Lys, Gln, Arg, or a residue independently selected from Lys, Arg, Orn and Cys and wherein the side chain of said residue is conjugated to a lipophilic substituent
  VARIANT 19: note = His, Arg or Ala
  VARIANT 20: note = Gln, Lys, Ala or Glu
  VARIANT 21: note = Gln, Lys, Ala, His or Arg
  VARIANT 22: note = Ala, Leu, Asp or Glu
  VARIANT 24: note = Val or Ile
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 32: note = Residue is absent or is a residue independently selected from Lys, Arg, Orn and Cys and wherein the side chain of said residue is conjugated to a lipophilic substituent
  SITE 32: note = residue further connected to NH2 or OH
  VARIANT 31: note = X is selected from the sequences Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), or Gly-Lys-Lys-Asn-Asp-Trp-Lys-His-Asn-Ile-Thr-Gln (SEQ ID NO: 47), or is absent
XYXEGTFISDXXXELXXXXXXXFXXXLXXXXX

SEQ ID NO: 43 - protein, 10 aa, synthetic construct
GPSSGAPPPS

SEQ ID NO: 44 - protein, 9 aa, synthetic construct
GPSSGAPPS

SEQ ID NO: 45 - protein, 9 aa, synthetic construct
PSSGAPPPS

SEQ ID NO: 46 - protein, 8 aa, synthetic construct
PSSGAPPS

SEQ ID NO: 47 - protein, 12 aa, synthetic construct
GKKNDWKHNITQ

SEQ ID NO: 48 - protein, 32 aa, synthetic construct
  VARIANT 1: note = H-, Ac or pGlu pyroglutamic acid
  VARIANT 3: note = Aib, Ala, D-Ala or Gly
  VARIANT 13: note = Lys, Ile, or a Lys residue wherein the side chain of said Lys residue is conjugated to a lipophilic substituent
  VARIANT 16: note = Asp or Glu
  VARIANT 17: note = Ser, Glu, Lys or a Lys residue wherein the side chain of said Lys residue is conjugated to a lipophilic substituent
  VARIANT 18: note = Ile, Lys, Gln, Arg or a Lys residue wherein the side chain of said Lys residue is conjugated to a lipophilic substituent
  VARIANT 19: note = His, Arg or Ala
  VARIANT 20: note = Gln or Ala
  VARIANT 21: note = Gln, Lys, Ala, His or Arg
  VARIANT 22: note = Ala, Asp or Glu
  VARIANT 24: note = Ile or Val
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 32: note = Residue is absent or is a Lys residue wherein the side chain of said Lys residue is conjugated to a lipophilic substituent
  SITE 32: note = residue further connected to NH2 or OH
  VARIANT 31: note = X is selected from the sequences Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), or Gly-Lys-Lys-Asn-Asp-Trp-Lys-His-Asn-Ile-Thr-Gln (SEQ ID NO: 47), or is absent
XYXEGTFISDYSXELXXXXXXXFXXXLXXXXX

SEQ ID NO: 49 - skipped

SEQ ID NO: 50 - protein, 32 aa, synthetic construct
  VARIANT 1: note = H-, Ac or pGlu pyroglutamic acid
  SITE 3: note = Aib
  VARIANT 16: note = Asp or Glu
  VARIANT 17: note = Lys or a residue independently selected from Lys, Arg, Orn and Cys and wherein the side chain of said residue is conjugated to a lipophilic substituent
  VARIANT 18: note = Ile or a residue independently selected from Lys, Arg, Orn and Cys and wherein the side chain of said residue is conjugated to a lipophilic substituent
  VARIANT 19: note = His or Ala
  VARIANT 20: note = Gln or Ala
  VARIANT 21: note = Gln, Lys or Arg
  VARIANT 22: note = Ala, Asp or Glu
  VARIANT 25: note = Asn or Glu
  VARIANT 26: note = Tyr or Trp
  VARIANT 30: note = Gln or is absent
  VARIANT 32: note = Residue is absent or is a residue independently selected from Lys, Arg, Orn and Cys and wherein the side chain of said residue is conjugated to a lipophilic substituent
  SITE 32: note = residue further connected to NH2 or OH
  VARIANT 31: note = X is selected from the sequences Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), or Gly-Lys-Lys-Asn-Asp-Trp-Lys-His-Asn-Ile-Thr-Gln (SEQ ID NO: 47), or is absent
XYXEGTFISDYSIELXXXXXXXFVXXLLAXXX

SEQ ID NO: 51 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXHQQDFVNWLLAQGPSSGAPPPS

SEQ ID NO: 52 - protein, 39 aa, synthetic construct
  SITE 2: note = Aib
  VARIANT 16: note = residue independently selected from Lys, Arg, Orn and Cys and wherein the side chain of said residue is conjugated to a lipophilic substituent
YXEGTFISDYSIELDXIHQQDFVNWLLAQGPSSGAPPPS

SEQ ID NO: 53 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELEKXHQQDFVNWLLAQGPSSGAPPPS

SEQ ID NO: 54 - protein, 40 aa, synthetic construct
  SITE 2: note = Aib
  VARIANT 40: note = residue independently selected from Lys, Arg, Orn and Cys and wherein the side chain of said residue is conjugated to a lipophilic substituent
YXEGTFISDYSIELDKIHQQDFVNWLLAQGPSSGAPPPSX

SEQ ID NO: 55 - protein, 30 aa, synthetic construct
  SITE 2: note = Aib
  VARIANT 30: note = residue independently selected from Lys, Arg, Orn and Cys and wherein the side chain of said residue is conjugated to a lipophilic substituent
YXEGTFISDYSIELDKIHQQDFVNWLLAQX

SEQ ID NO: 56 - protein, 32 aa, synthetic construct
  SITE 2: note = Aib
  VARIANT 32: note = residue independently selected from Lys, Arg, Orn and Cys and wherein the side chain of said residue is conjugated to a lipophilic substituent
YXEGTFISDYSIELDKIHQQDFVNWLLAQKGX

SEQ ID NO: 57 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXHQQDFVNYLLAQGPSSGAPPPS

SEQ ID NO: 58 - protein, 39 aa, synthetic construct; identical to SEQ ID NO: 51
  SITE 2: note = Aib
  VARIANT 17: note = residue independently selected from Lys, Arg, Orn and Cys and wherein the side chain of said residue is conjugated to a lipophilic substituent

SEQ ID NO: 59 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQDFVNWLLAQGPSSGAPPPS

SEQ ID NO: 60 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELEKXAAKEFVNWLLAQGPSSGAPPPS

SEQ ID NO: 61 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELEKXAQRAFVEWLLAQGPSSGAPPPS

SEQ ID NO: 62 - protein, 40 aa, synthetic construct
  SITE 2: note = Aib
  VARIANT 40: note = residue independently selected from Lys, Arg, Orn and Cys and wherein the side chain of said residue is conjugated to a lipophilic substituent
YXEGTFISDYSIELEKIAQRAFVEWLLAQGPSSGAPPPSX

SEQ ID NO: 63 - protein, 30 aa, synthetic construct
  SITE 2: note = Aib
  VARIANT 30: note = residue independently selected from Lys, Arg, Orn and Cys and wherein the side chain of said residue is conjugated to a lipophilic substituent
YXEGTFISDYSIELEKIAQRAFVEWLLAQX

SEQ ID NO: 64 - protein, 38 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQDFVNWLLAGPSSGAPPPS

SEQ ID NO: 65 - protein, 39 aa, synthetic construct
  SITE 2: note = Aib
  VARIANT 39: note = residue independently selected from Lys, Arg, Orn and Cys and wherein the side chain of said residue is conjugated to a lipophilic substituent
YXEGTFISDYSIELDKIAAQDFVNWLLAGPSSGAPPPSX

SEQ ID NO: 66 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAQRAFVEWLLAQGPSSGAPPPS

SEQ ID NO: 67 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAQRAFIEWLLAQGPSSGAPPPS

SEQ ID NO: 68 - protein, 40 aa, synthetic construct
  SITE 2: note = Aib
  VARIANT 40: note = residue independently selected from Lys, Arg, Orn and Cys and wherein the side chain of said residue is conjugated to a lipophilic substituent
YXEGTFISDYSIELDKIAQRAFIEWLLAGPSSGAPPPSKX

SEQ ID NO: 69 - protein, 40 aa, synthetic construct
  SITE 2: note = Aib
  VARIANT 40: note = residue independently selected from Lys, Arg, Orn and Cys and wherein the side chain of said residue is conjugated to a lipophilic substituent
YXEGTFISDYSIELDKIAQKEFIEWLLAGPSSGAPPPSKX

SEQ ID NO: 70 - protein, 40 aa, synthetic construct
  SITE 2: note = Aib
  VARIANT 40: note = residue independently selected from Lys, Arg, Orn and Cys and wherein the side chain of said residue is conjugated to a lipophilic substituent
YXEGTFISDYSIELDKIAAQDFIEWLLAGPSSGAPPPSKX

SEQ ID NO: 71 - protein, 40 aa, synthetic construct
  SITE 2: note = Aib
  VARIANT 40: note = residue independently selected from Lys, Arg, Orn and Cys and wherein the side chain of said residue is conjugated to a lipophilic substituent
YXEGTFISDYSIELDKIAAQDFVEWLLAGPSSGAPPPSKX

SEQ ID NO: 72 - protein, 41 aa, synthetic construct
  SITE 2: note = Aib
  VARIANT 41: note = residue independently selected from Lys, Arg, Orn and Cys and wherein the side chain of said residue is conjugated to a lipophilic substituent
YXEGTFISDYSIELDKIAQRAFIEWLLAQGPSSGAPPPSKX

SEQ ID NO: 73 - protein, 38 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQAFVNWLLAGPSSGAPPPS

SEQ ID NO: 74 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQDFVNWLLAAGPSSGAPPPS

SEQ ID NO: 75 - protein, 38 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQDFINWLLAGPSSGAPPPS

SEQ ID NO: 76 - protein, 38 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQDFIEWLLAGPSSGAPPPS

SEQ ID NO: 77 - protein, 38 aa, synthetic construct; identical to SEQ ID NO: 76
  SITE 2: note = Aib
  VARIANT 17: note = residue independently selected from Lys, Arg, Orn and Cys and wherein the side chain of said residue is conjugated to a lipophilic substituent

SEQ ID NO: 78 - protein, 40 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXIAQRAFIEWLLAQGPSSGAPPPS

SEQ ID NO: 79 - protein, 39 aa, synthetic construct
  SITE 2: note = Aib
  VARIANT 12: note = residue independently selected from Lys, Arg, Orn and Cys and wherein the side chain of said residue is conjugated to a lipophilic substituent
YXEGTFISDYSXELDKIAQRAFIEWLLAQGPSSGAPPPS

SEQ ID NO: 80 - protein, 39 aa, synthetic construct
  SITE 2: note = D-Alanine
  VARIANT 17: note = residue independently selected from Lys, Arg, Orn and Cys and wherein the side chain of said residue is conjugated to a lipophilic substituent
YAEGTFISDYSIELDKXAQRAFIEWLLAQGPSSGAPPPS

SEQ ID NO: 81 - protein, 40 aa, synthetic construct
  SITE 2: note = D-Alanine
  VARIANT 40: note = residue independently selected from Lys, Arg, Orn and Cys and wherein the side chain of said residue is conjugated to a lipophilic substituent
YAEGTFISDYSIELDKIAAQDFIEWLLAGPSSGAPPPSKX

SEQ ID NO: 82 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQDFIEWLLAQGPSSGAPPPS

SEQ ID NO: 83 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQDFINWLLAQGPSSGAPPPS

SEQ ID NO: 84 - protein, 39 aa, synthetic construct
  SITE 2: note = Aib
  VARIANT 17: note = residue independently selected from Lys, Arg, Orn and Cys and wherein the side chain of said residue is conjugated to a lipophilic substituent
YXEGTFISDYSIELDKXAAQAFIEWLLAQGPSSGAPPPS

SEQ ID NO: 85 - protein, 6 aa, synthetic construct
GSGSGG

SEQ ID NO: 86-88 - skipped (3 entries)

SEQ ID NO: 89 - protein, 38 aa, synthetic construct
HGEGTFTSDLSKQMEEEAVRLFIEWLKNGGPSSGAPPP

SEQ ID NO: 90 - protein, 31 aa, synthetic construct
HAEGTFTSDVSSYLEGQAAKEFIAWLVRGRG

SEQ ID NO: 91 - protein, 31 aa, synthetic construct
  SITE 2: note = Aib
  SITE 20: note = side-chain amino group of the lysine at position 20 is linked to the lipohilic moiety [2-(2-[2-(2-[2-(2-[4-(17 -Carboxyheptadecanoylamino)-4(S)-carboxybutyrylamino]ethox y)ethoxy]acetylamino)ethoxy]ethoxy)acetyl]
HXEGTFTSDVSSYLEGQAAKEFIAWLVRGRG

SEQ ID NO: 92 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 93 - protein, 44 aa, synthetic construct
HGEGTFTSDLSKQMEEEAVRLFIEWLKNGGPSSGAPPSKKKKKK

SEQ ID NO: 94 - protein, 645 aa, synthetic construct
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 95 - protein, 30 aa, synthetic construct
  SITE 2: note = Aib
  SITE 29: note = Aib
HXEGTFTSDVSSYLEGQAAKEFIAWLVKXR

SEQ ID NO: 96 - protein, 40 aa, synthetic construct; identical to SEQ ID NO: 54
  SITE 2: note = Aib
  VARIANT 40: note = residue independently selected from Lys, Arg, Orn and Cys and wherein the side chain of said residue is conjugated to a lipophilic substituent

SEQ ID NO: 97 - protein, 41 aa, synthetic construct
  VARIANT 1: note = H-, Ac or pGlu pyroglutamic acid (pGlu; (S)-(-)-2-pyrrolidone-5-carboxylic acid), C1-4 alkyl, acetyl, formyl, benzoyl and trifluoroacetyl
  VARIANT 3: note = Aib, Ala, D-Ala, Gly, Ser, N-Me-Ser, Ac3c, Ac4c or Ac5c
  VARIANT 11: note = Tyr, Leu or Ser
  VARIANT 12: note = Ser or Leu
  VARIANT 13: note = Lys, Ile or a residue independently selected from Lys, Arg, Orn and Cys and wherein the side chain of said residue is conjugated to a lipophilic substituent
  VARIANT 16: note = Asp or Glu
  VARIANT 17: note = Ser, Glu, Lys, or a residue independently selected from Lys, Arg, Orn and Cys and wherein the side chain of said residue is conjugated to a lipophilic substituent
  VARIANT 18: note = Ile, Lys, Gln, Arg, or a residue independently selected from Lys, Arg, Orn and Cys and wherein the side chain of said residue is conjugated to a lipophilic substituent
  VARIANT 19: note = His, Arg or Ala
  VARIANT 20: note = Gln, Lys, Ala or Glu
  VARIANT 21: note = Gln, Lys, Ala, His or Arg
  VARIANT 22: note = Ala, Leu, Asp or Glu
  VARIANT 24: note = Val or Ile
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 41: note = Residue is absent or is a residue independently selected from Lys, Arg, Orn and Cys and wherein the side chain of said residue is conjugated to a lipophilic substituent
  SITE 41: note = residue further connected to NH2 or OH
XYXEGTFISDXXXELXXXXXXXFXXXLXXXGPSSGAPPPSX

SEQ ID NO: 98 - protein, 40 aa, synthetic construct
  VARIANT 1: note = H-, Ac or pGlu pyroglutamic acid (pGlu; (S)-(-)-2-pyrrolidone-5-carboxylic acid), C1-4 alkyl, acetyl, formyl, benzoyl and trifluoroacetyl
  VARIANT 3: note = Aib, Ala, D-Ala, Gly, Ser, N-Me-Ser, Ac3c, Ac4c or Ac5c
  VARIANT 11: note = Tyr, Leu or Ser
  VARIANT 12: note = Ser or Leu
  VARIANT 13: note = Lys, Ile or a residue independently selected from Lys, Arg, Orn and Cys and wherein the side chain of said residue is conjugated to a lipophilic substituent
  VARIANT 16: note = Asp or Glu
  VARIANT 17: note = Ser, Glu, Lys, or a residue independently selected from Lys, Arg, Orn and Cys and wherein the side chain of said residue is conjugated to a lipophilic substituent
  VARIANT 18: note = Ile, Lys, Gln, Arg, or a residue independently selected from Lys, Arg, Orn and Cys and wherein the side chain of said residue is conjugated to a lipophilic substituent
  VARIANT 19: note = His, Arg or Ala
  VARIANT 20: note = Gln, Lys, Ala or Glu
  VARIANT 21: note = Gln, Lys, Ala, His or Arg
  VARIANT 22: note = Ala, Leu, Asp or Glu
  VARIANT 24: note = Val or Ile
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 40: note = Residue is absent or is a residue independently selected from Lys, Arg, Orn and Cys and wherein the side chain of said residue is conjugated to a lipophilic substituent
  SITE 40: note = residue further connected to NH2 or OH
XYXEGTFISDXXXELXXXXXXXFXXXLXXXGPSSGAPPSX

SEQ ID NO: 99 - protein, 40 aa, synthetic construct
  VARIANT 1: note = H-, Ac or pGlu pyroglutamic acid (pGlu; (S)-(-)-2-pyrrolidone-5-carboxylic acid), C1-4 alkyl, acetyl, formyl, benzoyl and trifluoroacetyl
  VARIANT 3: note = Aib, Ala, D-Ala, Gly, Ser, N-Me-Ser, Ac3c, Ac4c or Ac5c
  VARIANT 11: note = Tyr, Leu or Ser
  VARIANT 12: note = Ser or Leu
  VARIANT 13: note = Lys, Ile or a residue independently selected from Lys, Arg, Orn and Cys and wherein the side chain of said residue is conjugated to a lipophilic substituent
  VARIANT 16: note = Asp or Glu
  VARIANT 17: note = Ser, Glu, Lys, or a residue independently selected from Lys, Arg, Orn and Cys and wherein the side chain of said residue is conjugated to a lipophilic substituent
  VARIANT 18: note = Ile, Lys, Gln, Arg, or a residue independently selected from Lys, Arg, Orn and Cys and wherein the side chain of said residue is conjugated to a lipophilic substituent
  VARIANT 19: note = His, Arg or Ala
  VARIANT 20: note = Gln, Lys, Ala or Glu
  VARIANT 21: note = Gln, Lys, Ala, His or Arg
  VARIANT 22: note = Ala, Leu, Asp or Glu
  VARIANT 24: note = Val or Ile
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 40: note = Residue is absent or is a residue independently selected from Lys, Arg, Orn and Cys and wherein the side chain of said residue is conjugated to a lipophilic substituent
  SITE 40: note = residue further connected to NH2 or OH
XYXEGTFISDXXXELXXXXXXXFXXXLXXXPSSGAPPPSX

SEQ ID NO: 100 - protein, 39 aa, synthetic construct
  VARIANT 1: note = H-, Ac or pGlu pyroglutamic acid (pGlu; (S)-(-)-2-pyrrolidone-5-carboxylic acid), C1-4 alkyl, acetyl, formyl, benzoyl and trifluoroacetyl
  VARIANT 3: note = Aib, Ala, D-Ala, Gly, Ser, N-Me-Ser, Ac3c, Ac4c or Ac5c
  VARIANT 11: note = Tyr, Leu or Ser
  VARIANT 12: note = Ser or Leu
  VARIANT 13: note = Lys, Ile or a residue independently selected from Lys, Arg, Orn and Cys and wherein the side chain of said residue is conjugated to a lipophilic substituent
  VARIANT 16: note = Asp or Glu
  VARIANT 17: note = Ser, Glu, Lys, or a residue independently selected from Lys, Arg, Orn and Cys and wherein the side chain of said residue is conjugated to a lipophilic substituent
  VARIANT 18: note = Ile, Lys, Gln, Arg, or a residue independently selected from Lys, Arg, Orn and Cys and wherein the side chain of said residue is conjugated to a lipophilic substituent
  VARIANT 19: note = His, Arg or Ala
  VARIANT 20: note = Gln, Lys, Ala or Glu
  VARIANT 21: note = Gln, Lys, Ala, His or Arg
  VARIANT 22: note = Ala, Leu, Asp or Glu
  VARIANT 24: note = Val or Ile
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 39: note = Residue is absent or is a residue independently selected from Lys, Arg, Orn and Cys and wherein the side chain of said residue is conjugated to a lipophilic substituent
  SITE 39: note = residue further connected to NH2 or OH
XYXEGTFISDXXXELXXXXXXXFXXXLXXXPSSGAPPSX

SEQ ID NO: 101 - protein, 43 aa, synthetic construct
  VARIANT 1: note = H-, Ac or pGlu pyroglutamic acid (pGlu; (S)-(-)-2-pyrrolidone-5-carboxylic acid), C1-4 alkyl, acetyl, formyl, benzoyl and trifluoroacetyl
  VARIANT 3: note = Aib, Ala, D-Ala, Gly, Ser, N-Me-Ser, Ac3c, Ac4c or Ac5c
  VARIANT 11: note = Tyr, Leu or Ser
  VARIANT 12: note = Ser or Leu
  VARIANT 13: note = Lys, Ile or a residue independently selected from Lys, Arg, Orn and Cys and wherein the side chain of said residue is conjugated to a lipophilic substituent
  VARIANT 16: note = Asp or Glu
  VARIANT 17: note = Ser, Glu, Lys, or a residue independently selected from Lys, Arg, Orn and Cys and wherein the side chain of said residue is conjugated to a lipophilic substituent
  VARIANT 18: note = Ile, Lys, Gln, Arg, or a residue independently selected from Lys, Arg, Orn and Cys and wherein the side chain of said residue is conjugated to a lipophilic substituent
  VARIANT 19: note = His, Arg or Ala
  VARIANT 20: note = Gln, Lys, Ala or Glu
  VARIANT 21: note = Gln, Lys, Ala, His or Arg
  VARIANT 22: note = Ala, Leu, Asp or Glu
  VARIANT 24: note = Val or Ile
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 43: note = Residue is absent or is a residue independently selected from Lys, Arg, Orn and Cys and wherein the side chain of said residue is conjugated to a lipophilic substituent
  SITE 43: note = residue further connected to NH2 or OH
XYXEGTFISDXXXELXXXXXXXFXXXLXXXGKKNDWKHNITQX

SEQ ID NO: 102 - protein, 31 aa, synthetic construct
  VARIANT 1: note = H-, Ac or pGlu pyroglutamic acid (pGlu; (S)-(-)-2-pyrrolidone-5-carboxylic acid), C1-4 alkyl, acetyl, formyl, benzoyl and trifluoroacetyl
  VARIANT 3: note = Aib, Ala, D-Ala, Gly, Ser, N-Me-Ser, Ac3c, Ac4c or Ac5c
  VARIANT 11: note = Tyr, Leu or Ser
  VARIANT 12: note = Ser or Leu
  VARIANT 13: note = Lys, Ile or a residue independently selected from Lys, Arg, Orn and Cys and wherein the side chain of said residue is conjugated to a lipophilic substituent
  VARIANT 16: note = Asp or Glu
  VARIANT 17: note = Ser, Glu, Lys, or a residue independently selected from Lys, Arg, Orn and Cys and wherein the side chain of said residue is conjugated to a lipophilic substituent
  VARIANT 18: note = Ile, Lys, Gln, Arg, or a residue independently selected from Lys, Arg, Orn and Cys and wherein the side chain of said residue is conjugated to a lipophilic substituent
  VARIANT 19: note = His, Arg or Ala
  VARIANT 20: note = Gln, Lys, Ala or Glu
  VARIANT 21: note = Gln, Lys, Ala, His or Arg
  VARIANT 22: note = Ala, Leu, Asp or Glu
  VARIANT 24: note = Val or Ile
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 31: note = Residue is absent or is a residue independently selected from Lys, Arg, Orn and Cys and wherein the side chain of said residue is conjugated to a lipophilic substituent
  SITE 31: note = residue further connected to NH2 or OH
XYXEGTFISDXXXELXXXXXXXFXXXLXXXX

SEQ ID NO: 103 - protein, 41 aa, synthetic construct
  VARIANT 1: note = H-, Ac or pGlu pyroglutamic acid
  VARIANT 3: note = Aib, Ala, D-Ala or Gly
  VARIANT 13: note = Lys, Ile, or a Lys residue wherein the side chain of said Lys residue is conjugated to a lipophilic substituent
  VARIANT 16: note = Asp or Glu
  VARIANT 17: note = Ser, Glu, Lys or a Lys residue wherein the side chain of said Lys residue is conjugated to a lipophilic substituent
  VARIANT 18: note = Ile, Lys, Gln, Arg or a Lys residue wherein the side chain of said Lys residue is conjugated to a lipophilic substituent
  VARIANT 19: note = His, Arg or Ala
  VARIANT 20: note = Gln or Ala
  VARIANT 21: note = Gln, Lys, Ala, His or Arg
  VARIANT 22: note = Ala, Asp or Glu
  VARIANT 24: note = Ile or Val
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 41: note = Residue is absent or is a Lys residue wherein the side chain of said Lys residue is conjugated to a lipophilic substituent
  SITE 41: note = residue further connected to NH2 or OH
XYXEGTFISDYSXELXXXXXXXFXXXLXXXGPSSGAPPPSX

SEQ ID NO: 104 - protein, 40 aa, synthetic construct
  VARIANT 1: note = H-, Ac or pGlu pyroglutamic acid
  VARIANT 3: note = Aib, Ala, D-Ala or Gly
  VARIANT 13: note = Lys, Ile, or a Lys residue wherein the side chain of said Lys residue is conjugated to a lipophilic substituent
  VARIANT 16: note = Asp or Glu
  VARIANT 17: note = Ser, Glu, Lys or a Lys residue wherein the side chain of said Lys residue is conjugated to a lipophilic substituent
  VARIANT 18: note = Ile, Lys, Gln, Arg or a Lys residue wherein the side chain of said Lys residue is conjugated to a lipophilic substituent
  VARIANT 19: note = His, Arg or Ala
  VARIANT 20: note = Gln or Ala
  VARIANT 21: note = Gln, Lys, Ala, His or Arg
  VARIANT 22: note = Ala, Asp or Glu
  VARIANT 24: note = Ile or Val
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 40: note = Residue is absent or is a Lys residue wherein the side chain of said Lys residue is conjugated to a lipophilic substituent
  SITE 40: note = residue further connected to NH2 or OH
XYXEGTFISDYSXELXXXXXXXFXXXLXXXGPSSGAPPSX

SEQ ID NO: 105 - protein, 40 aa, synthetic construct
  VARIANT 1: note = H-, Ac or pGlu pyroglutamic acid
  VARIANT 3: note = Aib, Ala, D-Ala or Gly
  VARIANT 13: note = Lys, Ile, or a Lys residue wherein the side chain of said Lys residue is conjugated to a lipophilic substituent
  VARIANT 16: note = Asp or Glu
  VARIANT 17: note = Ser, Glu, Lys or a Lys residue wherein the side chain of said Lys residue is conjugated to a lipophilic substituent
  VARIANT 18: note = Ile, Lys, Gln, Arg or a Lys residue wherein the side chain of said Lys residue is conjugated to a lipophilic substituent
  VARIANT 19: note = His, Arg or Ala
  VARIANT 20: note = Gln or Ala
  VARIANT 21: note = Gln, Lys, Ala, His or Arg
  VARIANT 22: note = Ala, Asp or Glu
  VARIANT 24: note = Ile or Val
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 40: note = Residue is absent or is a Lys residue wherein the side chain of said Lys residue is conjugated to a lipophilic substituent
  SITE 40: note = residue further connected to NH2 or OH
XYXEGTFISDYSXELXXXXXXXFXXXLXXXPSSGAPPPSX

SEQ ID NO: 106 - protein, 39 aa, synthetic construct
  VARIANT 1: note = H-, Ac or pGlu pyroglutamic acid
  VARIANT 3: note = Aib, Ala, D-Ala or Gly
  VARIANT 13: note = Lys, Ile, or a Lys residue wherein the side chain of said Lys residue is conjugated to a lipophilic substituent
  VARIANT 16: note = Asp or Glu
  VARIANT 17: note = Ser, Glu, Lys or a Lys residue wherein the side chain of said Lys residue is conjugated to a lipophilic substituent
  VARIANT 18: note = Ile, Lys, Gln, Arg or a Lys residue wherein the side chain of said Lys residue is conjugated to a lipophilic substituent
  VARIANT 19: note = His, Arg or Ala
  VARIANT 20: note = Gln or Ala
  VARIANT 21: note = Gln, Lys, Ala, His or Arg
  VARIANT 22: note = Ala, Asp or Glu
  VARIANT 24: note = Ile or Val
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 39: note = Residue is absent or is a Lys residue wherein the side chain of said Lys residue is conjugated to a lipophilic substituent
  SITE 39: note = residue further connected to NH2 or OH
XYXEGTFISDYSXELXXXXXXXFXXXLXXXPSSGAPPSX

SEQ ID NO: 107 - protein, 43 aa, synthetic construct
  VARIANT 1: note = H-, Ac or pGlu pyroglutamic acid
  VARIANT 3: note = Aib, Ala, D-Ala or Gly
  VARIANT 13: note = Lys, Ile, or a Lys residue wherein the side chain of said Lys residue is conjugated to a lipophilic substituent
  VARIANT 16: note = Asp or Glu
  VARIANT 17: note = Ser, Glu, Lys or a Lys residue wherein the side chain of said Lys residue is conjugated to a lipophilic substituent
  VARIANT 18: note = Ile, Lys, Gln, Arg or a Lys residue wherein the side chain of said Lys residue is conjugated to a lipophilic substituent
  VARIANT 19: note = His, Arg or Ala
  VARIANT 20: note = Gln or Ala
  VARIANT 21: note = Gln, Lys, Ala, His or Arg
  VARIANT 22: note = Ala, Asp or Glu
  VARIANT 24: note = Ile or Val
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 43: note = Residue is absent or is a Lys residue wherein the side chain of said Lys residue is conjugated to a lipophilic substituent
  SITE 43: note = residue further connected to NH2 or OH
XYXEGTFISDYSXELXXXXXXXFXXXLXXXGKKNDWKHNITQX

SEQ ID NO: 108 - protein, 31 aa, synthetic construct
  VARIANT 1: note = H-, Ac or pGlu pyroglutamic acid
  VARIANT 3: note = Aib, Ala, D-Ala or Gly
  VARIANT 13: note = Lys, Ile, or a Lys residue wherein the side chain of said Lys residue is conjugated to a lipophilic substituent
  VARIANT 16: note = Asp or Glu
  VARIANT 17: note = Ser, Glu, Lys or a Lys residue wherein the side chain of said Lys residue is conjugated to a lipophilic substituent
  VARIANT 18: note = Ile, Lys, Gln, Arg or a Lys residue wherein the side chain of said Lys residue is conjugated to a lipophilic substituent
  VARIANT 19: note = His, Arg or Ala
  VARIANT 20: note = Gln or Ala
  VARIANT 21: note = Gln, Lys, Ala, His or Arg
  VARIANT 22: note = Ala, Asp or Glu
  VARIANT 24: note = Ile or Val
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 31: note = Residue is absent or is a Lys residue wherein the side chain of said Lys residue is conjugated to a lipophilic substituent
  SITE 31: note = residue further connected to NH2 or OH
XYXEGTFISDYSXELXXXXXXXFXXXLXXXX

SEQ ID NO: 109 - protein, 41 aa, synthetic construct
  VARIANT 1: note = H-, Ac or pGlu pyroglutamic acid
  SITE 3: note = Aib
  VARIANT 16: note = Asp or Glu
  VARIANT 17: note = Lys or a residue independently selected from Lys, Arg, Orn and Cys and wherein the side chain of said residue is conjugated to a lipophilic substituent
  VARIANT 18: note = Ile or a residue independently selected from Lys, Arg, Orn and Cys and wherein the side chain of said residue is conjugated to a lipophilic substituent
  VARIANT 19: note = His or Ala
  VARIANT 20: note = Gln or Ala
  VARIANT 21: note = Gln, Lys or Arg
  VARIANT 22: note = Ala, Asp or Glu
  VARIANT 25: note = Asn or Glu
  VARIANT 26: note = Tyr or Trp
  VARIANT 30: note = Gln or is absent
  VARIANT 41: note = Residue is absent or is a residue independently selected from Lys, Arg, Orn and Cys and wherein the side chain of said residue is conjugated to a lipophilic substituent
  SITE 41: note = residue further connected to NH2 or OH
XYXEGTFISDYSIELXXXXXXXFVXXLLAXGPSSGAPPPSX

SEQ ID NO: 110 - protein, 40 aa, synthetic construct
  VARIANT 1: note = H-, Ac or pGlu pyroglutamic acid
  SITE 3: note = Aib
  VARIANT 16: note = Asp or Glu
  VARIANT 17: note = Lys or a residue independently selected from Lys, Arg, Orn and Cys and wherein the side chain of said residue is conjugated to a lipophilic substituent
  VARIANT 18: note = Ile or a residue independently selected from Lys, Arg, Orn and Cys and wherein the side chain of said residue is conjugated to a lipophilic substituent
  VARIANT 19: note = His or Ala
  VARIANT 20: note = Gln or Ala
  VARIANT 21: note = Gln, Lys or Arg
  VARIANT 22: note = Ala, Asp or Glu
  VARIANT 25: note = Asn or Glu
  VARIANT 26: note = Tyr or Trp
  VARIANT 30: note = Gln or is absent
  VARIANT 40: note = Residue is absent or is a residue independently selected from Lys, Arg, Orn and Cys and wherein the side chain of said residue is conjugated to a lipophilic substituent
  SITE 40: note = residue further connected to NH2 or OH
XYXEGTFISDYSIELXXXXXXXFVXXLLAXGPSSGAPPSX

SEQ ID NO: 111 - protein, 40 aa, synthetic construct
  VARIANT 1: note = H-, Ac or pGlu pyroglutamic acid
  SITE 3: note = Aib
  VARIANT 16: note = Asp or Glu
  VARIANT 17: note = Lys or a residue independently selected from Lys, Arg, Orn and Cys and wherein the side chain of said residue is conjugated to a lipophilic substituent
  VARIANT 18: note = Ile or a residue independently selected from Lys, Arg, Orn and Cys and wherein the side chain of said residue is conjugated to a lipophilic substituent
  VARIANT 19: note = His or Ala
  VARIANT 20: note = Gln or Ala
  VARIANT 21: note = Gln, Lys or Arg
  VARIANT 22: note = Ala, Asp or Glu
  VARIANT 25: note = Asn or Glu
  VARIANT 26: note = Tyr or Trp
  VARIANT 30: note = Gln or is absent
  VARIANT 40: note = Residue is absent or is a residue independently selected from Lys, Arg, Orn and Cys and wherein the side chain of said residue is conjugated to a lipophilic substituent
  SITE 40: note = residue further connected to NH2 or OH
XYXEGTFISDYSIELXXXXXXXFVXXLLAXPSSGAPPPSX

SEQ ID NO: 112 - protein, 39 aa, synthetic construct
  VARIANT 1: note = H-, Ac or pGlu pyroglutamic acid
  SITE 3: note = Aib
  VARIANT 16: note = Asp or Glu
  VARIANT 17: note = Lys or a residue independently selected from Lys, Arg, Orn and Cys and wherein the side chain of said residue is conjugated to a lipophilic substituent
  VARIANT 18: note = Ile or a residue independently selected from Lys, Arg, Orn and Cys and wherein the side chain of said residue is conjugated to a lipophilic substituent
  VARIANT 19: note = His or Ala
  VARIANT 20: note = Gln or Ala
  VARIANT 21: note = Gln, Lys or Arg
  VARIANT 22: note = Ala, Asp or Glu
  VARIANT 25: note = Asn or Glu
  VARIANT 26: note = Tyr or Trp
  VARIANT 30: note = Gln or is absent
  VARIANT 39: note = Residue is absent or is a residue independently selected from Lys, Arg, Orn and Cys and wherein the side chain of said residue is conjugated to a lipophilic substituent
  SITE 39: note = residue further connected to NH2 or OH
XYXEGTFISDYSIELXXXXXXXFVXXLLAXPSSGAPPSX

SEQ ID NO: 113 - protein, 43 aa, synthetic construct
  VARIANT 1: note = H-, Ac or pGlu pyroglutamic acid
  SITE 3: note = Aib
  VARIANT 16: note = Asp or Glu
  VARIANT 17: note = Lys or a residue independently selected from Lys, Arg, Orn and Cys and wherein the side chain of said residue is conjugated to a lipophilic substituent
  VARIANT 18: note = Ile or a residue independently selected from Lys, Arg, Orn and Cys and wherein the side chain of said residue is conjugated to a lipophilic substituent
  VARIANT 19: note = His or Ala
  VARIANT 20: note = Gln or Ala
  VARIANT 21: note = Gln, Lys or Arg
  VARIANT 22: note = Ala, Asp or Glu
  VARIANT 25: note = Asn or Glu
  VARIANT 26: note = Tyr or Trp
  VARIANT 30: note = Gln or is absent
  VARIANT 43: note = Residue is absent or is a residue independently selected from Lys, Arg, Orn and Cys and wherein the side chain of said residue is conjugated to a lipophilic substituent
  SITE 43: note = residue further connected to NH2 or OH
XYXEGTFISDYSIELXXXXXXXFVXXLLAXGKKNDWKHNITQX

SEQ ID NO: 114 - protein, 31 aa, synthetic construct
  VARIANT 1: note = H-, Ac or pGlu pyroglutamic acid
  SITE 3: note = Aib
  VARIANT 16: note = Asp or Glu
  VARIANT 17: note = Lys or a residue independently selected from Lys, Arg, Orn and Cys and wherein the side chain of said residue is conjugated to a lipophilic substituent
  VARIANT 18: note = Ile or a residue independently selected from Lys, Arg, Orn and Cys and wherein the side chain of said residue is conjugated to a lipophilic substituent
  VARIANT 19: note = His or Ala
  VARIANT 20: note = Gln or Ala
  VARIANT 21: note = Gln, Lys or Arg
  VARIANT 22: note = Ala, Asp or Glu
  VARIANT 25: note = Asn or Glu
  VARIANT 26: note = Tyr or Trp
  VARIANT 30: note = Gln or is absent
  VARIANT 31: note = Residue is absent or is a residue independently selected from Lys, Arg, Orn and Cys and wherein the side chain of said residue is conjugated to a lipophilic substituent
  SITE 31: note = residue further connected to NH2 or OH
XYXEGTFISDYSIELXXXXXXXFVXXLLAXX

SEQ ID NO: 115 - protein, 33 aa, synthetic construct
  VARIANT 1: note = H-, Ac or pGlu pyroglutamic acid (pGlu; (S)-(-)-2-pyrrolidone-5-carboxylic acid), C1-4 alkyl, acetyl, formyl, benzoyl and trifluoroacetyl
  VARIANT 3: note = Aib, Ala, D-Ala, Gly, Ser, N-Me-Ser, Ac3c, Ac4c or Ac5c
  VARIANT 11: note = Tyr, Leu or Ser
  VARIANT 12: note = Ser or Leu
  VARIANT 13: note = Lys, Ile or a residue independently selected from Lys, Arg, Orn and Cys and wherein the side chain of said residue is conjugated to a lipophilic substituent
  VARIANT 16: note = Asp or Glu
  VARIANT 17: note = Ser, Glu, Lys, or a residue independently selected from Lys, Arg, Orn and Cys and wherein the side chain of said residue is conjugated to a lipophilic substituent
  VARIANT 18: note = Ile, Lys, Gln, Arg, or a residue independently selected from Lys, Arg, Orn and Cys and wherein the side chain of said residue is conjugated to a lipophilic substituent
  VARIANT 19: note = His, Arg or Ala
  VARIANT 20: note = Gln, Lys, Ala or Glu
  VARIANT 21: note = Gln, Lys, Ala, His or Arg
  VARIANT 22: note = Ala, Leu, Asp or Glu
  VARIANT 24: note = Val or Ile
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 33: note = Residue is absent or is a residue independently selected from Lys, Arg, Orn and Cys and wherein the side chain of said residue is conjugated to a lipophilic substituent
  SITE 33: note = residue further connected to NH2 or OH
XYXEGTFISDXXXELXXXXXXXFXXXLXXXKGX

SEQ ID NO: 116 - protein, 33 aa, synthetic construct
  VARIANT 1: note = H-, Ac or pGlu pyroglutamic acid
  VARIANT 3: note = Aib, Ala, D-Ala or Gly
  VARIANT 13: note = Lys, Ile, or a Lys residue wherein the side chain of said Lys residue is conjugated to a lipophilic substituent
  VARIANT 16: note = Asp or Glu
  VARIANT 17: note = Ser, Glu, Lys or a Lys residue wherein the side chain of said Lys residue is conjugated to a lipophilic substituent
  VARIANT 18: note = Ile, Lys, Gln, Arg or a Lys residue wherein the side chain of said Lys residue is conjugated to a lipophilic substituent
  VARIANT 19: note = His, Arg or Ala
  VARIANT 20: note = Gln or Ala
  VARIANT 21: note = Gln, Lys, Ala, His or Arg
  VARIANT 22: note = Ala, Asp or Glu
  VARIANT 24: note = Ile or Val
  VARIANT 25: note = Asn or Glu
  VARIANT 26: note = Tyr or Trp
  VARIANT 28: note = Leu, Glu, Ser, Lys or Val
  VARIANT 29: note = Ala, Ser or Arg
  VARIANT 30: note = Aib, Gly, Ala, Gln, Thr, Ser or Lys or is absent
  VARIANT 33: note = Residue is absent or is a Lys residue wherein the side chain of said Lys residue is conjugated to a lipophilic substituent
  SITE 33: note = residue further connected to NH2 or OH
XYXEGTFISDYSXELXXXXXXXFXXXLXXXKGX

SEQ ID NO: 117 - protein, 33 aa, synthetic construct
  VARIANT 1: note = H-, Ac or pGlu pyroglutamic acid
  SITE 3: note = Aib
  VARIANT 16: note = Asp or Glu
  VARIANT 17: note = Lys or a residue independently selected from Lys, Arg, Orn and Cys and wherein the side chain of said residue is conjugated to a lipophilic substituent
  VARIANT 18: note = Ile or a residue independently selected from Lys, Arg, Orn and Cys and wherein the side chain of said residue is conjugated to a lipophilic substituent
  VARIANT 19: note = His or Ala
  VARIANT 20: note = Gln or Ala
  VARIANT 21: note = Gln, Lys or Arg
  VARIANT 22: note = Ala, Asp or Glu
  VARIANT 25: note = Asn or Glu
  VARIANT 26: note = Tyr or Trp
  VARIANT 30: note = Gln or is absent
  VARIANT 33: note = Residue is absent or is a residue independently selected from Lys, Arg, Orn and Cys and wherein the side chain of said residue is conjugated to a lipophilic substituent
  SITE 33: note = residue further connected to NH2 or OH
XYXEGTFISDYSIELXXXXXXXFVXXLLAXKGX

SEQ ID NO: 118 - protein, 39 aa, synthetic construct
  SITE 3: note = Aib
  SITE 18: note = conjugated to ([19-carboxy-nonadecanoyl]-isoGlu-Peg3-Peg3)
HYXEGTFISDYSIELDKKAAQDFVNWLLAGPSSGAPPPS